SEQUENCE LISTING (16 sequences)

SEQ ID NO: 1 - DNA, 23 bp, synthetic construct
  misc_feature 1..23: note = Description of sequence: Description: Primer
  source 1..23: mol_type = other DNA
ccagagacaacgccaagaactca

SEQ ID NO: 2 - DNA, 22 bp, synthetic construct
  misc_feature 1..22: note = Description of sequence: Description: Primer
  source 1..22: mol_type = other DNA
ccccgctcccatggaaatatga

SEQ ID NO: 3 - DNA, 20 bp, synthetic construct
  misc_feature 1..20: note = Description of sequence: Description: Probe
  source 1..20: mol_type = other DNA
ccacagccacccccatccct

SEQ ID NO: 4 - DNA, 20 bp, synthetic construct
  misc_feature 1..20: note = Description of sequence: Description: Primer
  source 1..20: mol_type = other DNA
agcagccaaatccttcagtc

SEQ ID NO: 5 - DNA, 20 bp, synthetic construct
  misc_feature 1..20: note = Description of sequence: Description: Primer
  source 1..20: mol_type = other DNA
gcagcaaggtttttgtctgg

SEQ ID NO: 6 - DNA, 20 bp, synthetic construct
  misc_feature 1..20: note = Description of sequence: Description: Prober
  source 1..20: mol_type = other DNA
actcacagctgggggatgcc

SEQ ID NO: 7 - DNA, 15 bp, synthetic construct
  misc_feature 1..15: note = Description of sequence: Description: Primer
  source 1..15: mol_type = other DNA
aggcctggccgtgaa

SEQ ID NO: 8 - DNA, 16 bp, synthetic construct
  misc_feature 1..16: note = Description of sequence: Description: Primer
  source 1..16: mol_type = other DNA
cccagccagggcctta

SEQ ID NO: 9 - DNA, 23 bp, synthetic construct
  misc_feature 1..23: note = Description of sequence: Description: Probe
  source 1..23: mol_type = other DNA
acttgccagccccacagagcccc

SEQ ID NO: 10 - DNA, 15 bp, synthetic construct
  misc_feature 1..15: note = Description of sequence: Description: Primer
  source 1..15: mol_type = other DNA
agcagtgcagcccca

SEQ ID NO: 11 - DNA, 16 bp, synthetic construct
  misc_feature 1..16: note = Description of sequence: Description: Primer
  source 1..16: mol_type = other DNA
ctccaggccacagcac

SEQ ID NO: 12 - DNA, 23 bp, synthetic construct
  misc_feature 1..23: note = Description of sequence: Description: Probe
  source 1..23: mol_type = other DNA
ccaggcccgctgtgtccccagtt

SEQ ID NO: 13 - DNA, 168 bp, synthetic construct
  misc_feature 1..168: note = Description of sequence: Telomeric inversion junction sequence
  source 1..168: mol_type = other DNA
  gene 1..88: note = TRAJ
  gene 89..168: note = IGH
gccatttgtgcaggaggatcccagctcacttacttggtatgaccaccacttggttccccttcccaaaagcgagtctgttgttcccatatcgcacagtaataaacagccgtgtcctcggctctcaggctgttcatttgcagatacagtgagttcttggcgttgtctctg

SEQ ID NO: 14 - DNA, 1000 bp, synthetic construct
  misc_feature 1..1000: note = Description of sequence: Telomeric Inversion construct (gBlock) sequence used as POS CTRL
  source 1..1000: mol_type = other DNA
  gene 1..500: note = TRAJ
  gene 501..1000: note = IGH
ctgtgtggctccaccaaactcagggtgggagtaaagggctggggcccctccactgctgtgcactgaccatcagcacggtcttggacagatgctagaccctggcatcaggtccctctgcaacatgagcaagatgtccccacggtcccctccagctctcacttttgtggtcctttgttcaatctgaaatccagcctcagggaagatcaggggcagcttacatgagaatgggagtgtggtcagtggagatgcagagaaggggagagtgaaagaaaccagggacaggcaaagatttgggacagtggccgcctaaggttcttcgatatagcactgatgaatggcatgtccttcccccgctcccatggaaatatgataagaggctctccagcacagggtagggatgggggtggctgtggccatttgtgcaggaggatcccagctcacttacttggtatgaccaccacttggttccccttcccaaaagcgagtctgttgttcccatatcgcacagtaataaacagccgtgtcctcggctctcaggctgttcatttgcagatacagtgagttcttggcgttgtctctggagatggtgaatcggcccttcacagagtctgcgtagtatatggtactactactactaatggatgagacccactccagccccttccctggagcctggcggacccagttcatgtagtagtcactgaaggtgaatccagaggctgcacaggagagtctcagggaccccccaggctttaccaagcctcccccagactccaccagctgcacctcacactggacacctgcaaacaaagagacaacctggtcagaaacttccacacaaatccactgtttatctcactcttatccactcacactcaatttttctatttctccatgaattaccttttaaaatagccacaagaaaaagccagctcagccatgactccttggtggtcctctgtgttcagtcctgatcaccaaatgaaaacacctgaaaatc

SEQ ID NO: 15 - DNA, 210 bp, synthetic construct
  misc_feature 1..210: note = Description of sequence: RAG recombination signal sequence
  source 1..210: mol_type = other DNA
  variation 8..30: note = may be absent
  variation 38..60: note = may be absent
  variation 68..90: note = may be absent
  variation 98..120: note = may be absent
  variation 128..150: note = may be absent
  variation 158..180: note = may be absent
  variation 188..210: note = may be absent
cacagtgnnnnnnnnnnnnnnnnnnnnnnncacagtgnnnnnnnnnnnnnnnnnnnnnnncacagtgnnnnnnnnnnnnnnnnnnnnnnncacagtgnnnnnnnnnnnnnnnnnnnnnnncacagtgnnnnnnnnnnnnnnnnnnnnnnncacagtgnnnnnnnnnnnnnnnnnnnnnnncacagtgnnnnnnnnnnnnnnnnnnnnnnn

SEQ ID NO: 16 - DNA, 288 bp, synthetic construct
  misc_feature 1..288: note = Description of sequence: RAG recombination signal sequence
  source 1..288: mol_type = other DNA
  variation 10..32: note = may be absent
  variation 42..64: note = may be absent
  variation 74..96: note = may be absent
  variation 106..128: note = may be absent
  variation 138..160: note = may be absent
  variation 170..192: note = may be absent
  variation 200..224: note = may be absent
  variation 234..256: note = may be absent
  variation 266..288: note = may be absent
acaaaaaccnnnnnnnnnnnnnnnnnnnnnnnacaaaaaccnnnnnnnnnnnnnnnnnnnnnnnacaaaaaccnnnnnnnnnnnnnnnnnnnnnnnacaaaaaccnnnnnnnnnnnnnnnnnnnnnnnacaaaaaccnnnnnnnnnnnnnnnnnnnnnnnacaaaaaccnnnnnnnnnnnnnnnnnnnnnnnacaaaaaccnnnnnnnnnnnnnnnnnnnnnnnacaaaaaccnnnnnnnnnnnnnnnnnnnnnnnacaaaaaccnnnnnnnnnnnnnnnnnnnnnnn

(19) United States
(12) Patent Application Publication
(10) Pub. No.: US 2025/0207199 A1
(43) Pub. Date: Jun. 26, 2025

(54) METHODS FOR DETECTING GENOMIC ABNORMALITIES IN CELLS

(71) Applicant: ALLOGENE THERAPEUTICS, INC., South San Francisco, CA (US)

(72) Inventors: Barbra Johnson SASU, San Francisco, CA (US); Thomas Charles PERTEL, Millbrae, CA (US); Suhasni GOPALAKRISHNAN, San Mateo, CA (US)

(73) Assignee: ALLOGENE THERAPEUTICS, INC., South San Francisco, CA (US)

(21) Appl. No.: 18/970,541

(22) Filed: Dec. 5, 2024

Related U.S. Application Data
(63) Continuation of application No. PCT/US2023/068170, filed on Jun. 9, 2023.
(60) Provisional application No. 63/386,436, filed on Dec. 7, 2022.
(60) Provisional application No. 63/366,096, filed on Jun. 9, 2022.

(51) Int. Cl. C12Q 1/6883; C12Q 1/6851

(52) U.S. Cl. CPC C12Q 1/6883; C12Q 1/6851

(57) ABSTRACT

The instant disclosure relates to methods for detecting genome abnormalities or variants, such as chromosomal abnormalities or structural variations in cell populations, including engineered cell populations.

### CROSS REFERENCE TO RELATED APPLICATIONS

The present application is a continuation application of PCT/US2023/068170 filed Jun. 9, 2023, which claims the benefit of priority to U.S. Provisional Application No. 63/366,096, filed on Jun. 9, 2022; and U.S. Provisional Application No. 63/386,436, filed on Dec. 7, 2022, the contents of which are hereby incorporated by reference in their entireties.

### REFERENCE TO SEQUENCE LISTING

This application contains a Sequence Listing which has been submitted electronically in XML format and is hereby incorporated by reference in its entirety. Said XML copy, created on Jun. 2, 2023, is named AT-051-03WO_ST26.xml and is 30,695 bytes in size.

### FIELD OF THE INVENTION

The instant disclosure relates to methods for detecting genome abnormalities or variants, such as chromosomal abnormalities, e.g., chromosomal structural variations, in populations of cells. The methods may be used to for detection of genome abnormalities in engineered cells, such as CAR T cells, and cells obtained from patients who have been administered engineered cell drug products, such as CAR T cell drug products.

### BACKGROUND OF THE INVENTION

Assessing cell viability and health during the cell therapy manufacturing process is critical and challenging. Monitoring the state of cells at various stages of the drug product manufacturing process is important for maintaining overall cell health and functionality. Some cell-based therapeutic products are generated using various modification techniques (e.g., gene editing techniques) where identification and/or detection of genome abnormalities or variants (e.g., from off-target effects or not from off-target effects) would be advantageous in assessing viability and health. The identification and/or detection of genome abnormalities in cells may be appropriate at different stages of the cell therapy manufacturing process such as the donor cell stage involving cells from a healthy donor or cells from an autologous donor, i.e., a subject in need of cell therapy, prior to any modification. The modified or engineered cell stage, such as after a donor cell has been modified or engineered to (i) express one or more exogenous molecules, such as a chimeric antigen receptor and/or (ii) eliminate or reduce expression of one or more endogenous molecules, is also of interest. In addition, the identification and/or detection of genome abnormalities or variants in donor cells and/or engineered cells after in vitro or in vivo expansion is of interest.

### SUMMARY OF THE INVENTION

The instant disclosure provides compositions, kits, processes, and systems that allow for the monitoring of cell state to ensure cell health and viability is maintained during the drug product manufacturing process. Such monitoring is especially advantageous in the context of manufacturing engineered cells, such as engineered immune cells, that can be produced in large batches in a drug product manufacturing process to ensure overall cell health and viability and uniformity of cell dosage content. Provided herein are reagents and processes for assessing the viability and health of cells for use in cell-based therapies that may help improve the efficiency of manufacturing and drug product processes.

In one aspect, the present disclosure provides methods for detecting a genome abnormality in an engineered cell population. In one embodiment, the method comprises providing genomic deoxyribonucleic acid (gDNA) molecules from an engineered cell population, which is suspected of having a genome abnormality or variant (such as a chromosome inversion) and has an edited genomic region. In another embodiment, the engineered cell population (i) is suspected of comprising a chromosome 14 inversion characterized by a centromeric inversion site at the T cell receptor alpha/delta locus (TCR A/D) and a telomeric inversion site at the immunoglobulin heavy chain (IGH) variable region and (ii) comprises an edited genomic region. In other embodiments, the method further comprises subjecting a gDNA molecule to conditions sufficient to generate a nucleic acid extension product or products, wherein the nucleic acid extension product(s) comprises a sequence corresponding to the chromosome 14 inversion or a complement thereof. The nucleic acid extension product(s) may be detected as an indicator of the presence of the chromosome 14 inversion in the engineered cell population.

In one embodiment, the centromeric inversion site of the chromosome 14 inversion is (i) present at a first genomic region and/or (ii) at the T cell receptor joining gene TRAJ7. In other embodiments, the TCR A/D locus is at 14q11. In another embodiment, the TCR A/D locus is at 14q11.2. In one other embodiment, the telomeric inversion site of the chromosome 14 inversion is (i) present at a second genomic region and/or (ii) at the IGHV3-69-1 pseudogene. In one embodiment, the IGHV3-69-1 pseudogene is within the IGH variable region. In one other embodiment, the IGH variable region is at 14q32. In another embodiment, the chromosome 14 inversion is (i) located between TRAJ7 and IGHV3-69-1 and/or (ii) a 14q11 inversion. In another embodiment, the edited genomic region of the engineered cell population is present at a third genomic region. In one additional embodiment, the third genomic region is different from the first genomic region and/or the second genomic region. The third genomic region, i.e., the edited genomic region, comprises a T Cell Receptor Alpha Constant (TRAC) gene and/or a CD52 gene. In other embodiments, the first genomic region and/or the second genomic region are separated from the third genomic region by at least about 5 kilobases (kb), about 6 kb, about 7 kb, about 8 kb, about 9 kb, about 10 kb, about 11 kb, about 12 kb, about 13 kb, about 14 kb, or about 15 kb.

In some embodiments, the chromosome 14 inversion occurred independent of a gene-editing process. The gene-editing process can be an in vitro or an in vivo gene-editing process. In another embodiment, the chromosome 14 inversion occurred spontaneously.

In other embodiments, the methods for detecting a genome abnormality in an engineered cell population further comprises subjecting the gDNA to conditions sufficient to generate a control nucleic acid extension product. The method can further comprise detecting the control nucleic acid extension product as an indicator that the conditions were sufficient to generate the control nucleic acid extension product. In addition, the method can further comprise providing additional gDNA molecules from an additional engineered cell population. The additional engineered cell population may be characterized by (i) lacking or not comprising the chromosome 14 inversion and (ii) comprising the edited genomic region. In one embodiment, an additional gDNA molecule from the additional engineered cell does not comprise the chromosome 14 inversion. The method can further comprise subjecting the additional gDNA molecule to conditions sufficient to generate (i) an additional nucleic acid extension product comprising a sequence corresponding to the chromosome 14 inversion or a complement thereof and (ii) an additional control nucleic acid extension product. In another embodiment, the method further comprises detecting the additional control nucleic acid extension product without detecting the additional nucleic acid extension product as (i) an indicator of the absence of the chromosome 14 inversion in the additional engineered cell population and/or (ii) an indicator that the conditions were sufficient to generate the control nucleic acid extension product. In other embodiment, the control nucleic acid extension product comprises a sequence corresponding to an endogenous gene sequence. The endogenous gene sequence may be a human ribonuclease P protein subunit p30 (RPP30) gene sequence.

In another embodiment, the method further comprises extracting gDNA from the population of cells prior to step a). The extracted gDNA may be fragmented to provide the gDNA molecules.

In other embodiments, the engineered cell population is an engineered immune cell population. In one embodiment, the engineered immune cell population comprises a chimeric antigen receptor (CAR) nucleic acid sequence. In another embodiment, the CAR nucleic acid sequence expresses a CAR that binds to BCMA, EGFRVIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, FLT3, CD70, DLL3, CD52 or CD34.

In another aspect, the present disclosure provides methods for detecting a genome abnormality in an engineered cell population using partition-based approaches. In one embodiment, the partition-based method comprises providing gDNA molecules from an engineered cell population, which has or is suspected of having a genome abnormality or variant (such as a chromosome inversion) and has an edited genome region. In another embodiment, the engineered cell population (to be analyzed using a partition-based method) (i) is suspected of comprising a chromosome 14 inversion characterized by a centromeric inversion site at the T cell receptor alpha/delta locus (TCR A/D) and a telomeric inversion site at the immunoglobulin heavy chain (IGH) variable region and (ii) comprises an edited genomic region. In one embodiment, the centromeric inversion site of the chromosome 14 inversion is (i) present at a first genomic region and/or (ii) at the T cell receptor joining gene TRAJ7. In other embodiments, the TCR A/D locus is at 14q11. In another embodiment, the TCR A/D locus is at 14q11.2. In one other embodiment, the telomeric inversion site of the chromosome 14 inversion is (i) present at a second genomic region and/or (ii) at the IGHV3-69-1 pseudogene. In one embodiment, the IGHV3-69-1 pseudogene is within the IGH variable region. In one other embodiment, the IGH variable region is at 14q32. In another embodiment, the chromosome 14 inversion is (i) located between TRAJ7 and IGHV3-69-1 and/or (ii) a 14q11 inversion. In another embodiment, the edited genomic region of the engineered cell population is present at a third genomic region. In one additional embodiment, the third genomic region is different from the first genomic region and/or the second genomic region. The third genomic region, i.e., the edited genomic region, comprises a T Cell Receptor Alpha Constant (TRAC) gene and/or a CD52 gene. In other embodiments, the first genomic region and/or the second genomic region are separated from the third genomic region by at least about 5 kilobases (kb), about 6 kb, about 7 kb, about 8 kb, about 9 kb, about 10 kb, about 11 kb, about 12 kb, about 13 kb, about 14 kb, or about 15 kb. In some embodiments, the chromosome 14 inversion occurred independent of a gene-editing process, which can be an in vitro or an in vivo gene-editing process. In one embodiment, the chromosome 14 inversion occurred spontaneously.

In other embodiments, the method further comprises generating a plurality of partitions. On or more partitions may contain a gDNA molecule and primer molecules that are complementary to a genomic region corresponding to the chromosome 14 inversion. In some embodiments, the primer molecules comprise a pair of primer molecules. In another embodiment, the method further comprises the step of generating, in the partition, amplicons from the gDNA molecule with the primer molecules. In some embodiments, the amplicons comprise sequences corresponding to the chromosome 14 inversion. The method may further include detecting the amplicons in the partition. In another embodiment, the method further comprises counting the amplicons in the partition. In one embodiment, the counting of the amplicons quantitates a number of chromosome 14 inversions in the genomes of the engineered cell population. In a further embodiment, the method further comprises counting a number of partitions which comprise amplicons, thereby quantitating a number of chromosome 14 inversions. In other embodiments, a subset of the plurality of partitions comprises no more than one gDNA molecule per partition or substantially all of the plurality of partitions comprise no more than one gDNA molecule.

In other embodiments, an additional partition of the plurality of partitions comprises an additional gDNA molecule and additional primer molecules that are complementary to the genomic region corresponding to the chromosome 14 inversion. In one embodiment, the additional primer molecules comprise an additional pair of primer molecules. The method can further comprise subjecting the additional gDNA molecule to conditions sufficient to generate, in the partition, (i) additional amplicons comprising a sequence corresponding to the chromosome 14 inversion or a complement thereof and (ii) a control nucleic acid extension product. In addition, the method can comprise generating and/or detecting the additional amplicons in the additional partition.

In other embodiments, the method further comprises generating and/or detecting the control nucleic acid extension product without generating and/or detecting the additional amplicons in the additional partition, wherein detecting the control nucleic acid extension product without detecting the additional amplicons in the additional partition is (i) an indicator of the absence of the chromosome 14 inversion in the additional gDNA molecule and/or (ii) an indicator that the conditions were sufficient to generate the control nucleic acid extension product. In other embodiment, the control nucleic acid extension product comprises a sequence corresponding to an endogenous gene sequence. The endogenous gene sequence may be a human ribonuclease protein subunit p30 (RPP30) gene sequence.

In some embodiments, the method further comprises extracting gDNA from the engineered cell prior to the step of providing gDNA molecules. The extracting step may further comprise fragmenting the extracted gDNA to provide the gDNA molecules.

In other embodiments, the engineered cell population being analyzed using a partition-based method is an engineered immune cell population. In one embodiment, the engineered immune cell population comprises a chimeric antigen receptor (CAR) nucleic acid sequence. In another embodiment, the CAR nucleic acid sequence expresses a CAR that binds to BCMA, EGFRVIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, FLT3, CD70, DLL3, CD52 or CD34.

In one other aspect, the present disclosure provides in vitro methods for detecting a genome abnormality in a patient cell population. In one embodiment, the method comprises providing genomic deoxyribonucleic acid (gDNA) molecules from a patient cell population. The patient cell population may comprises an immune cell which comprises or is suspected of comprising a genome abnormality or variant (such as a chromosome inversion) and comprises an edited genomic region. In one other embodiment, the immune cell (i) comprises or is suspected of comprising a chromosome 14 inversion characterized by a centromeric inversion site at the T cell receptor alpha/delta locus (TCR A/D) and a telomeric inversion site at the immunoglobulin heavy chain (IGH) variable region and (ii) comprises an edited genomic region. In one embodiment, the centromeric inversion site of the chromosome 14 inversion is (i) present at a first genomic region and/or (ii) at the T cell receptor joining gene TRAJ7. In other embodiments, the TCR A/D locus is at 14q11. In another embodiment, the TCR A/D locus is at 14q11.2. In one other embodiment, the telomeric inversion site of the chromosome 14 inversion is (i) present at a second genomic region and/or (ii) at the IGHV3-69-1 pseudogene. In one embodiment, the IGHV3-69-1 pseudogene is within the IGH variable region. In one other embodiment, the IGH variable region is at 14q32. In another embodiment, the chromosome 14 inversion is (i) located between TRAJ7 and IGHV3-69-1 and/or (ii) a 14q11 inversion. As described in Example 1, next-generation sequencing (NGS) was used to examine the precise breakpoints of an inversion in a patient. Sequencing confirmed the presence of the chromosome 14 inversion and identified the inversion breakpoint sites (see FIG. 2). The centromeric (14q11.2) inversion site occurred within the TCR A/D locus at the start of the T cell receptor joining gene TRAJ7 (chr14: 22537626; hg38 reference genome build). The telomeric (14q32) inversion site occurred at the IGHV3-69-1 pseudogene (chr14: 106728167; hg38 reference genome build) within the IGH variable region.

In another embodiment, the edited genomic region of the immune cell population is present at a third genomic region. In one additional embodiment, the third genomic region is different from the first genomic region and/or the second genomic region. The third genomic region, i.e., the edited genomic region, comprises a T Cell Receptor Alpha Constant (TRAC) gene and/or a CD52 gene. In other embodiments, the first genomic region and/or the second genomic region are separated from the third genomic region by at least about 5 kilobases (kb), about 6 kb, about 7 kb, about 8 kb, about 9 kb, about 10 kb, about 11 kb, about 12 kb, about 13 kb, about 14 kb, or about 15 kb.

In one additional embodiment, the patient cell population originated from a patient treated with a population of engineered immune cells. In another embodiment, the chromosome 14 inversion was present or absent in the population of engineered immune cells. In other embodiments, the chromosome 14 inversion occurred in the immune cell following treatment with the population of engineered immune cells. In some embodiments, the chromosome 14 inversion occurred independent of a gene-editing process, which can be an in vitro or an in vivo gene-editing process. In one embodiment, the chromosome 14 inversion occurred spontaneously.

In another embodiment, the method further comprises subjecting a gDNA molecule to conditions sufficient to generate a nucleic acid extension product. In another embodiment, the nucleic acid extension product comprises a sequence corresponding to the chromosome 14 inversion or a complement thereof. The method may further comprise detecting the nucleic acid extension product as an indicator of the presence of the chromosome 14 inversion in the immune cell.

In other embodiments, the method further comprises subjecting the gDNA to conditions sufficient to generate a control nucleic acid extension product. The method can further include detecting the control nucleic acid extension product as an indicator that the conditions were sufficient to generate the control nucleic acid extension product. In other embodiments, the control nucleic acid extension product comprises a sequence corresponding to an endogenous gene sequence, such as a human ribonuclease P protein subunit p30 (RPP30) gene sequence.

In other embodiments, the immune cell from the patient cell population being analyzed using an in vitro method is an immune cell comprising a chimeric antigen receptor (CAR) nucleic acid sequence. In another embodiment, the CAR nucleic acid sequence expresses a CAR that binds to BCMA, EGFRVIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, FLT3, CD70, DLL3, CD52 or CD34. The immune cell from the patient cell population may be an engineered immune cell previously administered to the patient or an immune cell that is part of a clonal population of the previously administered engineered immune cells, e.g., a clonal population of CAR T cells following in vivo expansion.

In one additional aspect, the present disclosure provides a drug product release assay for an allogeneic immune cell drug product. In one embodiment, the release assay comprises a step of providing an allogeneic immune cell drug product comprising engineered immune cells that comprise edited genomic regions. The release assay can further comprise a step of extracting gDNA molecules from the engineered immune cells. In addition, the release assay can comprise a step of subjecting a gDNA molecule to conditions sufficient to generate a nucleic acid extension product. The nucleic acid extension comprises a sequence corresponding to a genome abnormality or variant (such as a chromosome inversion). In one embodiment, the nucleic acid extension comprises a sequence corresponding to a chromosome 14 inversion characterized by a centromeric inversion site at the T cell receptor alpha/delta locus (TCR A/D) and a telomeric inversion site at the immunoglobulin heavy chain (IGH) variable region, or a complement thereof. In another embodiment, the chromosome 14 inversion occurred independent of a gene-editing process, which can be an in vitro or an in vivo gene-editing process. In other embodiments, the chromosome inversion occurred spontaneously. In one embodiment, the centromeric inversion site of the chromosome 14 inversion is (i) present at a first genomic region and/or (ii) at the T cell receptor joining gene TRAJ7. In other embodiments, the TCR A/D locus is at 14q11. In another embodiment, the TCR A/D locus is at 14q11.2. In one other embodiment, the telomeric inversion site of the chromosome 14 inversion is (i) present at a second genomic region and/or (ii) at the IGHV3-69-1 pseudogene. In one embodiment, the IGHV3-69-1 pseudogene is within the IGH variable region. In one other embodiment, the IGH variable region is at 14q32. In another embodiment, the chromosome 14 inversion is (i) located between TRAJ7 and IGHV3-69-1 and/or (ii) a 14q11 inversion. In another embodiment, the edited genomic region of the immune cell population is present at a third genomic region. In one additional embodiment, the third genomic region is different from the first genomic region and/or the second genomic region. The third genomic region, i.e., the edited genomic region, comprises a T Cell Receptor Alpha Constant (TRAC) gene and/or a CD52 gene. In other embodiments, the first genomic region and/or the second genomic region are separated from the third genomic region by at least about 5 kilobases (kb), about 6 kb, about 7 kb, about 8 kb, about 9 kb, about 10 kb, about 11 kb, about 12 kb, about 13 kb, about 14 kb, or about 15 kb.

In a further embodiment, the release assay comprises detecting the nucleic acid extension product as an indicator of the presence of the chromosome 14 inversion in the engineered immune cells.

In another embodiment, the extracting step of the release assay comprises fragmenting the gDNA to provide the gDNA molecules.

In other embodiments, the release assay further comprises a step of subjecting the gDNA to conditions sufficient to generate a control nucleic acid extension product. The release assay can further comprise detecting the control nucleic acid extension product as an indicator that the conditions were sufficient to generate the control nucleic acid extension product. In other embodiments, the release assay further comprises a step of extracting additional gDNA molecules from additional engineered immune cells. The additional engineered immune cell (i) does not comprise the chromosome 14 inversion and (ii) comprises the edited genomic region. In addition, the release assay comprises a step of subjecting an additional gDNA molecule to conditions sufficient to generate (i) an additional nucleic acid extension product comprising a sequence corresponding to the chromosome 14 inversion or a complement thereof and (ii) an additional control nucleic acid extension product. The release assay can further include a step of detecting the additional control nucleic acid extension product without detecting the additional nucleic acid extension product as an indicator (i) of the absence of the chromosome 14 inversion in the additional engineered immune cells and/or (ii) that the conditions were sufficient to generate the control nucleic acid extension product. In other embodiments, the control nucleic acid extension product comprises a sequence corresponding to an endogenous gene sequence, such as a human ribonuclease P protein subunit p30 (RPP30) gene sequence.

In other embodiments, engineered immune cells from the drug product being analyzed using the release assay are engineered immune cells comprising a chimeric antigen receptor (CAR) nucleic acid sequence. In another embodiment, the CAR nucleic acid sequence expresses a CAR that binds to BCMA, EGFRVIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, FLT3, CD70, DLL3, CD52 or CD34.

In certain embodiments, the engineered immune cells are T cells, inflammatory T-lymphocytes, cytotoxic T-lymphocytes, regulatory T-lymphocytes, helper T-lymphocytes, effector T-lymphocytes, tumor infiltrating lymphocytes (TILs), NK cells, NK-T-cells, TCR-expressing cells, TCR knockout T cells, dendritic cells, macrophages, killer dendritic cells, mast cells, or B-cells. In certain embodiments, the engineered immune cells are CAR T cells. In certain embodiments, the engineered immune cells are allogeneic CAR T cells. In certain embodiments, the engineered immune cells are autologous CAR T cells. In some embodiments, the engineered immune cells are human cells.

In another aspect, the instant disclosure provides a drug product comprising engineered immune cells prepared by the drug product process disclosed herein.

In yet another aspect, the instant disclosure provides methods of treating a subject in need of a treatment comprising administering to the subject the drug product disclosed herein. In certain embodiments, the subject is a human. In some embodiments, the treatment step is subsequent to the outcome of the methods or release assay steps described herein.

The presence of genome abnormalities in an allogeneic immune cell drug product can raise safety concerns that may prevent administration of the drug product to a subject. In one additional aspect, the present disclosure provides methods for confirming the safety of an allogeneic immune cell drug product. In one embodiment, the method for confirming the safety of an allogeneic immune cell drug product comprises a step of providing an allogeneic immune cell drug product comprising engineered immune cells that comprise edited genomic regions. The method can further comprise a step of extracting gDNA molecules from the engineered immune cells. In addition, the method can comprise a step of subjecting a gDNA molecule to conditions sufficient to generate a nucleic acid extension product. The nucleic acid extension comprises a sequence corresponding to a genome abnormality or variant (such as a chromosome inversion). In one embodiment, the nucleic acid extension comprises a sequence corresponding to a chromosome 14 inversion characterized by a centromeric inversion site at the T cell receptor alpha/delta locus (TCR A/D) and a telomeric inversion site at the immunoglobulin heavy chain (IGH) variable region, or a complement thereof. In another embodiment, the chromosome 14 inversion occurred independent of a gene-editing process, which can be an in vitro or an in vivo gene-editing process. In other embodiments, the chromosome inversion occurred spontaneously. In one embodiment, the centromeric inversion site of the chromosome 14 inversion is (i) present at a first genomic region and/or (ii) at the T cell receptor joining gene TRAJ7. In other embodiments, the TCR A/D locus is at 14q11. In another embodiment, the TCR A/D locus is at 14q11.2. In one other embodiment, the telomeric inversion site of the chromosome 14 inversion is (i) present at a second genomic region and/or (ii) at the IGHV3-69-1 pseudogene. In one embodiment, the IGHV3-69-1 pseudogene is within the IGH variable region. In one other embodiment, the IGH variable region is at 14q32. In another embodiment, the chromosome 14 inversion is (i) located between TRAJ7 and IGHV3-69-1 and/or (ii) a 14q11 inversion. In another embodiment, the edited genomic region of the immune cell population is present at a third genomic region. In one additional embodiment, the third genomic region is different from the first genomic region and/or the second genomic region. The third genomic region, i.e., the edited genomic region, comprises a T Cell Receptor Alpha Constant (TRAC) gene and/or a CD52 gene. In other embodiments, the first genomic region and/or the second genomic region are separated from the third genomic region by at least about 5 kilobases (kb), about 6 kb, about 7 kb, about 8 kb, about 9 kb, about 10 kb, about 11 kb, about 12 kb, about 13 kb, about 14 kb, or about 15 kb.

In a further embodiment, the method comprises detecting the nucleic acid extension product as an indicator of the presence of the chromosome 14 inversion in the engineered immune cells, thereby confirming the safety of an allogeneic immune cell drug product.

In another embodiment, the extracting step of the method for confirming the safety of an allogeneic immune cell drug product comprises fragmenting the gDNA to provide the gDNA molecules.

In other embodiments, the method for confirming the safety of an allogeneic immune cell drug product further comprises a step of subjecting the gDNA to conditions sufficient to generate a control nucleic acid extension product. The method can further comprise detecting the control nucleic acid extension product as an indicator that the conditions were sufficient to generate the control nucleic acid extension product. In other embodiments, the method further comprises a step of extracting additional gDNA molecules from additional engineered immune cells. The additional engineered immune cell (i) does not comprise the chromosome 14 inversion and (ii) comprises the edited genomic region. In addition, the method comprises a step of subjecting an additional gDNA molecule to conditions sufficient to generate (i) an additional nucleic acid extension product comprising a sequence corresponding to the chromosome 14 inversion or a complement thereof and (ii) an additional control nucleic acid extension product. The method can further include a step of detecting the additional control nucleic acid extension product without detecting the additional nucleic acid extension product as an indicator (i) of the absence of the chromosome 14 inversion in the additional engineered immune cells and/or (ii) that the conditions were sufficient to generate the control nucleic acid extension product. In other embodiments, the control nucleic acid extension product comprises a sequence corresponding to an endogenous gene sequence, such as a human ribonuclease P protein subunit p30 (RPP30) gene sequence.

In other embodiments, engineered immune cells from the drug product being analyzed using the method are engineered immune cells comprising a chimeric antigen receptor (CAR) nucleic acid sequence. In another embodiment, the CAR nucleic acid sequence expresses a CAR that binds to BCMA, EGFRVIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, FLT3, CD70, DLL3, CD52 or CD34.

In certain embodiments, the engineered immune cells are T cells, inflammatory T-lymphocytes, cytotoxic T-lymphocytes, regulatory T-lymphocytes, helper T-lymphocytes, effector T-lymphocytes, tumor infiltrating lymphocytes (TILs), NK cells, NK-T-cells, TCR-expressing cells, TCR knockout T cells, dendritic cells, macrophages, killer dendritic cells, mast cells, or B-cells. In certain embodiments, the engineered immune cells are CAR T cells. In certain embodiments, the engineered immune cells are allogeneic CAR T cells. In certain embodiments, the engineered immune cells are autologous CAR T cells. In some embodiments, the engineered immune cells are human cells.

### BRIEF DESCRIPTION OF THE DRAWINGS

FIG. 1A shows longitudinal measurement of CAR T cell VCN, ALC and TCRβ clones in a patient. FIG. 1B demonstrates that the dominant clone from patient was not detected in the drug product. FIG. 1C depicts a break apart FISH probe configuration. FIG. 1D shows the % of a dominant clone.

FIG. 2 shows a map of chromosome 14 inversion detected and characterized from peripheral blood sample of the patient at Day 61 by short and long-read whole genome sequencing.

FIG. 3A-3B depicts the method of development of a telomeric inversion junction ddPCR assay. FIG. 3A shows raw data from the ddPCR from the Quantasoft software. FIG. 3B shows a linear double stranded DNA template (gBlock™) that was designed for both the centromeric and telomeric sites of inversion based on NGS sequence information (SEQ ID NOs: 13 and 14).

FIG. 4 shows longitudinal detection of the telomeric breakpoint junction by ddPCR in patient peripheral blood samples.

FIG. 5 depicts a chromosomal translocation.

FIG. 6 shows a screening assay workflow for donor cell populations and/or drug products lots comprising engineered cells.

### DETAILED DESCRIPTION

The practice of the instant disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R.I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995). Gene editing techniques using TALENs, CRISPR/Cas9, and megaTAL nucleases, for example, are within the skill of the art and explained fully in the literature, such as T. Gaj et al., Genome-Editing Technologies: Principles and Applications, Cold Spring Harb Perspect Biol 2016; 8:a023754 and citations therein.

### Definitions

As used herein, the terms “a” and “an” are used to mean one or more. For example, a reference to “a cell” or “an antibody” means “one or more cells” or “one or more antibodies.”

As used herein “autologous” means that cells, a cell line, or population of cells used for treating subjects that are obtained from said subject.

As used herein “allogeneic” means that cells or population of cells used for treating subjects that are not obtained from said subject, but instead from a donor.

As used herein, the term “endogenous” refers to any material from or produced inside an organism, cell, tissue or system.

As used herein, the term “exogenous” refers to any material introduced from or produced outside an organism, cell, tissue or system.

As used herein, “immune cell” refers to a cell of hematopoietic origin functionally involved in the initiation and/or execution of innate and/or adaptative immune response. Examples of immune cells include T cells, e.g., alpha/beta T cells and gamma/delta T cells, Regulatory T (Treg) cells, B cells, natural killer (NK) cells, natural killer T (NKT) cells, mast cells, and myeloid-derived phagocytes.

As used herein, an “edited genomic region” refers to a region of the genome within a cell of an organism that was the subject of genomic editing by a genomic editing reagent. Any manner of genomic editing may be suitable including, without limitation, point mutations, deletions, insertions, and replacements. For example, the genome may be subjected to genomic editing to insert or introduce a nucleic acid sequence encoding an exogenous molecule (e.g., an antigen binding domain) to allow the cell to express the exogenous molecule. In another example, the genome may be subjected to genomic editing to delete or disrupt the nucleic acid sequence of a gene expressing an endogenous molecule (e.g., a T cell receptor) such that expression of the endogenous molecule is reduced or eliminated in the cell.

As used herein, a “genomic editing reagent” refers to one or more reagents that can be used for editing of a region of the genome within a cell. In general, the one or more reagents include an enzyme and a targeting component. In one embodiment, the targeting component is (or is derived or isolated from) a transcription factor domain or a transcription activator-like (TAL) effector DNA binding domain. The targeting component may be a TAL effector DNA binding domain that has been coupled to (e.g., fused to) an enzyme, such as a nuclease. Alternatively, the targeting component may be a zinc-finger domain coupled to (e.g., fused to) a nuclease. In another embodiment, the targeting component can contain nucleic acids, e.g., guide RNAs, such as those utilized in CRISPR/Cas-type systems. Examples of nucleases include, without limitation, a type IIS restriction endonuclease (e.g., Fok I), a Cas nuclease (e.g., Cas9), or a derivative thereof.

As used herein, the term “nucleic acid product” generally refers to a nucleic acid product generated through one or more of hybridization, ligation, extension, transcription, reverse transcription, and amplification. A nucleic acid extension product is generated through the use of a polymerase (e.g., a DNA or an RNA polymerase), a template nucleic acid molecule (e.g., genomic DNA), nucleotides, and a primer molecule. A nucleic acid amplification product is generated in a manner similar to that for a nucleic acid extension product but involves the use of more than one primer. A nucleic acid hybridization product is generated via binding of a first nucleic acid molecule, e.g., a primer molecule or a probe molecule, to a second nucleic acid molecule, e.g., a template nucleic acid. The first nucleic acid molecule (such as a probe molecule) may optionally comprise a label or moiety (e.g., a detectable label or detectable moiety, or a reporter label or reporter moiety). A nucleic acid transcription product is generated through the use of a polymerase (e.g., a RNA polymerase), a DNA template, and a primer molecule. A reverse transcription product is generated through the use of a polymerase (e.g., a DNA polymerase), an RNA template, and a primer molecule. A nucleic acid ligation product is generated through the use of a ligase (e.g., a DNA or RNA ligase), a template molecule (e.g., DNA or RNA template), and two or more probe molecules. Some or all of a probe molecule is complementary to a region of the template. In one embodiment, two probes are complementary to two regions of a template that are adjacent to one another. In another embodiment, there is a gap between the two probes upon hybridization to the template, which can be filled with a polymerase. Nucleic acid product may be generated from (i) sample nucleic acid to provide sample nucleic acid products or (ii) control nucleic acid to provide control nucleic acid products, as further described herein. In general, sample nucleic acid products may comprise a target sequence while control nucleic acid products do not comprise the target sequence.

As used herein, the term nucleic acid “amplification” or a nucleic acid “amplification reaction” refers to any in vitro method or process for creating nucleic acid strands that are the same or complementary to at least a portion of a template nucleic acid molecule. Generally, nucleic acid amplification is used to generate multiple copies of a template nucleic acid molecule or a complement thereof through the use of one or more polymerases and/or transcriptases. Examples of in vitro nucleic acid amplification include, without limitation, Polymerase Chain Reaction (PCR), Reverse Transcriptase-PCR (RT-PCR), Replicase Mediated Amplification, and Ligase Chain Reaction (LCR), Transcription-Mediated Amplification (TMA), Nucleic Acid Sequence-Based Amplification (NASBA), as well as isothermal amplification reactions (e.g., single-primer isothermal amplification (SPIA)). The process of “amplifying” involves subjecting an aqueous reaction to conditions sufficient to allow for amplification of a target or template nucleic acid molecule in the presence of all necessary reaction components. Components of an amplification reaction include, without limitation, one or more primers, a nucleic acid template, a polymerase, and nucleotides. The term “amplifying” typically refers to an exponential increase in target nucleic acid but “amplifying” can also refer to linear increases in the numbers of a target sequence from a nucleic acid template, such as is obtained with cycle sequencing or linear amplification. The process of “amplification” or the step of “amplifying” produces an amplification product, or “amplicon.”

As used herein, the term “reaction mixture” refers to an aqueous solution comprising the different reagents that are used to generate a nucleic acid product. The reagents include, without limitation, enzymes, aqueous buffers, salts, primers, target or template nucleic acid, nucleoside triphosphates, stabilizers, and other additives to improve or optimize efficiency and/or specificity. The reaction mixture may be a ligation reaction mixture, an extension reaction mixture, a transcription reaction mixture, a reverse transcription reaction mixture, and an amplification reaction mixture.

“Oligonucleotide” as used herein refers to linear oligomers of natural or modified nucleosidic monomers linked by phosphodiester bonds or analogs thereof. Oligonucleotides include deoxyribonucleosides, ribonucleosides, anomeric forms thereof, peptide nucleic acids (PNAs), and the like, capable of specifically binding to a target nucleic acid. Usually monomers are linked by phosphodiester bonds or analogs thereof to form oligonucleotides ranging in size from a few monomeric units, e.g., 3-4, to several tens of monomeric units, e.g., 40-60. Whenever an oligonucleotide is represented by a sequence of letters, such as “ATGCCTG,” it will be understood that the nucleotides are in 5′-3′ order from left to right and that “A” denotes deoxyadenosine, “C” denotes deoxycytidine, “G” denotes deoxyguanosine, “T” denotes deoxythymidine, and “U” denotes the ribonucleoside, uridine, unless otherwise noted. Usually oligonucleotides comprise the four natural deoxynucleotides; however, they may also comprise ribonucleosides or non-natural nucleotide analogs. Where an enzyme has specific oligonucleotide or polynucleotide substrate requirements for activity, e.g., single stranded DNA, RNA/DNA duplex, or the like, then selection of appropriate composition for the oligonucleotide or polynucleotide substrates is well within the knowledge of one of ordinary skill.

As used herein, the term “polymerase chain reaction” or “PCR” refers to a method whereby a specific segment or sequence (e.g. a subsequence or a partial sequence) of a target double-stranded nucleic acid molecule (e.g., DNA or RNA), is amplified in a geometric progression. PCR is well known to those of skill in the art; see, e.g., U.S. Pat. Nos. 4,683,195 and 4,683,202, as well as PCR Protocols: A Guide to Methods and Applications, Innis et al., eds, 1990. Exemplary PCR reaction conditions typically comprise either two or three step cycles. Two step cycles have a denaturation step followed by a hybridization/elongation step. Three step cycles comprise a denaturation step followed by a hybridization step followed by a separate elongation step.

As used herein, a “primer molecule” or “primer” refers to a polynucleotide sequence that couples to (e.g., hybridizes to) a specific sequence on a template or target nucleic acid where it acts to prime (or initiate) nucleic acid synthesis. Primers can be of a variety of lengths and are often less than 50 nucleotides in length, for example 12-30 nucleotides, in length. The length and sequences of primers for use in nucleic acid synthesis reactions (e.g., PCR) can be designed based on principles known to those of skill in the art. Primers can comprise DNA and/or RNA, including a chimera of DNA and RNA portions. In some cases, primers can include one or more modified or non-natural nucleotide bases. In some cases, primers include a label or moiety (e.g., a detectable label or detectable moiety, or a reporter label or reporter moiety). Primers that initiate the amplification (e.g., PCR) of a target or template polynucleotide molecule are referred to as “amplification primers.” In non-amplification embodiments, a primer may be used to create a structure that is capable of being cleaved by a cleavage agent.

As used herein, the term “probe” refers to a molecule (e.g., a nucleic acid, polypeptide or protein, aptamer, etc.) that specifically binds to (or specifically interacts with) a target molecule, e.g., a target nucleic acid or polynucleotide, to allow for specific detection of the target molecule. Non-limiting examples of molecules that specifically bind to or interact with a target molecule, e.g., a target nucleic acid or polynucleotide, include nucleic acids (e.g., oligonucleotides), proteins (e.g., antibodies, transcription factors, zinc finger proteins, non-antibody protein scaffolds, etc.), and aptamers. Generally, a probe includes a label (e.g., a detectable label or detectable moiety, or a reporter label or reporter moiety). The probe can indicate the presence and/or amount of the target polynucleotide by either an increase or decrease in signal from the label. In some cases, the probes detect the target polynucleotide in an amplification reaction by being digested by the 5′ to 3′ exonuclease activity of a DNA dependent DNA polymerase. An “oligonucleotide probe” as used herein refers to a polynucleotide sequence capable of coupling to (e.g., hybridizing or annealing to) a target nucleic acid of interest and to allow for the specific detection of the target nucleic acid.

A “reporter moiety” or “reporter molecule” is a molecule that confers a detectable signal. The detectable phenotype can be colorimetric, fluorescent or luminescent, for example. A “quencher moiety” or “quencher molecule” is a molecule that is able to quench the detectable signal from the reporter moiety.

As used herein, a “control” or “control nucleic acid” is a nucleic acid having a control sequence from which a control nucleic acid product is generated and/or detected. The control nucleic acid product may be an external control generated in a control assay run alongside a sample assay. The control nucleic acid product may be an internal control generated in the same assay as a sample nucleic acid product. In general, control nucleic acid products (and/or their detection) are utilized to confirm that an assay is performing as expected. Droplet digital PCR (ddPCR) assays allow absolute quantification of nucleic acids and internal control nucleic acid can serve as an internal reference control for the amount of input genomic DNA material and/or enable assessment of the relative abundance of a target sequence (e.g., presence of an inversion) signal per genome (relative to the control sequence of the control nucleic acid). External control(s) for ddPCR assays include negative external controls (e.g., nucleic acid that does not have a target sequence) and positive external controls (e.g., nucleic acid that has the target sequence), which help ensure proper performance and reliability of the results of the sample assay.

As used herein, a “genomic abnormality”, a “genome abnormality” or a “chromosomal abnormality” refers to a variant, an anomaly, an aberration, or a mutation in the genome relative to a reference genome. Such abnormalities or variants can occur in the form of point mutation(s) or chromosomal structural variation(s). Structural variations include, without limitation, deletions, insertions, inversions, duplications (e.g., tandem or interspersed), and translocations. Such variations may be balance or unbalanced. Chromosomal structural variation(s) as used herein refers to chromosomal structural change(s) that occur and/or are detected in the genome of a cell as compared to the chromosomal structure of the wild type genome. In some embodiments, the cell is an engineered cell (e.g., an immune cell, such as a CAR T cell). In some embodiments, the cell is a CAR T cell. In other embodiments, the CAR T cell is an allogeneic CAR T cell derived from a donor cell. In an additional embodiment, the CAR T cell is an autologus CAR T cell derived from a subject who will ultimately receive the engineered cells.

In some embodiments, the chromosomal structural variation comprises a chromosomal inversion. In some embodiments, the chromosomal structural variation comprises a chromosomal translocation. An inversion or chromosomal inversion refers to a chromosomal rearrangement in which a segment of a chromosome is reversed end-to-end. An inversion can occur when a single chromosome undergoes double strand DNA breaks and rearranges within the same chromosome. A chromosomal translocation refers to a type of chromosomal abnormality in which a chromosome breaks and a portion of it reattaches to a different chromosome.

As used herein, the term “biological sample” or “sample” refers to a composition obtained from or derived from an individual subject or patient, wherein the composition includes a cell population. In one embodiment, the cell population is suspected of containing or contains one or more cells that have a variant or genome abnormality. The individual may be a donor (e.g., a healthy individual) or a patient (prior to, during, or after treatment with a therapeutic, e.g., a cellular therapeutic). Different types of biological samples include, without limitation, blood, peripheral blood mononuclear cells (PBMCs), bone marrow, and tissue biopsies. Biological samples may be obtained from an individual before treatment, during treatment or post-treatment. Samples may comprise any number of molecular constituents, such as nucleic acid and/or protein(s), from a cell population.

As used herein, the term “molecular constituent,” generally refers to a macromolecule contained within or from a cell or a cell population. The molecular constituent may comprise a nucleic acid, such as RNA or DNA. The DNA may be genomic deoxyribonucleic acid (gDNA) molecule, which can be fragmented (e.g., enzymatically fragmented). The RNA may be (i) coding or non-coding or (ii) messenger RNA (mRNA), ribosomal RNA (rRNA) transfer RNA (tRNA), 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), small rDNA-derived RNA (srRNA), a clustered regularly interspaced short palindromic (CRISPR) RNA molecule (crRNA), or a single guide RNA (sgRNA). The RNA may be a transcript or it may be double-stranded RNA or single-stranded RNA. The RNA may be circular RNA. The molecular constituent may comprise a protein, a peptide or a polypeptide.

### Analysis of Cell Populations

In one aspect, the present disclosure provides methods, assays, systems, compositions, and kits for the detection (and/or quantitation or counting) of one or more nucleic acid products generated from nucleic acid of a population of cells, such as engineered cells and/or cells obtained from a patient or subject. In general, nucleic acid products are generated from engineered cells (or cells of a biological sample) and/or genomic DNA (gDNA) extracted from engineered cells (or from cells of a biological sample), or fragments thereof. In one embodiment, the engineered cells (or cells from a biological sample) comprise genome abnormalities or variants, wherein sequence information regarding the genome abnormality or variant is known. For example, the sequence information corresponding to a first breakpoint and/or a second breakpoint of a chromosomal inversion may be known to the extent that primer and probe molecules can be designed to detect the inversion, e.g., by an amplification method.

In one embodiment, the nucleic acid products generated by the methods described herein comprise sequences that correspond to one or more variants or genome abnormalities. In one other embodiment, the population of cells comprises one or more engineered cells. The engineered cells may be engineered immune cells, e.g., chimeric antigen receptor T (CAR-T) cells. Detection of nucleic acid products (e.g., such as products comprising sequences that correspond to one or more variants or genome abnormalities) can provide important information about cell populations (e.g., cell populations comprising engineered cells, including engineered immune cells) prior to or after their formulation as a drug product, or prior to their administration as part of a drug product to a subject. In addition, the detection of such nucleic acid products in cells from a patient or subject who has been administered the engineered cells (e.g., engineered immune cells) can provide valuable information about the cells during and/or after the treatment regimen. For example, the disclosed methods allow for detection of nucleic acid products during administration of the engineered cells as part of a drug product, and/or subsequent to their administration of the engineered cells as part of a drug product. In addition, detection of variants or genome abnormalities in cell populations from a subject or patient may be performed following administration of a first engineered cell population but before administration of a second engineered cell population. The patient cell populations that are tested may comprise immune cells that are part of a clonal population of the administered engineered cell population, e.g., a clonal population of CAR T cells following in vivo expansion.

In one other aspect, the present disclosure provides assays, methods, systems, kits, and compositions for the analysis, characterization, or screening of one or more donor cell populations prior to any engineering.

In one embodiment, a screening method or assay is provided for two or more different donor cell populations, two or more engineered cell populations, and/or two or more cell populations obtained from a subject or patient who has been administered at least one engineered cell population.

### Sample Preparation

Suitable samples for the methods and assays described herein may be prepared using different protocols. Sample preparation will typically involve any appropriate manipulation of the sample including, without limitation, collection, lyophilization, freezing, extraction, purification, concentration, dilution, or any combination thereof. Sample preparation may further include the addition of a biological sample with one or more assay reagents to form a mixture, e.g., a reaction mixture, and/or running at least one upstream reaction to prepare the biological sample for one or more downstream reactions in the assay. Sample preparation may extract and/or isolate one or more molecular analytes, such as nucleic acid comprising one or more one or more target sequences, and may include further manipulation such as nucleic acid modification and/or nucleic acid fragmentation.

In one embodiment, the methods described herein comprise processing of a cell population to obtain (e.g., extract or isolate) nucleic acid. The processing may comprise any number of different steps and/or techniques, which depends on multiple factors including, without limitation, including the nature of the cell population, the type of nucleic acid of interest, and the type of dPCR method to be used. In general, processing will include nucleic acid purification steps and/or subsequent dilution or concentration steps. Nucleic acid purification methods are well-known in the art and include, without limitation, homogenization, washing, centrifugation, extraction, etc.

A biological sample comprising the cell population may be disupted by different techniques including physical force (e.g. a polytron, grinding or freezing) or chemical methods (e.g. lysis of cells). Homogenation protocols may comprise the use of a detergent or a chaotrope. The extraction of nucleic acid may be achieved by the use of acid phenol/chloroform, filters, glass particles or chromatography (e.g. with appropriate nucleic acids as binding partner). Optionally, the extracted nucleic acid may be further processed to remove contaminants and/or nucleic acids (e.g., nucleic acid molecules which are not of interest and/or might affect the analysis of the nucleic acid molecules of interest). Contaminants may be removed using an enzyme, such as a DNase, an RNase and/or a proteinase, or nucleic acid molecules of interest may be protected with a suitable reagent, such as a DNase inhibitor or an RNase inhibitor.

In one aspect, nucleic acid from a cell population is extracted for analysis. In one embodiment, the extracted nucleic acid (e.g., genomic DNA) is fragmented prior to further processing. In one embodiment, the fragmentation of nucleic acid may comprise random shearing or the use of an enzyme, such as a nuclease or a transposase.

In another aspect, the methods, methods, assays, systems, compositions, and kits for the detection (and/or quantitation or counting) of one or more nucleic acid products may apply to preserved (e.g., fixed) biological samples. Upon removal of a biological sample from its viable environment, e.g., a subject or a cell culture flask, degradation begins immediately. The rate of decay is affected by numerous factors, such as time, temperature, solution buffering conditions, the origin or source of the sample, and physical manipulation (e.g. pipetting, centrifuging). As such, it may be advantageous to stabilize biological samples obtained from an individual patient or subject as described herein. Fixation methods may be used to preserve or stabilize samples immediately following removal from the individual. Different preservation methods for biological samples include, without limitation, dehydration (e.g., methanol), cryopreservation, high salt storage (e.g., using RNAssist, or RNAIater®), and treatment with chemical fixing agents. In general, chemical fixation agents (e.g., paraformaldehyde) create covalent crosslinks in the molecular constituents of the sample. Different techniques for stabilizing biological samples can be used alone or in combination. In one embodiment, a fixed biological samples refers to a fixed cell population, which means cells that have been in contact with a fixative under conditions sufficient to allow or result in the formation of intra- and inter-molecular covalent crosslinks between biomolecules in the biological sample.

### Detection of Nucleic Acid Targets

In other embodiments, biological samples are processed by combining the biological sample with one or more reagents to provide a reaction mixture comprising nucleic acid from the biological sample for the performance of a nucleic acid reaction, e.g., a nucleic acid extension reaction, such as an amplification reaction. The nucleic acid reaction can be specific for one or more target sequences and can report the outcome of the reaction, e.g., whether or not the reaction occurred within a certain range or at a certain threshold. Reaction mixture reagents include, without limitation, primer molecules for nucleic acid targets (e.g., one or more pairs of primer molecules for targets), probe molecules (e.g., one or more probes for one or more sequences, such as a target sequence), one or more enzymes (e.g., polymerases, ligases, reverse transcriptases, restriction enzymes, or any combination thereof), and dNTPs and/or NTPs.

Reaction mixtures, as described herein, may comprise a set of reagents sufficient to perform one or more nucleic acid reactions. The set of reagents may be sufficient to perform a nucleic acid reaction to generate nucleic acid extension products to allow for the detection of one or more nucleic acid target sequences, if present, in the biological sample. Detection of the nucleic acid extension products may be through the use of a probe molecule, as described herein. Probe molecules may comprise a sequence that is complementary to a sequence of the nucleic acid extension product (e.g., a target sequence of the nucleic acid extension product) and a label. Different probe molecules may be present in the reaction mixture to detect different target sequences in different nucleic acid extension products. Alternatively, the same probe molecule may be present in the reaction mixture to detect different nucleic acid extension products having different target sequences. For example, the same reporter molecule may comprise a sequence that is complementary to a sequence of one or more primer molecules used to generate the nucleic acid extension products.

In another aspect, the present disclosure relates to the detection of more than one nucleic acid target or nucleic acid target sequence from nucleic acid molecules of a biological sample from a cell population. Nucleic acid targets or target sequences may be present on single- or double-stranded nucleic acid templates. Targets and/or target sequences may be part of a surrogate molecule, e.g., a nucleic acid molecule (such as a primer molecule), which may be capable of coupling to a nucleic acid template, e.g., via hybridization, and/or may correspond to the target or target sequence. Nucleic acid templates may comprise a target or target sequence that forms at least a portion of or all of the nucleic acid template. In one other embodiment, the target or target sequence corresponds to a nucleic acid extension product (e.g., an amplicon) that is generated by a nucleic acid extension reaction as described herein. Nucleic acid extension products may be partially or completely single- or double-stranded nucleic acid products.

In one embodiment, the more than one nucleic acid targets or nucleic acid target sequences that can be detected include targets or target sequences that correspond to a variant or genome abnormality, such as a chromosome 7 and/or 14 variant or genome abnormality. In other embodiments, the variant or genome abnormality comprises a chromosome inversion with two breakpoints. The methods disclosed herein may comprise detection of a first nucleic acid target or target sequence that corresponds to a first breakpoint of the inversion and/or a second nucleic acid target or target sequence that corresponds to a second breakpoint of the inversion. In another embodiment, the first breakpoint is located at or near the T cell receptor joining gene TRAJ7 (e.g., 14q11) of chromosome 14 and the second breakpoint is located at or near the immunoglobulin heavy chain variable region pseudogene (IGHV3-69-1) (e.g., 14q32) of chromosome 14. In another embodiment, the first breakpoint is located at or near the T cell receptor γ chain gene (e.g., 7p13) of chromosome 7 and the second breakpoint is located at or near the T cell receptor β chain (e.g., 7q35) of chromosome 7.

In one aspect, the instant disclosure relates to the detection of genomic regions of a cell from a cell population that comprises one or more genome abnormalities or variants, such as an inversion that occurred as a result of recombination activating gene-(RAG-) mediated activity. In one embodiment, the site of the inversion is characterized by the presence of one or more consensus RAG recombination signal sequences (RSSs). In one embodiment, the genome abnormality or variant comprise an inversion of a first gene and a second gene, wherein the first gene and the second gene each comprise an adjacent consensus RSS. In one other embodiment, the first gene is TRAJ7 which comprises an adjacent RSS-12 sequence and the second gene is IGHV3-69-1 which comprises an adjacent RSS-23 sequence (see the 12/23 rule-van Gent et al. The RAG1 and RAG2 Proteins Establish the 12/23 Rule in V (D) J Recombination, Cell 85 (1), 107-113, 10.1016/s0092-8674 (00) 81086-7 (1996)). In one embodiment, consensus RSSs are typically comprised of a conserved heptamer (consensus 5′-CACAGTG-3′) and nonamer (consensus 5′-ACAAAAACC-3′) sequence separated by either 12 (RSS-12) or 23 (RSS-23) nucleotides of variable sequence. Exemplary RSSs are depicted below.

(SEQ ID NO: 15)
CACAGTGX_{A}CACAGTGX_{A}CACAGTGX_{A}CACAGTGX_{A}CACAGTGX_{A}C
ACAGTGX_{A}CACAGTGX_{A},
wherein X_{A} = 12 to 23 nucleotides.
(SEQ ID NO: 16)
ACAAAAACCX_{A}ACAAAAACCX_{A}ACAAAAACCX_{A}ACAAAAACCX_{A}AC
AAAAACCX_{A}ACAAAAACCX_{A}ACAAAAACCX_{A}ACAAAAACCX_{A}ACAA
AAACCX_{A},
wherein X_{A} = 12 to 23 nucleotides

As discussed in Example 1, a chromosome 14 inversion was detected in a patient and consensus RAG recombination signal sequences (RSSs) were observed at both sites of inversion. Without being bound by any theory, the presence of RSS at both ends of inversion supports the hypothesis that the initial inversion was a consequence of RAG mediated V (D) J recombination between the 2 distal sites. It is known that TRAJ7 harbors an adjacent RSS-12 sequence and IGHV3-69-1 contains an adjacent RSS-23 sequence, both conforming to the 12/23 criterion. Deletions following the breaks and prior to ligation at TRAV38-2DV8 and TRAJ7, as well as at IGHV3-42 and IGHV3-69-1 were also observed. It has been observed that the insertion of the signal end fragment generated by the RAG proteins nearly always produces deletions or other rearrangements of target DNA (Chatterji, M. et al., Mobilization of RAG-generated signal ends by transposition and insertion in vivo. Mol Cell Biol 26, 1558-1568, doi: 10.1128/MCB.26.4.1558-1568.2006 (2006)).

The chromosome 14 inversion has been detected in post-thymic, mature T cells of normal individuals and is believed to be mediated by V (D) J recombination machinery (Callen, E. et al. Chimeric IgH-TCRalpha/delta translocations in T lymphocytes mediated by RAG. Cell Cycle 8, 2408-2412, doi: 10.4161/cc.8.15.9085 (2009); Machado, H. E. et al. Genome-wide mutational signatures of immunological diversification in normal lymphocytes. bioRxiv, 2021.2004.2029.441939, doi: 10.1101/2021.04.29.441939 (2021); Aurias, A. et al. Inversion (14) (q12qter) or (q11.2q32.3): the most frequently acquired rearrangement in lymphocytes. Hum Genet 71, 19-21, doi: 10.1007/BF00295660 (1985)). In addition, chromosomal breakpoints involving 14q11 and/or 14q32 have been detected (Hecht, F. et al. Fragile sites limited to lymphocytes: molecular recombination and malignancy. Cancer Genet Cytogenet 26, 95-104, doi: 10.1016/0165-4608 (87) 90137-3 (1987)). Studies examining the integrity of chromosomes in normal cultured lymphocytes have shown 4 frequent sites of chromosome breakage: 7p13 (near location of TCRγ chain), 7q35 (location of TCRβ chain), 14q11 (location of TCRα chain) and 14q32 (location of IGH chain) and suggest that these act as fragile sites specifically in normal lymphocytes as defined by the non-random frequency of chromosomal translocations or inversions associated with them (Hecht, F. (1987); Welch, J. P. et al. Non-random occurrence of 7-14 translocations in human lymphocyte cultures. Nature 255, 241-245, doi: 10.1038/255241a0 (1975)).

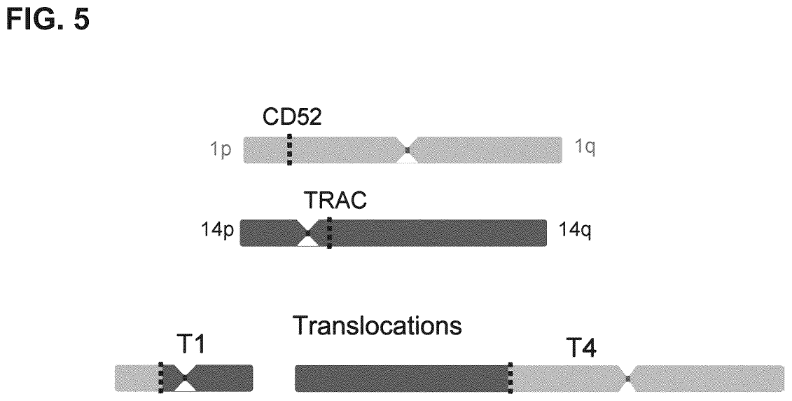

In another embodiment, the more than one nucleic acid targets or nucleic acid target sequences that can be detected further includes a target or target sequence that corresponds to a gene-edited genomic region that comprises a variant or genome abnormality. The gene-edited region may be a region of chromosome 14 and/or chromosome 1. The variant of the gene-edited region of chromosome 14 may be a translocation at the break in a gene from chromosome 14 to another chromosome, such as chromosome 1. In one embodiment, the translocation is at the break in T Cell Receptor Alpha Constant (TRAC) gene from chromosome 14 to a gene from chromosome 1, e.g., the CD52 gene. In another embodiment, the translocation is at the break in the CD52 gene from chromosome 1 to a gene from chromosome 14, e.g., the TRAC gene (see FIG. 5). In one embodiment, the TRAC/CD52 translocation is a t (1; 14) (p36; q32) translocation.

In another embodiment, the gene-edited region may be a region of chromosome 14 and/or chromosome 15. The variant of the gene-edited region of chromosome 14 may be a translocation at the break in a gene from chromosome 14 to another chromosome, such as chromosome 15. In one embodiment, the translocation is at the break in T Cell Receptor Alpha Constant (TRAC) gene from chromosome 14 to a gene from chromosome 15, e.g., the beta-2 microglobulin (β2M) gene. In another embodiment, the translocation is at the break in the β2M gene from chromosome 15 to a gene from chromosome 14, e.g., the TRAC gene.

In another embodiment, the gene-edited region may be a region of chromosome 19 and chromosome 14 or 15. The variant of the gene-edited region of chromosome 19 may be a translocation at the break in a gene from chromosome 19 to another chromosome, such as chromosome 14 or 15. In one embodiment, the translocation is at the break in the CD70 gene from chromosome 19 to a gene from chromosome 14, e.g., the TRAC gene from chromosome 14. In another embodiment, the translocation is at the break in the CD70 gene from chromosome 19 to a gene from chromosome 15, e.g., the β2M gene from chromosome 15.

In a further embodiment, the gene-edited region may be a region of chromosome 15 and chromosome 16. The variant of the gene-edited region of chromosome 15 may be a translocation at the break in a gene from chromosome 15 to another chromosome, such as chromosome 16. In one embodiment, the translocation is at the break in the β2M gene from chromosome 15 to a gene from chromosome 16, e.g., the Class II Major Histocompatibility Complex Transactivator (CIITA) gene on chromosome 16.

In another embodiment, the method for detecting one or more nucleic acid targets or nucleic acid target sequences further includes detection of a control sequence. The control sequence may be a sequence from the human ribonuclease P protein subunit p30 (RPP30) gene.

In one aspect, the present invention concerns nucleic acid-based (e.g., digital PCR, such as droplet digital PCR or ddPCR) methods for the detection and/or quantification of one or more variants or genome abnormalities in a sample through the use of an array of reaction mixtures or a plurality of partitions. Extension products, e.g., amplicons, generated in a dPCR method can be used to determine the presence of a genome abnormality or variant in a cell population. In one embodiment, the extension product may be between about 100 bp to about 1000 bp, between about 100 bp to about 950 bp, between about 100 bp to about 900 bp, between about 100 bp to about 850 bp, between about 100 bp to about 800 bp, between about 100 bp to about 750 bp, between about 100 bp to about 700 bp, between about 100 bp to about 650 bp, between about 100 bp to about 600 bp, between about 550 bp to about 500 bp, between about 100 bp to about 450 bp, between about 100 bp to about 400 bp, between about 100 bp to about 350 bp, between about 100 bp to about 300 bp, between about 100 bp to about 200 bp, or between about 100 bp to about 150 bp. In other embodiments, the extension product is about 100 bp, about 150 bp, about 200 bp, about 250 bp, about 300 bp, about 350 bp, about 400 bp, about 550 bp, about 600 bp, about 650 bp, about 700 bp, about 750 bp, about 800 bp, about 850 bp, about 900 bp, about 950 bp, or about 1000 bp.

dPCR is an improved method based on conventional PCR methods that advantageously allows a user to directly quantify and clonally amplify different species of nucleic acid including DNA, cDNA, RNA or combinations thereof. Importantly, dPCR methods of quantifying the amount of nucleic acids is far more precise and accurate as compared to conventional PCR (e.g., quantitative PCR or qPCR). dPCR is performed in a single reaction within a biological sample but the biological sample is separated into a large number of individual partitions or reaction mixtures, where individual reactions can occur. Methods using separate partitions or reaction mixtures are not only more reliable and more sensitive but also allow for more accurate quantification. The distribution of a dPCR sample such that individual nucleic acid molecules are present in a plurality of partitions or reaction mixtures allows a user to estimate the number of nucleic acid molecules by assuming that the molecules follow a Poisson distribution. Each partition or reaction mixture will contain a negative or positive reaction (e.g., “0” or “1”, respectively). dPCR experimental results are plotted on a graph of fluorescence intensity versus droplet number. Based on a fluorescence intensity threhold, all positive droplets are scored positive (e.g., above the threshold) and assigned a value of 1 while all negative droplets are scored negative (e.g., below the threshold) and assigned a value of 0. Following an amplification reaction, nucleic acid molecules may be quantified based on a counting of each individual partition or reaction mixture that contains an end-product of a positive amplification reaction. While qPCR quantitation may depend on the PCR amplification efficiency, dPCR does not thereby avoiding reliance on potentially uncertain exponential data for quantification of a target nucleic acid.

In conventional quantitative PCR, the quantitation result may depend on the amplification efficiency of the PCR process. dPCR, however, is not dependent on the number of amplification cycles to determine the initial sample amount, which means less reliance on uncertain exponential data to quantify target nucleic acids, thereby providing absolute quantification.

Digital PCR, e.g., droplet digital PCR, allows for the multiplexed detection of numerous targets simultaneously in different ways including, without limitation, amplitude multiplexing and probe-mixing multiplexing. For amplitude multiplexing, the concentrations of probe molecules and/or primer molecules can be manipulated such that positive droplets are distinguishable on a two-dimensional (2-D) plot. Probe-mixing multiplexing involves the detection of unique signals (e.g., flourescent signals) based on varying ratios of a probe in at least two different fluorescent configurations. For example, two probes having different detectable moieties, e.g., one probe with fluorescein (FAM) and another probe with hexachlorofluorescein (HEX), can be used in the same assay for two different targets or target sequences. The amplification of two different targets can be viewed as a 2-D plot in which FAM fluorescence is plotted versus HEX fluorescence for each droplet, which can be in one of four groups: FAM+/HEX−; FAM+/HEX+; FAM−/HEX+, or FAM−/HEX−. Software may be used to fit the fraction of positive droplets to a Poisson distribution to determine the absolute start copy number in units of copies/ul of a sample and report the target DNA concentration as copies per ul in the sample (QuantaSoft software from Bio-Rad). When compared to amplitudie-mixing multiplexing, probe-mixing multiplexing may be more suitable for the detection of rare mutations or low-concentation samples. In one embodiment, different detectable moieties may be used to label nucleic acid probes including, without limitation, 5,6-FAM: 5- and 6-carboxyfluorescein; 6-FAM: 6-carboxyfluorescein; JOE: 6-carboxy-4′-, 5′-dichloro-2′-, 7′-dimethoxy-fluorescein (JOE); 4,7,2′,4′,5′,7′-hexachloro-6-carboxy-fluorescein (HEX); 4,7,2′,7′-tetrachloro-6-carboxy-fluorescein (TET); 6-carboxytetramethyl-rhodamine (TAMRA); 2′-chloro-7′phenyl-1,4-dichloro-6-carboxy-fluorescein (VIC); 2′-chloro-5′-fluoro-7′,8′-benzo-1,4-dichloro-6-carboxyfluorescein (NED); 5- and 6-carboxy-X-rhodamin (ROX); or any combination thereof.

### Cell Population Analysis

In an additional aspect, the present disclosure relates to the analysis of different cell populations. In one embodiment, the analysis includes a screening method for the detection and/or quantitation of a variant or genome abnormality in a cell population using an assay comprising a digital polymerase chain reaction (dPCR), e.g., a droplet dPCR (ddPCR) reaction. As discussed herein, methods to understand the degree of variants or genome abnormalities in a cell population are valuable independent of the cause, e.g., caused or not caused by gene editing off-target effects. It may not only be desirable to clarify whether or not a variant or genome abnormality is present in cells of a biological sample, but it may also be required to quantify with high degree of precision and accuracy the frequency of occurrence of the variant or genome abnormality (e.g., structural variant, such as a chromosomal structural variant) in the biological sample. Biological samples may be comprise nucleic acid from a cell population (e.g., an engineered cell population and/or a cell population derived from a subject previously administered an engineered cell population). Screening methods of interest include, without limitation, screening of (i) donor cells prior to modification or engineering (e.g., by gene-editing), (ii) engineered cells (e.g., modified donor cells) that comprise one or more genomic regions that have been modified, and/or (iii) cells derived from a biological sample obtained from a patient who has been treated with a cell-based therapeutic composition, e.g., a composition comprising the engineered cells.

In one aspect, the present invention concerns nucleic acid-based (e.g., dPCR) methods for the detection and/or quantification of one or more variants or genome abnormalities in a sample through the use of an array of reaction mixtures or a plurality of partitions. In one embodiment, the method comprises providing a cell population that includes cells containing engineered cells, such as genetically modified cells, e.g., genetically modified immune cells. The genetically modified cells may comprise one or more genomic regions that have been subjected to gene-editing.

In addition, the engineered cells or cells from a patient or subject may be suspected of comprising or comprises one or more variants or genome abnormalities or may comprise the one or more variants or genome abnormalities. In one embodiment, the one or more variants or genome abnormalities are known. For example, the sequence information corresponding to a first breakpoint and/or a second breakpoint of a chromosomal translocation may be known to the extent that primer and probe molecules can be designed to detect the translocation, e.g., by an amplification method. In other embodiments, the variants or genome abnormalities may be due to the genetic modification process (e.g., gene editing) or may be present independent of the genetic modification process. In another embodiment, the one or more variants or genome abnormalities are present in chromosome 14. In an alternative embodiment, the engineered cells comprise a chromosome 14 that comprises one or more variants or genome abnormalities.

In one additional embodiment, the method comprises providing a cell population that includes immune cells from a biological sample obtained from a patient. The patient may be in the process of being treated with or may have been previously treated with an engineered cell drug product, e.g., a CAR T cell drug product. The immune cells from the biological sample may comprise immune cells that have originated from an engineered cell that was previously administered to the patient. For example, the immune cells may be part of a clonal population of the administered engineered cell, e.g., a clonal population of an administered CAR T cell population following in vivo expansion. The identity of such clones can be determined with such methods as T cell receptor beta (TCRB) repetoire sequencing and single cell methods, as described herein. The immune cells may comprise (i) one or more genomic regions that correspond to region that were subjected to gene-editing in the engineered cell and/or (ii) one or more variants or genome abnormalities. In one embodiment, the one or more variants or genome abnormalities are known. For example, the sequence information corresponding to the variant or genome abnormality may be known to the extent that primer and probe molecules can be designed to detect the variant, e.g., by an amplification method. In other embodiments, the variants or genome abnormalities may be present due to the genetic modification process (e.g., gene editing) of the engineered cell or may be present independent of the genetic modification process of the engineered cell, e.g., present due to a spontaneous event, such as an event during in vivo expansion. In another embodiment, the one or more variants or genome abnormalities are present in chromosome 14. In an alternative embodiment, the engineered cells comprise a chromosome 14 that comprises one or more variants genome abnormalities.

In a further embodiment, the instant disclosure provides methods for detecting a variant or genome abnormality in engineered immune cells using genomic deoxyribonucleic acid (gDNA) from a population of cells comprising an engineered immune cell. The engineered immune cell has a chromosome 14 that (i) is suspected of containing or contains a variant or genome abnormality and (ii) contains an edited genomic region. One or more of the gDNA molecules from the engineered immune cell can be tested (as per the methods described herein) to determine whether it comprises the variant or genome abnormality. In a further embodiment, the method comprises subjecting the gDNA molecule to conditions sufficient to generate a nucleic acid extension product. The nucleic acid extension product includes a sequence corresponding to the variant or genome abnormality or a complement thereof. The method may further comprise detecting the nucleic acid extension product as an indicator of the presence of the variant or genome abnormality in the engineered immune cell.

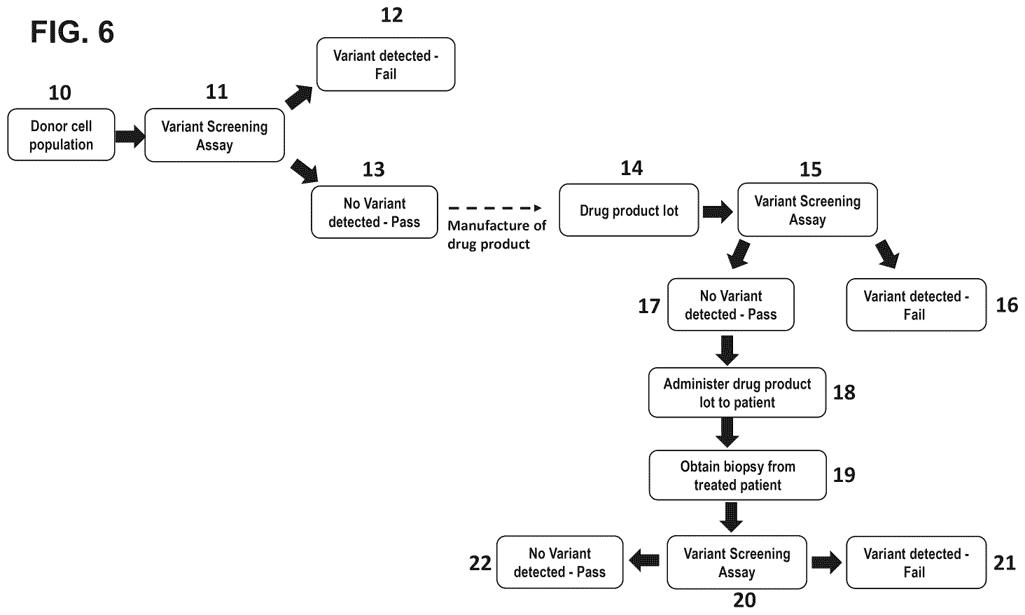

In one aspect, the screening method is quantitative and/or qualitative method. In one embodiment, the quantitative screening method is a nucleic acid extension assay. In one aspect, the methods, assays, systems, compositions, and kits described herein may be used at different stages of manufacturing of an engineered cell population and/or monitoring of a patient or subject who has been administered the engineered cell population. As shown in FIG. 6, a donor cell population 10 may be screened for a variant or genome abnormality 11 to inform a decision on whether to proceed with one or more modifications of the donor cell population, such as those described herein for the manufacture of engineered immune cells, e.g., CAR T cells. If the variant is detected 12, then the donor cell population may be considered to have failed the screening assay. If the variant is not detected 13, then the donor cell population may be considered to have passed the screening assay and can be moved forward in the drug product manufacturing process.

As further shown in FIG. 6, a drug product lot 14 comprising engineered cells may be screened for a variant or genome abnormality 15 to inform a decision whether to advance a drug lot product to administration to a patient or subject. If the variant is detected 16, then the drug product lot may be considered to have failed the screening assay. If the variant is not detected 17, then the drug product lot may be considered to have passed the screening assay and can be moved forward as a drug product lot suitable for administration 18 to a patient or a subject.

In one embodiment, the drug product lot 14 may comprise engineered cells derived from a donor cell population. In another embodiment, the donor cell population may have previously been screened for a variant or genome abnormalilty, e.g., step 11 of FIG. 6. In some embodiments, the donor cell population may be obtained from a subject who will ultimately receive an engineered cell population derived from the donor cell population. For example, the donor cell population may comprise autologous immune cells obtained from a subject who will ultimately receive CAR T cells derived from the donor cell population. In other embodiments, the donor cell population may be obtained from a donor, who is a different individual than the subject who will receive an engineered cell population that is derived from the donor cell population. For instance, the donor cell population may comprise allogeneic immune cells obtained from a donor, who is a different individual than the subject who will receive CAR T cells derived from the donor cell population.

Screening of one or more drug product lots and cells from a patient or subject that has been administered the one or more drug product lots can provide valuable information about the circumstances under which a variant or genome abnormality arises. As further shown in FIG. 6, a biopsy 19 comprising a biological sample may be screened for a variant or genome abnormality 20 in one or more cells from the patient or subject. Where a variant or genome abnormality is not detected 17 in the drug product lot and subsequently administered to a patient but is detected 21 in cells from the same patient following treatment, then this may indicate that the variant arose in vivo following administration.

In one embodiment, the one or more cells from the patient or subject may be immune cells that are part of a clonal population of the engineered cell population that was administered, e.g., a clonal population of CAR T cells following in vivo expansion. In one embodiment, the variant is detected 21 (see FIG. 6) in the engineered immune cells, e.g., CAR T cells, that were previously adminstered to the patient and/or in the clonal population of immune cells that arose following administration, e.g., a clonal population of CAR T cells following in vivo expansion.

In one aspect, the present disclosure provides methods for detecting a variant or genome abnormality over the course of treatment of a patient or a subject. In one embodiment, the method comprises generating a drug product that comprises an engineered immune cell population (e.g., CAR T cell population) wherein the cell population comprises edited genomes. In a further embodiment, the engineered immune cell population was derived from a donor cell population. In another embodiment, the method further comprises administering the drug product to a patient or a subject in need. In one other embodiment, the method comprises obtaining a biopsy from the patient, wherein the biopsy comprises one or more of the engineered immune cells or immune cells that are part of a clonal population of the engineered immune cell population that was administered. The method can further comprise detecting a variant or genome abnormality in one or more cells from the biopsy. In one embodiment, the method further comprises detecting the variant or genome abnormality in engineered immune cells from the drug product lot and/or in the donor cell population.

Drug product lots and/or cells from a patient may be subjected to other screening methods prior to screening for genome abnormalities or variants. For instance, cells may be subjected to G-banding (or Giemsa banding), a cytogenetic technique used to produce a visible karyotype by staining condensed chromosomes. Cells may also be subjected to fluorescence in situ hybridization (FISH), which allows for the visualization and mapping the genetic material in an individual's cells, including specific genes or portions of genes. A FISH assay utilizes probes with attached fluorescent reporter molecules that are capable of annealing to specific target sequences of nucleic acid from the cells in order to confirm the presence or absence of a particular genetic abnormality or variant when viewed under fluorescence microscopy. If the presence of a genome abnormality or variant is suspected based on the results of the G-banding or FISH assay results, then the cells may be screened for one or more genome abnormalities or variants.

In addition, drug product lots and/or cells from a patient may be subjected to a cell proliferation assay. For example, an IL-2 independent proliferation assay can be used to assess any aberrant proliferation of the engineered cells of a drug product lot, e.g., CAR T cells. In the absence of target activation of CAR T cells and in the absence of IL-2, CAR T cells are not expected to proliferate. In one embodiment, the sensitivity of the cell proliferation assay is about 1 aberrant cell in about 100,000 cells.

In one aspect, the present disclosure provides a method for detecting a genome abnormality in an engineered cell population. In one embodiment, the method comprises providing genomic deoxyribonucleic acid (gDNA) molecules from an engineered cell population, wherein the engineered cell population is suspected of comprising a chromosome 14 inversion characterized by a centromeric inversion site at the T cell receptor alpha/delta locus (TCR A/D) and a telomeric inversion site at the immunoglobulin heavy chain (IGH) variable region. In another embodiment, the engineered cell population further comprises an edited genomic region. In a further embodiment, the method comprises the step of subjecting a gDNA molecule to conditions sufficient to generate a nucleic acid extension product, wherein the nucleic acid extension product comprises a sequence corresponding to the chromosome 14 inversion or a complement thereof. In one other embodiment, the generating of the nucleic acid extension product comprises nucleic acid extension with primer molecules. In one embodiment, the primer molecules comprise a pair of primer molecules. In another embodiment, the pair of primer molecules comprises SEQ ID NO: 1 and 2, wherein the pair of primer molecules corresponds to the telomeric inversion site. In another embodiment, the pair of primer molecules comprises SEQ ID NO: 4 and 5, wherein the pair of primer molecules corresponds to the centromeric inversion site. In a further embodiment, the method comprises the step of detecting the nucleic acid extension product as an indicator of the presence of the chromosome 14 inversion in the engineered cell population. In one embodiment, the detecting comprises use of a probe molecule specific for a nucleic acid extension product. The nucleic acid extension product may correspond to the telomeric inversion site, wherein the probe molecule comprises SEQ ID NO: 3. Alternatively, the nucleic acid extension product may correspond to the centromeric inversion site, wherein the probe molecule comprises SEQ ID NO: 6.

### Single Cell Methods for the Detection of Nucleic Acid Products

The engineering of cells often utilizes different genetic modification methods, including gene editing technologies, as further described herein. Off-target effects in gene editing are unwanted and present significant risks in the development of clinical cell therapy products. While bulk next-generation sequencing techniques are valuable tools for the identification of potential off-target effects, the genetic heterogeneity of a cell population at the single cell level may be obscured by an average readout provided by a bulk measurement. Rare events within and across the population of cells might not be detected with such average signals. Accordingly, the detection of one or more variants or genome abnormalities in one or more cells on a single cell level may be advantageous. In one embodiment, one or more of single cell RNA sequencing (scRNA-seq), single cell DNA sequencing (scDNA-seq), Assay for Transposase-Accessible Chromatin (scATAC-seq), protein-labeling based single cell techniques (e.g., CITE-seq and REAP-seq), and combinations thereof, may be used. Different single cell analysis platforms may used used including droplet-in-an-emulsion approaches (e.g., Chromium instrument—10× Genomics, Tapestri instrument—Mission Bio) and well-based approaches (e.g., BD Rhapsody—Becton Dickinson, Singleron Matrix—Singleron Biotechnologies).

In another aspect, the present disclosure provides methods, compositions, systems, and kits for the generation of one or more barcoded nucleic acid molecules from a population of cells that comprises an engineered cell, which is suspected of comprising or comprises one or more variants or genome abnormalities. In some embodiments, the one or more barcoded nucleic acid molecules correspond to a nucleic acid or protein component of a single cell, e.g., a single engineered cell, of the population of cells. In one embodiment, the one or more barcoded nucleic acid molecules comprise a barcode sequence corresponding to the single cell and a sequence corresponding to a region of genomic deoxyribonucleic acid (gDNA). The region of gDNA may comprise the one or more variants or genome abnormalities. The one or more barcoded nucleic acid molecules may further comprise a barcode sequence corresponding to the single cell and a sequence corresponding to a protein component of the single cell. The protein component may comprise an extracellular surface protein, an intracellular protein, or a protein secreted from the single cell. In one embodiment, the protein component comprises a one or more regions of a chimeric antigen receptor (CAR) including, without limitation, an antigen binding domain (e.g., an scFV domain), a transmembrane domain, and an intracellular domain (e.g., a domain comprising an immune cell activation domain), as further described herein. In another embodiment, the CAR is part of the engineered immune cell from the population of cells. In other embodiments, the single cell, e.g., a single engineered cell, of the population of cells comprises a protein labeling agent with a nucleic acid reporter molecule. The nucleic acid reporter molecule may comprise a nucleic acid sequence that identifies or corresponds to the protein labeling agent and/or the target of the protein labeling agent.

In a further embodiment, the one or more barcoded nucleic acid molecules may further comprise a barcode sequence corresponding to the single cell and a sequence corresponding to a ribonucleic acid (RNA) sequence (e.g., a messenger RNA or mRNA transcript) of the single cell. In one other embodiment, the RNA sequence corresponds to a sequence corresponding to a region of genomic deoxyribonucleic acid (gDNA). The region of gDNA may comprise the one or more variants or genome abnormalities.

In other embodiments, the one or more barcoded nucleic acid molecules may comprise a barcode sequence corresponding to the single cell and a sequence corresponding to (or indicative of) a region of accessible (or open) chromatin of the single cell. The region of accessible (or open) chromatin may comprise the one or more variants or genome abnormalities. In an additional embodiment, the one or more barcoded nucleic acid molecules further comprises (i) a barcode sequence corresponding to the single cell and a sequence corresponding to a ribonucleic acid (RNA) sequence (e.g., a messenger RNA or mRNA transcript) of the single cell and/or (ii) a barcode sequence corresponding to the single cell and a sequence corresponding to a protein component of the single cell.

In an additional embodiment, the one or more barcoded nucleic acid molecules may comprise a barcode sequence corresponding to the single cell and a sequence corresponding to a region of genomic DNA. Genomic DNA (gDNA) from a cell, e.g., an engineered cell, having or suspected of having a genome abnormality may be processed using single cell methods. In one embodiment, the method includes providing a single cell lysate comprising gDNA from the engineered cell, such as an engineered immune cell. The single cell lysate is then sequestered or partitioned into a partition with a primer molecule that targets a region corresponding to the genome abnormality and a nucleic acid barcode molecule that comprises a barcode sequence and a capture sequence. In another embodiment, the method further includes the step of generating a barcoded nucleic acid molecule comprising the barcode sequence or a complement thereof and a sequence corresponding to the region or a complement thereof.

In another embodiment, the generating step comprises use of the primer molecule and gDNA to generate a nucleic acid product. The nucleic acid product may comprise a sequence complementary to the capture sequence. In one other embodiment, the generating step comprises hybridization of the nucleic acid barcode molecule to the nucleic acid product via the sequence complementary to the capture sequence. In some embodiments, the generating step further comprises an extension of the sequence complementary to the capture sequence to generate the barcoded nucleic acid molecule. The generating step may include the use of an additional primer molecule to generate the nucleic acid product. The extension may further comprise the use of an additional primer molecule to generate the barcoded nucleic acid molecule. In other embodiments, the generating step comprises nucleic acid amplification.

In some embodiments, the partition is a droplet or a well and/or the partition is among a plurality of partitions. In one embodiment, the barcode sequence is specific to the partition. In other embodiments, the genome abnormality is at chromosome 14. The engineered cell, e.g., an engineered immune cell, may be a chimeric antigen receptor T (CAR-T) cell. In addition, the population of cells may further comprise an additional engineered immune cell that is free of the genome abnormality. In an additional embodiment, the population of cells is analyzed prior to administration to a subject. In another embodiment, the population of cells originate from a biological sample obtained from a subject that was previously administered the engineered cell, including an engineered immune cell. In one embodiment, the biological sample is a bone marrow sample or a peripheral blood sample. The engineered cell may have been derived from a donor immune cell. In one embodiment, the sample was obtained between about 5 days and about 60 days after administration of the engineered cell, including engineered immune cells.

### Immune Cells

Cells suitable for use with the methods and/or reagents described herein include immune cells. Prior to the in vitro manipulation or genetic modification (e.g., as described herein), cells for use in methods described herein (e.g., immune cells) can be obtained from a subject. Cells can be obtained from a number of non-limiting sources, including peripheral blood mononuclear cells, bone marrow, lymph node tissue, cord blood, thymus tissue, stem cell- or iPSC-derived immune cells, tissue from a site of infection, ascites, pleural effusion, spleen tissue, and tumors. In some embodiments, any number of T cell lines available and known to those skilled in the art, can be used. In some embodiments, cells can be derived from a healthy donor, from a patient diagnosed with cancer or from a patient diagnosed with an infection. In some embodiments, cells can be part of a mixed population of cells which present different phenotypic characteristics.

In some embodiments, immune cells are autologous immune cells obtained from a subject who will ultimately receive the engineered immune cells. In some embodiments, immune cells are allogeneic immune cells obtained from a donor, who is a different individual than the subject who will receive the engineered immune cells.

In some embodiments, immune cells comprise T cells. T cells can be obtained from a number of sources, including peripheral blood mononuclear cells (PBMCs), bone marrow, lymph nodes tissue, cord blood, thymus tissue, stem cell- or iPSC-derived T cells, tissue from a site of infection, ascites, pleural effusion, spleen tissue, and tumors. In some embodiments, T cells can be obtained from a volume of blood collected from the subject using any number of techniques known to the skilled person, such as FICOLL™ separation.

Cells can be obtained from the circulating blood of an individual by apheresis. The apheresis product typically contains lymphocytes, including T cells, monocytes, granulocytes, B cells, other nucleated white blood cells, red blood cells, and platelets. In some embodiments, the cells collected by apheresis can be washed to remove the plasma fraction, and placed in an appropriate buffer or media for subsequent processing.

PBMCs can be used directly for genetic modification with the immune cells (such as CARs or TCRs) using methods as described herein. In certain embodiments, after isolating the PBMCs, T lymphocytes can be further isolated and both cytotoxic and helper T lymphocytes can be sorted into naive, memory, and effector T cell subpopulations either before or after genetic modification and/or expansion.

In certain embodiments, T cells are isolated from PBMCs by lysing the red blood cells and depleting the monocytes, for example, using centrifugation through a PERCOLL™ gradient. A specific subpopulation of T cells, such as CCR7+, CD95+, CD122, CD27+, CD69+, CD127+, CD28+, CD3+, CD4+, CD8+, CD25+, CD62L+, CD45RA+, and CD45RO+ T cells can be further isolated by positive or negative selection techniques known in the art. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. Flow cytometry and cell sorting can also be used to isolate cell populations of interest for use in the present disclosure.

In some embodiments, a population of T cells is enriched for CD4+ cells.

In some embodiments, a population of T cells is enriched for CD8+ cells.

In some embodiments, CD8+ cells are further sorted into naive, central memory, and effector cells by identifying cell surface antigens that are associated with each of these types of cells. In some embodiments the expression of phenotypic markers for naïve T cells include CD45RA+, CD95−, IL2RB−, CCR7+, and CD62L+. In some embodiments the expression of phenotypic markers for stem cell memory T cells include CD45RA+, CD95+, IL2RB+, CCR7+, and CD62L+. In some embodiments the expression of phenotypic markers for central memory T cells include CD45RO+, CD95+, IL2Rβ+, CCR7+, and CD62L+. In some embodiments the expression of phenotypic markers for effector memory T cells include CD45RO+, CD95+, IL2RB+, CCR7−, and CD62L−. In some embodiments the expression of phenotypic markers for T effector cells include CD45RA+, CD95+, IL2RB+, CCR7−, and CD62L−. Thus, CD4+ and/or CD8+T helper cells can be sorted into naive, stem cell memory, central memory, effector memory and T effector cells by identifying cell populations that have cell surface antigens.

It will be appreciated that PBMCs can further include other cytotoxic lymphocytes such as NK cells or NKT cells. An expression vector carrying the coding sequence of a chimeric receptor as disclosed herein can be introduced into a population of human donor T cells, NK cells or NKT cells. Standard procedures are used for cryopreservation of T cells expressing the CAR for storage and/or preparation for use in a human subject. In one embodiment, the in vitro transduction, culture and/or expansion of T cells are performed in the absence of non-human animal derived products such as fetal calf serum and fetal bovine serum. In various embodiments a crypreservative media can comprise, for example, CryoStor® CS2, CS5, or CS10 or other medium comprising DMSO, or a medium that does not comprise DMSO.

### Engineered Immune Cells

Provided herein are engineered immune cells expressing the CARs of the disclosure (e.g., CAR-T cells).

In some embodiments, an engineered immune cell comprises a population of CARs, each CAR comprising extracellular antigen-binding domains. In some embodiments, an engineered immune cell comprises a population of CARs, each CAR comprising different extracellular antigen-binding domains. In some embodiments, an immune cell comprises a population of CARs, each CAR comprising the same extracellular antigen-binding domains.

The engineered immune cells can be allogeneic or autologous.

In some embodiments, the engineered immune cell is a T cell (e.g., inflammatory T-lymphocyte, cytotoxic T-lymphocyte, regulatory T-lymphocyte, helper T-lymphocyte, or tumor infiltrating lymphocyte (TIL)), NK cell, NK-T-cell, TCR-expressing cell, dendritic cell, killer dendritic cell, a mast cell, or a B-cell. In some embodiments, the cell can be derived from the group consisting of CD4+T-lymphocytes and CD8+T-lymphocytes. In some exemplary embodiments, the engineered immune cell is a T cell. In some exemplary embodiments, the engineered immune cell is an alpha beta T cell. In some exemplary embodiments, the engineered immune cell is a gamma delta T cell. In some exemplary embodiments, the engineered immune cell is a macrophage. In some embodiments, the engineered immune cells are human cells.

In some embodiments, the engineered immune cell can be derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells (iPSC), totipotent stem cells or hematopoietic stem cells. Stem cells can be CD34+ or CD34−.

In some embodiments, the cell is obtained or prepared from peripheral blood. In some embodiments, the cell is obtained or prepared from peripheral blood mononuclear cells (PBMCs). In some embodiments, the cell is obtained or prepared from bone marrow. In some embodiments, the cell is obtained or prepared from umbilical cord blood. In some embodiments, the cell is a human cell. In some embodiments, the cell is transfected or transduced by the nucleic acid vector using a method selected from the group consisting of electroporation, sonoporation, biolistics (e.g., Gene Gun), transfection, lipid transfection, polymer transfection, nanoparticles, viral transduction or viral transfection (e.g., retrovirus, lentivirus, AAV) or polyplexes. In some embodiments the cell is a T cell that has been re-programmed from a non-T cell. In some embodiments the cell is a T cell that has been re-programmed from a T cell.

### Binding Agents

In embodiments, the disclosed methods comprise the use of an antibody or antigen binding agent (e.g., comprising an antigen binding domain or comprising an antibody or fragment thereof). As discussed below, in various embodiments engineered immune cells can also comprise a binding agent.

As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CHI, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain. Those skilled in the art are well familiar with antibody structure and sequence elements, recognize “variable” and “constant” regions in provided sequences, and understand that there may be some flexibility in definition of a “boundary” between such domains such that different presentations of the same antibody chain sequence may, for example, indicate such a boundary at a location that is shifted one or a few residues relative to a different presentation of the same antibody chain sequence.

Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation.

For purposes of the instant disclosure, in certain embodiments, any polypeptide or complex of polypeptides that includes sufficient immunoglobulin domain sequences as found in natural antibodies can be referred to and/or used as an “antibody,” whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art.

Moreover, the term “antibody” as used herein, can refer to any of the art-known or developed constructs or formats for utilizing antibody structural and functional features in alternative presentation. For example, in some embodiments, an antibody utilized in the methods of the instant disclosure is in a format selected from, but not limited to, intact IgA, IgG, IgE or IgM antibodies; bi- or multi-specific antibodies (e.g., Zybodies®, etc.); antibody fragments such as Fab fragments, Fab fragments, F (ab) 2 fragments, Fd fragments, and isolated CDRs or sets thereof; single chain variable fragments (scFVs); polypeptide-Fc fusions; single domain antibodies (e.g., shark single domain antibodies such as IgNAR or fragments thereof); camelid antibodies (also referred to herein as nanobodies or VHHs); shark antibodies, masked antibodies (e.g., Probodies®); Small Modular ImmunoPharmaceuticals (SMIPs™); single chain or Tandem diabodies (TandAb®); VHHs; Anticalins®; Nanobodies® minibodies; BiTE®s; ankyrin repeat proteins or DARPINS®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.).

As used herein, the term “antibody agent” generally refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc. as is known in the art. In many embodiments, the term “antibody agent” is used to refer to one or more of the art-known or developed constructs or formats for utilizing antibody structural and functional features in alternative presentation. For example, an antibody agent utilized in accordance with the present invention is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (SMIPs™); single chain or Tandem diabodies (TandAb®); VHHs; Anticalins®; Nanobodies® minibodies; BiTE®s; ankyrin repeat proteins or DARPINS®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

An antibody or antibody agent used in performing the methods of the instant disclosure can be single chained or double chained. In some embodiments, the antibody or antigen binding molecule is single chained. In certain embodiments, the antigen binding molecule is selected from the group consisting of an scFv, a Fab, a Fab′, a Fv, a F(ab′) 2, a dAb, and any combination thereof.

Antibodies and antibody agents include antibody fragments. An antibody fragment comprises a portion of an intact antibody, such as the antigen binding or variable region of the intact antibody. Antibody fragments include, but are not limited to, Fab, Fab′, Fab′-SH, F(ab′)₂, Fv, diabody, linear antibodies, multispecific formed from antibody fragments antibodies and scFv fragments, and other fragments. Antibodies also include, but are not limited to, polyclonal monoclonal, chimeric dAb (domain antibody), single chain, Fab, Fa, F(ab′)₂ fragments, and scFvs. An antibody can be a whole antibody, or immunoglobulin, or an antibody fragment. Antibody fragments can be made by various techniques, including but not limited to proteolytic digestion of an intact antibody as well as production by recombinant host cells (e.g., E. coli, Chinese Hamster Ovary (CHO) cells, or phage), as known in the art.

In some embodiments, an antibody or antibody agent can be a chimeric antibody (see, e.g., U.S. Pat. No. 4,816,567; and Morrison et al., Proc. Natl. Acad. Sci. USA, 81:6851-6855 (1984)). A chimeric antibody can be an antibody in which a portion of the heavy and/or light chain is derived from a particular source or species, while the remainder of the heavy and/or light chain is derived from a different source or species. In one example, a chimeric antibody can comprise a non-human variable region (e.g., a variable region derived from a mouse, rat, hamster, rabbit, or non-human primate, such as a monkey) and a human constant region. In a further example, a chimeric antibody can be a “class switched” antibody in which the class or subclass has been changed from that of the parent antibody. Chimeric antibodies include antigen-binding fragments thereof.

In some embodiments, a chimeric antibody can be a humanized antibody (See, e.g., Almagro and Fransson, Front. Biosci., 13:1619-1633 (2008); Riechmann et al., Nature, 332:323-329 (1988); Queen et al., Proc. Natl Acad. Sci. USA 86:10029-10033 (1989); U.S. Pat. Nos. 5,821,337, 7,527,791, 6,982,321, and 7,087,409; Kashmiri et al., Methods 36:25-34 (2005); Padlan, Mol. Immunol, 28:489-498 (1991); Dall'Acqua et al., Methods, 36:43-60 (2005); Osbourn et al., Methods, 36:61-68 (2005); and Klimka et al., Br. J. Cancer, 83:252-260 (2000)). A humanized antibody is a chimeric antibody comprising amino acid residues from non-human hypervariable regions and amino acid residues from human FRs. In certain embodiments, a humanized antibody will comprise substantially all of at least one, and typically two, variable domains, in which all or substantially all of the hypervariable regions (e.g., CDRs) correspond to those of a non-human antibody, and all or substantially all of the Framework Regions (FRs) correspond to those of a human antibody. A humanized antibody optionally can comprise at least a portion of an antibody constant region derived from a human antibody.

In some embodiments, an antibody or antibody agent provided herein is a human antibody. Human antibodies can be produced using various techniques known in the art (See, e.g., van Dijk and van de Winkel, Curr. Opin. Pharmacol, 5:368-74 (2001); and Lonberg, Curr. Opin. Immunol, 20:450-459 (2008)). A human antibody can be one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies may be prepared using methods well known in the art.

### Chimeric Antigen Receptors

As used herein, chimeric antigen receptors (CARs) are proteins that specifically recognize target antigens (e.g., target antigens on cancer cells). When bound to the target antigen, the CAR can activate the immune cell to attack and destroy the cell bearing that antigen (e.g., the cancer cell). CARs can also incorporate costimulatory or signaling domains to increase their potency. See Krause et al., J. Exp. Med., Volume 188, No. 4, 1998 (619-626); Finney et al., Journal of Immunology, 1998, 161:2791-2797, Song et al., Blood 119:696-706 (2012); Kalos et al., Sci. Transl. Med. 3:95 (2011); Porter et al., N. Engl. J. Med. 365:725-33 (2011), and Gross et al., Annu. Rev. Pharmacol. Toxicol. 56:59-83 (2016); U.S. Pat. Nos. 7,741,465, and 6,319,494.

Chimeric antigen receptors described herein comprise an extracellular domain, a transmembrane domain, and an intracellular domain, wherein the extracellular domain comprises an antigen binding domain that specifically binds to the target.

In some embodiments, antigen-specific CARs further comprise a safety switches and/or one or more monoclonal antibody specific-epitope.

### Antigen Binding Domains

As discussed above, CARs described herein comprise an antigen binding domain. An “antigen binding domain” as used herein means any polypeptide that binds a specified target antigen. In some embodiments, the antigen binding domain binds to an antigen on a tumor cell. In some embodiments, the antigen binding domain binds to an antigen on a cell involved in a hyperproliferative disease.

In some embodiments, the antigen binding domain comprises a variable heavy chain, variable light chain, and/or one or more CDRs described herein. In some embodiments, the antigen binding domain is a single chain variable fragment (scFv), comprising light chain CDRs CDR1, CDR2 and CDR3, and heavy chain CDRs CDR1, CDR2 and CDR3.

An antigen binding domain is said to be “selective” when it binds to one target more tightly or with higher affinity than it binds to a second target.

The antigen binding domain of the CAR selectively targets a cancer antigen. In some embodiments, the cancer antigen is selected from EGFRVIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, CD52 or CD34. In some embodiments, the CAR comprises an antigen binding domain that targets EGFRVIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, CD52 or CD34.

In some embodiments, the cancer antigen is selected from the group consisting of carbonic anhydrase IX (CAIX), carcinoembryonic antigen (CEA), CDS, CD7, CDIO, CD19, CD20, CD22, CD30, CD33, CD34, CD38, CD41, CD44, CD49f, CD56, CD74, CD123, CD133, CD138, an antigen of a cytomegalovirus (CMV) infected cell (e.g., a cell surface antigen), epithelial glycoprotein (EGP 2), epithelial glycoprotein-40 (EGP-40), epithelial cell adhesion molecule (EpCAM), receptor tyrosine-protein kinases erb-B2,3,4, folate-binding protein (FBP), fetal acetylcholine receptor (AChR), folate receptors, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Ra2), κ-light chain, kinase insert domain receptor (KDR), Lewis A (CA19.9), LI cell adhesion molecule (LICAM), melanoma antigen family A, 1 (MAGE-AI), Mucin 16 (Muc-16), Mucin 1 (Muc-1), Mesothelin (MSLN), NKG2D ligands, cancer-testis antigen NY-ESO-1, oncofetal antigen (h5T4), prostate stem cell antigen (PSCA), prostate-specific membrane antigen (PSMA), tumor-associated glycoprotein 72 (TAG-72), vascular endothelial growth factor R2 (VEGF-R2), and Wilms tumor protein (WT-1).

Variants of the antigen binding domains (e.g., variants of the CDRs, VH and/or VL) are also within the scope of the disclosure, e.g., variable light and/or variable heavy chains that each have at least 70-80%, 80-85%, 85-90%, 90-95%, 95-97%, 97-99%, or above 99% identity to the amino acid sequences of antigen binding domain sequences. In some instances, such molecules include at least one heavy chain and one light chain, whereas in other instances the variant forms contain two variable light chains and two variable heavy chains (or subparts thereof). A skilled artisan will be able to determine suitable variants of the antigen binding domains as set forth herein using well-known techniques. In certain embodiments, one skilled in the art can identify suitable areas of the molecule that can be changed without destroying activity by targeting regions not believed to be important for activity.

In some embodiments, the polypeptide structure of the antigen binding domains is based on antibodies, including, but not limited to, monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the antigen binding domain comprises or consists of avimers.

In some embodiments, an antigen binding domain is a scFv. In some embodiments, an antigen-selective CAR comprises a leader or signal peptide.

In other embodiments, the disclosure relates to isolated polynucleotides encoding any one of the antigen binding domains described herein. In some embodiments, the disclosure relates to isolated polynucleotides encoding a CAR. Also provided herein are vectors comprising the polynucleotides, and methods of making same.

In other embodiments, the disclosure relates to isolated polynucleotides encoding any one of the antigen binding domains described herein. In some embodiments, the disclosure relates to isolated polynucleotides encoding a CAR. Also provided herein are vectors comprising the polynucleotides, and methods of making same.

In some embodiments, a CAR-immune cell (e.g., CAR-T cell) which can form a component of a population of cells generated by practicing the methods of the instant disclosure comprises a polynucleotide encoding a safety switch polypeptide, such as for example RQR8. See, e.g., WO2013153391A, which is hereby incorporated by reference in its entirety. In a CAR-immune cell (e.g., a CAR-T cell) comprising the polynucleotide, the safety switch polypeptide can be expressed at the surface of a CAR-immune cell (e.g., CAR-T cell).

### Hinge Domains

The extracellular domain of the CARs of the disclosure can comprise a “hinge” domain (or hinge region). The term generally refers to any polypeptide that functions to link the transmembrane domain in a CAR to the extracellular antigen binding domain in a CAR. In particular, hinge domains can be used to provide more flexibility and accessibility for the extracellular antigen binding domain.

A hinge domain can comprise up to 300 amino acids—in some embodiments 10 to 100 amino acids or in some embodiments 25 to 50 amino acids. The hinge domain can be derived from all or part of naturally occurring molecules, such as from all or part of the extracellular region of CD8, CD4, CD28, 4-1BB, or IgG (in particular, the hinge region of an IgG; it will be appreciated that the hinge region can contain some or all of a member of the immunoglobulin family such as IgG1, IgG2, IgG3, IgG4, IgA, IgD, IgE, IgM, or fragment thereof), or from all or part of an antibody heavy-chain constant region. Alternatively, the hinge domain can be a synthetic sequence that corresponds to a naturally occurring hinge sequence, or can be an entirely synthetic hinge sequence. In some embodiments said hinge domain is a part of human CD8a chain (e.g., NP_001139345.1). In other embodiments, said hinge and transmembrane domains comprise a part of human CD8α chain. In some embodiments, the hinge domain of CARs described herein comprises a subsequence of CD8a, an IgG1, IgG4, PD-1 or an FcγRIIIa, in particular the hinge region of any of an CD8a, an IgG1, IgG4, PD-1 or an FcγRIIIa. In some embodiments, the hinge domain comprises a human CD8α hinge, a human IgG1 hinge, a human IgG4, a human PD-1 or a human FcγRIIIa hinge. In some embodiments the CARs disclosed herein comprise a scFv, CD8α human hinge and transmembrane domains, the CD3ζ signaling domain, and 4-1BB signaling domain.

### Transmembrane Domains

The CARs of the disclosure are designed with a transmembrane domain that is fused to the extracellular domain of the CAR. It can similarly be fused to the intracellular domain of the CAR. In some instances, the transmembrane domain can be selected or modified by amino acid substitution to avoid binding of such domains to the transmembrane domains of the same or different surface membrane proteins to minimize interactions with other members of the receptor complex. In some embodiments, short linkers can form linkages between any or some of the extracellular, transmembrane, and intracellular domains of the CAR.

Suitable transmembrane domains for a CAR disclosed herein have the ability to (a) be expressed at the surface an immune cell such as, for example without limitation, a lymphocyte cell, such as a T helper (Tₕ) cell, cytotoxic T (T_{c}) cell, T regulatory (T_{reg}) cell, or Natural killer (NK) cells, and/or (b) interact with the extracellular antigen binding domain and intracellular signaling domain for directing the cellular response of an immune cell against a target cell.

The transmembrane domain can be derived either from a natural or from a synthetic source. Where the source is natural, the domain can be derived from any membrane-bound or transmembrane protein.

Transmembrane regions of particular use in this disclosure can be derived from (comprise, or correspond to) CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptors, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

As non-limiting examples, the transmembrane region can be derived from, or be a portion of a T cell receptor such as α, β, γ or δ, polypeptide constituting CD3 complex, IL-2 receptor p55 (α chain), p75 (β chain) or γ chain, subunit chain of Fc receptors, in particular Fcγ receptor III or CD proteins. Alternatively, the transmembrane domain can be synthetic and can comprise predominantly hydrophobic residues such as leucine and valine. In some embodiments said transmembrane domain is derived from the human CD8α chain (e.g., NP_001139345.1).

In some embodiments, the transmembrane domain in the CAR of the disclosure is a CD8α transmembrane domain.

In some embodiments, the transmembrane domain in the CAR of the disclosure is a CD28 transmembrane domain.

### Intracellular Domains

The intracellular (cytoplasmic) domain of the CARs of the disclosure can provide activation of at least one of the normal effector functions of the immune cell comprising the CAR. Effector function of a T cell, for example, can refer to cytolytic activity or helper activity, including the secretion of cytokines.

In some embodiments, an activating intracellular signaling domain for use in a CAR can be the cytoplasmic sequences of, for example without limitation, the T cell receptor and co-receptors that act in concert to initiate signal transduction following antigen receptor engagement, as well as any derivative or variant of these sequences and any synthetic sequence that has the same functional capability.

It will be appreciated that suitable (e.g., activating) intracellular domains include, but are not limited to signaling domains derived from (or corresponding to) CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptors, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

The intracellular domains of the CARs of the disclosure can incorporate, in addition to the activating domains described above, co-stimulatory signaling domains (interchangeably referred to herein as costimulatory molecules) to increase their potency. Costimulatory domains can provide a signal in addition to the primary signal provided by an activating molecule as described herein.

It will be appreciated that suitable costimulatory domains within the scope of the disclosure can be derived from (or correspond to) for example, CD28, OX40, 4-1BB/CD137, CD2, CD3 (alpha, beta, delta, epsilon, gamma, zeta), CD4, CD5, CD7, CD9, CD16, CD22, CD27, CD30, CD 33, CD37, CD40, CD 45, CD64, CD80, CD86, CD134, CD137, CD154, PD-1, ICOS, lymphocyte function-associated antigen-1 (LFA-1 (CD1 1a/CD18), CD247, CD276 (B7-H3), LIGHT (tumor necrosis factor superfamily member 14; TNFSF14), NKG2C, Ig alpha (CD79a), DAP-10, Fc gamma receptor, MHC class I molecule, TNFR, integrin, signaling lymphocytic activation molecule, BTLA, Toll ligand receptors, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD1-1d, ITGAE, CD103, ITGAL, CD1-1a, LFA-1, ITGAM, CD1-1b, ITGAX, CD1-1c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD83 ligand, or fragments or combinations thereof. It will be appreciated that additional costimulatory molecules, or fragments thereof, not listed above are within the scope of the disclosure.

In some embodiments, the intracellular/cytoplasmic domain of the CAR can be designed to comprise the 4-1BB/CD137 domain by itself or combined with any other desired intracellular domain(s) useful in the context of the CAR of the disclosure. The complete native amino acid sequence of 4-1BB/CD137 is described in NCBI Reference Sequence: NP_001552.2. The complete native 4-1BB/CD137 nucleic acid sequence is described in NCBI Reference Sequence: NM_001561.5.

In some embodiments, the intracellular/cytoplasmic domain of the CAR can be designed to comprise the CD28 domain by itself or combined with any other desired intracellular domain(s) useful in the context of the CAR of the disclosure. The complete native amino acid sequence of CD28 is described in NCBI Reference Sequence: NP_006130.1. The complete native CD28 nucleic acid sequence is described in NCBI Reference Sequence: NM_006139.1.

In some embodiments, the intracellular/cytoplasmic domain of the CAR can be designed to comprise the CD3 zeta domain by itself or combined with any other desired intracellular domain(s) useful in the context of the CAR of the disclosure.

For example, the intracellular domain of the CAR can comprise a CD3 zeta chain portion and a portion of a costimulatory signaling molecule. The intracellular signaling sequences within the intracellular signaling portion of the CAR of the disclosure can be linked to each other in a random or specified order. In some embodiments, the intracellular domain is designed to comprise the activating domain of CD3 zeta and a signaling domain of CD28. In some embodiments, the intracellular domain is designed to comprise the activating domain of CD3 zeta and a signaling domain of 4-1BB.

In some embodiments the intracellular signaling domain of the CAR of the disclosure comprises a domain of a co-stimulatory molecule. In some embodiments, the intracellular signaling domain of a CAR of the disclosure comprises a part of co-stimulatory molecule selected from the group consisting of fragment of 4-1BB (GenBank: AAA53133.) and CD28 (NP_006130.1).

### Genetic Modifications of CAR T Cells

Also provided herein are engineered immune cells and populations of engineered immune cells expressing CAR (e.g., CAR-T cells or CAR+ cells), which are depleted of cells expressing endogenous TCR.

In some embodiments, an engineered immune cell comprises a CAR T cell, each CAR T cell comprising an extracellular antigen-binding domain and has reduced or eliminated expression of endogenous TCR. In some embodiments, a population of engineered immune cells comprises a population of CAR T cells, each CAR T cell comprising two or more different extracellular antigen-binding domain and has reduced or eliminated expression of endogenous TCR. In some embodiments, an immune cell comprises a population of CARs, each CAR T cell comprising the same extracellular antigen-binding domains and has reduced or eliminated expression of endogenous TCR.

In some embodiments, an engineered immune cell according to the present disclosure comprises one disrupted or inactivated gene selected from the group consisting of CD52, DLL3, GR, PD-1, CTLA-4, LAG3, TIM3, BTLA, BY55, TIGIT, B7H5, LAIR1, SIGLEC10, 2B4, HLA, TCRα and TCRβ and/or expresses a CAR, a multi-chain CAR and/or a pTα transgene. In some embodiments, an isolated cell comprises polynucleotides encoding polypeptides comprising a multi-chain CAR. In some embodiments, the isolated cell according to the present disclosure comprises two disrupted or inactivated genes selected from the group consisting of: CD52 and GR, CD52 and TCRα, CDR52 and TCRB, DLL3 and CD52, DLL3 and TCRα, DLL3 and TCRβ, GR and TCRα, GR and TCRβ, TCRα and TCRβ, PD-1 and TCRα, PD-1 and TCRβ, CTLA-4 and TCRα, CTLA-4 and TCRβ, LAG3 and TCRα, LAG3 and TCRβ, TIM3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ and/or expresses a CAR, including a multi-chain CAR, and/or a pTα transgene. In some embodiments the method comprises disrupting or inactivating one or more genes by introducing into the cells an endonuclease capable of selectively inactivating a gene by selective DNA cleavage. In some embodiments the endonuclease can be, for example, a zinc finger nuclease (ZFN), a modified-homing endonuclease (e.g., meganuclease), megaTAL nuclease, transcription activator-like effector nuclease (TALE-nuclease, or TALEN®), or CRISPR (e.g., Cas9 or Cas12) endonuclease.

In some embodiments, TCR is rendered not functional in the cells according to the disclosure by disrupting or inactivating TCRα gene and/or TCRβ gene(s). In some embodiments, a method to obtain modified cells derived from an individual is provided, wherein the cells can proliferate independently of the major histocompatibility complex (MHC) signaling pathway. Modified cells, which can proliferate independently of the MHC signaling pathway, susceptible to be obtained by this method are encompassed in the scope of the present disclosure. Modified cells disclosed herein can be used for treating patients in need thereof against Host versus Graft (HvG) rejection and Graft versus Host Disease (GvHD); therefore in the scope of the present disclosure is a method of treating patients in need thereof against Host versus Graft (HvG) rejection and Graft versus Host Disease (GvHD) comprising treating said patient by administering to said patient an effective amount of modified cells comprising disrupted or inactivated TCRα and/or TCRβ genes.

The present disclosure provides methods of determining the purity of a population of engineered immune cells lacking or having reduced endogenous TCR expression. In some embodiments, the engineered immune cells comprise less than 5.0%, less than 4.0%, less than 3.0% TCR+ cells, less than 2.0% TCR+ cells, less than 1.0% TCR+ cells, less than 0.9% TCR+ cells, less than 0.8% TCR+ cells, less than 0.7% TCR+ cells, less than 0.6% TCR+ cells, less than 0.5% TCR+ cells, less than 0.4% TCR+ cells, less than 0.3% TCR+ cells, less than 0.2% TCR+ cells, or less than 0.1% TCR+ cells. Such a population can be a product of the disclosed methods.

In some embodiments, an engineered immune cell according to the present disclosure can comprise one or more disrupted or inactivated genes. In some embodiments, a gene for a target antigen (e.g., EGFRvIII, Flt3, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, or CD34, CD70) can be knocked out to introduce a CAR targeting the same antigen (e.g., a EGFRvIII, Flt3, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, or CD34, CD70 CAR) to avoid induced CAR activation. As described herein, in some embodiments, an engineered immune cell according to the present disclosure comprises one disrupted or inactivated gene selected from the group consisting of MHC1 (β2M), MHC2 (CIITA), EGFRvIII, Flt3, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, or CD34, CD70, TCRα and TCRβ and/or expresses a CAR or a multi-chain CAR. In some embodiments, a cell comprises a multi-chain CAR. In some embodiments, the isolated cell comprises two disrupted or inactivated genes selected from the group consisting of: CD52 and TCRα, CDR52 and TCRβ, PD-1 and TCRα, PD-1 and TCRβ, MHC-1 and TCRα, MHC-1 and TCRβ, MHC2 and TCRα, MHC2 and TCRβ and/or expresses a CAR or a multi-chain CAR.

The engineered immune cells can be allogeneic or autologous.

In some embodiments, an engineered immune cell or population of engineered immune cells comprises a T cell (e.g., inflammatory T-lymphocyte, cytotoxic T-lymphocyte, regulatory T-lymphocyte, helper T-lymphocyte, tumor infiltrating lymphocyte (TIL)), NK cell, NK-T-cell, TCR-expressing cell, dendritic cell, killer dendritic cell, a mast cell, or a B-cell, and expresses a CAR. In some embodiments, the T cell can be derived from the group consisting of CD4+T lymphocytes, CD8+T lymphocytes or population comprising a combination of CD4+ and CD8+ T cells.

In some embodiments, an engineered immune cell or population of engineered immune cells that are generated using the disclosed methods can be derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells.

In some embodiments, an engineered immune cell or a population of immune cells that are generated using the disclosed methods is obtained or prepared from peripheral blood. In some embodiments, an engineered immune cell is obtained or prepared from peripheral blood mononuclear cells (PBMCs). In some embodiments, an engineered immune cell is obtained or prepared from bone marrow. In some embodiments, an engineered immune cell is obtained or prepared from umbilical cord blood. In some embodiments, the cell is a human cell. In some embodiments, the cell is transfected or transduced by the nucleic acid vector using a method selected from the group consisting of electroporation, sonoporation, biolistics (e.g., Gene Gun), lipid transfection, polymer transfection, nanoparticles, viral transfection (e.g., retrovirus, lentivirus, AAV) or polyplexes.

In some embodiments, the engineered immune cells expressing at their cell surface membrane an antigen-specific CAR comprise a percentage of stem cell memory and central memory cells greater than 10%, 20%, 30%, 40%, 50%, 60%, 70%, 80%, 90%, or 100%.

In some embodiments, engineered immune cells expressing at their cell surface membrane an antigen-specific CAR comprise a percentage of stem cell memory and central memory cells of about 10% to about 60%, about 10% to about 50%, about 10% to about 40%, about 15% to about 50%, about 15% to about 40%, about 20% to about 60%, or about 20% to about 70%.

In some embodiments, engineered immune cells expressing at their cell surface membrane an antigen-specific CAR enriched in T_{CM} and/or T_{SCM} cells such that the engineered immune cells comprise at least about 60%, 65%, 70%, 75%, or 80% combined T_{CM} and T_{SCM} cells. In some embodiments, engineered immune cells expressing at their cell surface membrane an antigen-specific CAR are enriched in T_{CM} and/or T_{SCM} cells such that the engineered immune cells comprise at least about 70% combined T_{CM} and T_{SCM} cells. In some embodiments, engineered immune cells expressing at their cell surface membrane an antigen-specific CAR e enriched in T_{CM} and/or T_{SCM} cells such that the engineered immune cells comprise at least about 75% combined T_{CM} and/or T_{SCM} cells.

In some embodiments, an engineered immune cell is an inflammatory T-lymphocyte that expresses a CAR. In some embodiments, an engineered immune cell is a cytotoxic T-lymphocyte that expresses a CAR. In some embodiments, an engineered immune cell is a regulatory T-lymphocyte that expresses a CAR. In some embodiments, an engineered immune cell is a helper T-lymphocyte that expresses a CAR.

In some embodiments, the immune cells are engineered to be resistant to one or more chemotherapy drugs. The chemotherapy drug can be, for example, a purine nucleotide analogue (PNA), thus making the immune cell suitable for cancer treatment combining adoptive immunotherapy and chemotherapy. Exemplary PNAs include, for example, clofarabine, fludarabine, cyclophosphamide, and cytarabine, alone or in combination. PNAs are metabolized by deoxycytidine kinase (dCK) into mono-, di-, and tri-phosphate PNA. Their tri-phosphate forms compete with ATP for DNA synthesis, act as pro-apoptotic agents, and are potent inhibitors of ribonucleotide reductase (RNR), which is involved in trinucleotide production.

In some embodiments, isolated cells or cell lines of the disclosure can comprise a pTα or a functional variant thereof. In some embodiments, an isolated cell or cell line can be further genetically modified by disrupting or inactivating the TCRα gene.

The disclosure also provides engineered immune cells comprising any of the CAR polynucleotides described herein. In some embodiments, a CAR can be introduced into an immune cell as a transgene via a plasmid vector. In some embodiments, the plasmid vector can also contain, for example, a selection marker which provides for identification and/or selection of cells which received the vector.

CAR polypeptides can be synthesized in situ in the cell after introduction of polynucleotides encoding the CAR polypeptides into the cell. Alternatively, CAR polypeptides can be produced outside of cells, and then introduced into cells. Methods for introducing a polynucleotide construct into cells are known in the art. In some embodiments, stable transformation methods (e.g., using a lentiviral vector) can be used to integrate the polynucleotide construct into the genome of the cell. In other embodiments, transient transformation methods can be used to transiently express the polynucleotide construct, and the polynucleotide construct not integrated into the genome of the cell. In other embodiments, virus-mediated methods can be used. The polynucleotides can be introduced into a cell by any suitable means such as for example, recombinant viral vectors (e.g., retroviruses, adenoviruses), liposomes, and the like. Transient transformation methods include, for example without limitation, microinjection, electroporation or particle bombardment. Polynucleotides can be included in vectors, such as for example plasmid vectors or viral vectors.

In some embodiments, isolated nucleic acids are provided comprising a promoter operably linked to a first polynucleotide encoding an antigen binding domain, at least one costimulatory molecule, and an activating domain. In some embodiments, the nucleic acid construct is contained within a viral vector. In some embodiments, the viral vector is selected from the group consisting of retroviral vectors, murine leukemia virus vectors, SFG vectors, adenoviral vectors, lentiviral vectors, adeno-associated virus (AAV) vectors, Herpes virus vectors, and vaccinia virus vectors. In some embodiments, the nucleic acid is contained within a plasmid.

In some embodiments, the isolated nucleic construct is contained within a viral vector and is introduced into the genome of an engineered immune cell by random integration, e.g., lentiviral- or retroviral-mediated random integration. In some embodiments, the isolated nucleic acid construct is contained in a viral vector or a non-viral vector and is introduced into the genome of an engineered immune cell by site-specific integration, e.g., adenovirus-mediated site-specific integration.

### Manufacture of Engineered Immune Cells

A variety of known techniques can be utilized in making the polynucleotides, polypeptides, vectors, antigen binding domains, immune cells, compositions, and the like according to the disclosure.

Prior to the in vitro manipulation or genetic modification of the immune cells described herein, the cells can be obtained from a subject. Cells expressing a CAR can be derived from an allogeneic or autologous source and can be depleted of endogenous TCR as described herein.

### Source Material

In some embodiments, the immune cells comprise T cells. T cells can be obtained from a number of sources, including peripheral blood mononuclear cells (PBMCs), bone marrow, lymph nodes tissue, cord blood, thymus tissue, tissue from a site of infection, ascites, pleural effusion, spleen tissue, and tumors. In some embodiments, T cells can be obtained from a volume of blood collected from the subject using any number of techniques known to the skilled person, such as FICOLL™ separation.

Cells can be obtained from the circulating blood of an individual by apheresis. The apheresis product typically contains lymphocytes, including T cells, monocytes, granulocytes, B cells, other nucleated white blood cells, red blood cells, and platelets. In some embodiments, the cells collected by apheresis can be washed to remove the plasma fraction, and then placed in an appropriate buffer or media for subsequent processing.

In some embodiments, T cells are isolated from PBMCs by lysing the red blood cells and depleting the monocytes, for example, using centrifugation through a PERCOLL™ gradient. A specific subpopulation of T cells, (e.g., CD28+, CD4+, CD45RA−, and CD45RO+ T cells or CD28+, CD4+, CDS+, CD45RA−, CD45RO+, and CD62L+ T cells) can be further isolated by positive or negative selection techniques known in the art. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. Flow cytometry and cell sorting can also be used to isolate cell populations of interest for use in the present disclosure.

PBMCs can be used directly for genetic modification with the immune cells (such as CARs or TCRs) using methods as described herein. In certain embodiments, after isolating the PBMCs, T lymphocytes can be further isolated and both cytotoxic and helper T lymphocytes can be sorted into naive, memory, and effector T cell subpopulations either before or after genetic modification and/or expansion. In some embodiments, CD8+ cells are further sorted into naive, stem cell memory, central memory, and effector cells by identifying cell surface antigens that are associated with each of these types of CD8+ cells. In some embodiments, the expression of phenotypic markers of central memory T cells include CD27, CD45RA, CD45RO, CD62L, CCR7, CD28, CD3, and CD127 and are negative for granzyme B. In some embodiments, stem cell memory T cells are CD45RO−, CD62L+, CD8+ T cells. In some embodiments, central memory T cells are CD45RO+, CD62L+, CD8+ T cells. In some embodiments, effector T cells are negative for CD62L, CCR7, CD28, and CD127, and positive for granzyme B and perforin. In some embodiments, CD4+ T cells are further sorted into subpopulations. For example, CD4+T helper cells can be sorted into naive, central memory, and effector cells by identifying cell populations that have cell surface antigens.

### Stem Cell Derived Immune Cells

In some embodiments, the immune cells can be derived from embryonic stem (ES) or induced pluripotent stem (iPS) cells. Suitable HSCs, mesenchymal, iPS cells and other types of stem cells can be cultivated immortal cell lines or isolated directly from a patient. Various methods for isolating, developing, and/or cultivating stem cells are known in the art and can be used to practice the present disclosure.

In some embodiments, the immune cell is an induced pluripotent stem cell (iPSC) derived from a reprogrammed T-cell. In some embodiments, the source material can be an induced pluripotent stem cell (iPSC) derived from a T cell or a non-T cell. In some embodiments, the immune cell is an iPSC-derived T cell. In some embodiments, the immune cell is an iPSC-derived NK cells. The source material can be an embryonic stem cell. The source material can be a B cell, or any other cell from peripheral blood mononuclear cell isolates, hematopoietic progenitor, hematopoietic stem cell, mesenchymal stem cell, adipose stem cell, or any other somatic cell type.

### Genetic Modification of Isolated Cells

The immune cells, such as T cells, can be genetically modified following isolation using known methods, or the immune cells can be activated and expanded (or differentiated in the case of progenitors) in vitro prior to being genetically modified. In some embodiments, the isolated immune cells are genetically modified to reduce or eliminate expression of endogenous TCRα and/or CD52. In some embodiments, the cells are genetically modified using gene editing technology (e.g., CRISPR/Cas9, CRISPR/Cas12a, a zinc finger nuclease (ZFN), a TALEN, a MegaTAL, a meganuclease) to reduce or eliminate expression of endogenous proteins (e.g., TCRα and/or CD52). In another embodiment, the immune cells, such as T cells, are genetically modified with the chimeric antigen receptors described herein (e.g., transduced with a viral vector comprising one or more nucleotide sequences encoding a CAR) and then are activated and/or expanded in vitro.

Certain methods for making the constructs and engineered immune cells of the disclosure are described in PCT application PCT/US15/14520, the contents of which are hereby incorporated by reference in their entirety.

It will be appreciated that PBMCs can further include other cytotoxic lymphocytes such as NK cells or NKT cells. An expression vector carrying the coding sequence of a chimeric receptor as disclosed herein can be introduced into a population of human donor T cells, NK cells or NKT cells. Successfully transduced T cells that carry the expression vector can be sorted using flow cytometry to isolate CD3 positive T cells and then further propagated to increase the number of these CAR expressing T cells in addition to cell activation using anti-CD3 antibodies and IL-2 or other methods known in the art as described elsewhere herein. Standard procedures are used for cryopreservation of T cells expressing the CAR for storage and/or preparation for use in a human subject. In one embodiment, the in vitro transduction, culture and/or expansion of T cells are performed in the absence of non-human animal derived products such as fetal calf serum and fetal bovine serum.

For cloning of polynucleotides, the vector can be introduced into a host cell (an isolated host cell) to allow replication of the vector itself and thereby amplify the copies of the polynucleotide contained therein. The cloning vectors can contain sequence components generally include, without limitation, an origin of replication, promoter sequences, transcription initiation sequences, enhancer sequences, and selectable markers. These elements can be selected as appropriate by a person of ordinary skill in the art. For example, the origin of replication can be selected to promote autonomous replication of the vector in the host cell.

In some embodiments, the present disclosure provides isolated host cells containing the vector provided herein. The host cells containing the vector can be useful in expression or cloning of the polynucleotide contained in the vector. Suitable host cells can include, without limitation, prokaryotic cells, fungal cells, yeast cells, or higher eukaryotic cells such as mammalian cells, particularly human cells.

The vector can be introduced to the host cell using any suitable methods known in the art, including, without limitation, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transfection and transformation of cells for expression of a vector of interest are well known in the art. In a further embodiment, a mixture of different expression vectors can be used in genetically modifying a donor population of immune effector cells wherein each vector encodes a different CAR as disclosed herein. The resulting transduced immune effector cells form a mixed population of engineered cells, with a proportion of the engineered cells expressing more than one different CARS.

In one embodiment, the disclosure provides a method of storing genetically engineered cells expressing CARs or TCRs. This involves cryopreserving the immune cells such that the cells remain viable upon thawing. A fraction of the immune cells expressing the CARs can be cryopreserved by methods known in the art to provide a permanent source of such cells for the future treatment of patients afflicted with a malignancy. When needed, the cryopreserved transformed immune cells can be thawed, grown and expanded for more such cells.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion media can be any isotonic medium formulation, typically normal saline, Normosol™ R (Abbott) or Plasma-Lyte™ A (Baxter), but also 5% dextrose in water or Ringer's lactate can be utilized. The infusion medium can be supplemented with human serum albumin.

### Allogeneic CAR T Cells

The process for manufacturing allogeneic CAR T therapy involves harvesting healthy, selected, screened and tested T cells from healthy donors. Next, the T cells are engineered to express CARs, which recognize certain cell surface proteins that are expressed in hematologic or solid tumors. Allogeneic T cells are gene edited to reduce the risk of graft versus host disease (GvHD) and to prevent allogeneic rejection. A T cell receptor gene (e.g., TCRα, TCRβ) is knocked out to avoid GvHD. The TCRα loci is located on chromosome 14 where inversions have been shown to occur, such as at 14q11 as described herein. The present disclosure provides methods particularly advantageous for the analysis of allogeneic immune cells or immune cell populations comprising engineered cells having any gene modifications on chromosome 14, such as gene-edited targets on chromosome 14. Specifically, the methods allow for the determination of the presence or absence of a chromosome 14 inversion at the TCR A/D, e.g., at 14q11.2, in cells that have been engineered, e.g., gene edited, to inactivate or change the expression of one or more genes on chromosome 14 on a genomic level. In one embodiment, the cells have been engineered to knockout expression of the TCRα gene on chromosome 14, such as by knocking out at least a portion of TCRα. In other embodiments, the chromosome 14 inversion comprises a centromeric and a telomeric inversion site. In one other embodiment, the centromeric inversion site is at the T cell receptor joining gene TRAJ7. In one other embodiment, the telomeric inversion site of the chromosome 14 inversion is at the IGHV3-69-1 pseudogene. In another embodiment, the chromosome 14 inversion is (i) located between TRAJ7 and IGHV3-69-1 and/or (ii) a 14q11 inversion.

The CD52 gene can be knocked out to render the CAR T product resistant to anti-CD52 antibody treatment. Anti-CD52 antibody treatment can therefore be used to suppress the host immune system and allow the CAR T to stay engrafted to achieve full therapeutic impact. The engineered T cells then undergo a purification step and are ultimately cryopreserved in vials for delivery to patients.

### Autologous CAR T Cells

Autologous chimeric antigen receptor (CAR) T cell therapy, involves collecting a patient's own cells (e.g., white blood cells, including T cells) and genetically engineering the T cells to express CARs that recognize target expressed on the cell surface of one or more specific cancer cells and kill cancer cells. The engineered cells are then cryopreserved and subsequently administered to the patient.

### Pharmaceutical Compositions

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion media can be any isotonic medium formulation, typically normal saline, Normosol™ R (Abbott) or Plasma-Lyte™ A (Baxter), but also 5% dextrose in water or Ringer's lactate can be utilized. The infusion medium can be supplemented with human serum albumin.

In embodiments, desired treatment amounts of cells in the composition are generally at least 2 cells (for example, at least 1 CD8+ central or stem cell memory T cell and at least 1 CD4+ helper T cell subset; or two or more CD8+ central or stem cell memory T cell; or two or more CD4+ helper T cell subset) or is more typically greater than 10² cells, and up to and including 10⁶, up to and including 10⁷, 10⁸ or 10⁹ cells and can be more than 10¹⁰ cells. The number of cells will depend upon the desired use for which the composition is intended, and the type of cells included therein. The density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present disclosure, particularly since all the infused cells will be redirected to a particular target antigen, lower numbers of cells, in the range of about 10⁵/kilogram or about 10⁶/kilogram (10⁶-10¹¹ per patient) can be administered. CAR treatments can be administered multiple times at dosages within these ranges. The cells can be autologous, allogeneic, or heterologous to the patient undergoing therapy.

The CAR expressing cell populations of the present disclosure can be administered either alone, or as a pharmaceutical composition in combination with diluents and/or with other components such as IL-2 or other cytokines or cell populations. Pharmaceutical compositions of the present disclosure can comprise a CAR or TCR expressing cell population, such as T cells, as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for intravenous administration.

The pharmaceutical compositions (solutions, suspensions or the like), can include one or more of the following: sterile diluents such as water for injection, saline solution, preferably physiological saline, Ringer's solution, isotonic sodium chloride, fixed oils such as synthetic mono- or diglycerides which can serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampules, disposable syringes or multiple dose vials made of glass or plastic. An injectable pharmaceutical composition is preferably sterile.

### Methods of Treatment

The disclosure comprises methods for treating or preventing a disease (e.g., cancer) in a patient, comprising administering to a patient in need thereof an effective amount of CAR T cells, or engineered immune cells comprising a CAR disclosed herein. In some embodiments, the effective amount of CAR T cells or engineered immune cells have been analyzed for various attributes according to the methods described in the instant disclosure. In some embodiments, the CAR T cell drug product for therapeutic use has been analyzed for various attributes, such as potency or polyfunctionality according to the methods described in the instant disclosure. In some embodiments, the CAR T cells are TCR-CAR T cells, and the CAR T drug product for therapeutic use has been analyzed for various attributes, such as the amount or percentage of remaining TCR+CAR T cells and/or potency or polyfunctionality according to the methods described in the instant disclosure.

Methods are provided for treating diseases or disorders, including cancer. In some embodiments, the disclosure relates to creating a T cell-mediated immune response in a subject, comprising administering an effective amount of the engineered immune cells of the present application to the subject. In some embodiments, the T cell-mediated immune response is directed against a target cell or cells. In some embodiments, the engineered immune cell comprises a chimeric antigen receptor (CAR). In some embodiments, the target cell is a tumor cell. In some aspects, the disclosure comprises a method for treating or preventing a malignancy, said method comprising administering to a subject in need thereof an effective amount of at least one isolated antigen binding domain described herein. In some aspects, the disclosure comprises a method for treating or preventing a malignancy, said method comprising administering to a subject in need thereof an effective amount of at least one immune cell, wherein the immune cell comprises at least one chimeric antigen receptor, T cell receptor, and/or isolated antigen binding domain as described herein. The CAR containing immune cells of the disclosure can be used to treat malignancies involving aberrant expression of biomarkers. In some embodiments, CAR containing immune cells of the disclosure can be used to treat small cell lung cancer, melanoma, low grade gliomas, glioblastoma, medullary thyroid cancer, carcinoids, dispersed neuroendocrine tumors in the pancreas, bladder and prostate, testicular cancer, and lung adenocarcinomas with neuroendocrine features. In exemplary embodiments, the CAR containing immune cells, e.g., CAR-T cells of the disclosure are used to treat small cell lung cancer.

Also provided are methods for reducing the size of a tumor in a subject, comprising administering to the subject an engineered cell of the present disclosure to the subject, wherein the cell comprises a chimeric antigen receptor comprising an antigen binding domain and binds to an antigen on the tumor.

In some embodiments, the subject has a solid tumor, or a blood malignancy such as lymphoma or leukemia. In some embodiments, the engineered cell is delivered to a tumor bed. In some embodiments, the cancer is present in the bone marrow of the subject. In some embodiments, the engineered cells are autologous immune cells, e.g., autologous T cells. In some embodiments, the engineered cells are allogeneic immune cells, e.g., allogeneic T cells. In some embodiments, the engineered cells are heterologous immune cells, e.g., heterologous T cells. In some embodiments, the engineered cells of the present application are transfected or transduced in vivo. In other embodiments, the engineered cells are transfected or transduced ex vivo. As used herein, the term “in vitro cell” refers to any cell which is cultured ex vivo.

A “therapeutically effective amount,” “effective dose,” “effective amount,” or “therapeutically effective dosage” of a therapeutic agent, e.g., engineered CART cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “patient” and “subject” are used interchangeably and include human and non-human animal subjects as well as those with formally diagnosed disorders, those without formally recognized disorders, those receiving medical attention, those at risk of developing the disorders, etc.

The term “treat” and “treatment” includes therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. The term “prevent” does not require the 100% elimination of the possibility of an event. Rather, it denotes that the likelihood of the occurrence of the event has been reduced in the presence of the compound or method.

Desired treatment amounts of cells in the composition is generally at least 2 cells (for example, at least 1 CD8+ central memory T cell and at least 1 CD4+ helper T cell subset) or is more typically greater than 10² cells, and up to 10⁶, up to and including 10⁸ or 10⁹ cells and can be more than 10¹⁰ cells. The number of cells will depend upon the desired use for which the composition is intended, and the type of cells included therein. The density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present disclosure, particularly since all the infused cells will be redirected to a particular target antigen, lower numbers of cells, in the range of 10⁶/kilogram (10⁶-10¹¹ per patient) can be administered. CAR treatments can be administered multiple times at dosages within these ranges. The cells can be autologous, allogeneic, or heterologous to the patient undergoing therapy.

In some embodiments, the therapeutically effective amount of the CAR T cells is about 1×10⁵ cells/kg, about 2×10⁵ cells/kg, about 3×10⁵ cells/kg, about 4×10⁵ cells/kg, about 5×10⁵ cells/kg, about 6×10⁵ cells/kg, about 7×10⁵ cells/kg, about 8×10⁵ cells/kg, about 9×10⁵ cells/kg, 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

In some embodiments, target doses for CAR+/CAR−T+/TCR+ cells range from 1×10⁶-2×10⁸ cells/kg, for example 2×10⁶ cells/kg. It will be appreciated that doses above and below this range can be appropriate for certain subjects, and appropriate dose levels can be determined by the healthcare provider as needed. Additionally, multiple doses of cells can be provided in accordance with the disclosure.

In some aspect, the disclosure comprises a pharmaceutical composition comprising at least one antigen binding domain as described herein and a pharmaceutically acceptable excipient. In some embodiments, the pharmaceutical composition further comprises an additional active agent.

The CAR expressing cell populations of the present disclosure can be administered either alone, or as a pharmaceutical composition in combination with diluents and/or with other components such as IL-2 or other cytokines or cell populations. Pharmaceutical compositions of the present disclosure can comprise a CAR or TCR expressing cell population, such as T cells, as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for intravenous administration.

The pharmaceutical compositions (solutions, suspensions or the like), can include one or more of the following: sterile diluents such as water for injection, saline solution, preferably physiological saline, Ringer's solution, isotonic sodium chloride, fixed oils such as synthetic mono- or diglycerides which can serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampules, disposable syringes or multiple dose vials made of glass or plastic. An injectable pharmaceutical composition is preferably sterile.

In some embodiments, upon administration to a patient, engineered immune cells expressing at their cell surface any one of the antigen-specific CARs described herein can reduce, kill or lyse endogenous antigen-expressing cells of the patient. In one embodiment, a percentage reduction or lysis of antigen-expressing endogenous cells or cells of a cell line expressing an antigen by engineered immune cells expressing any one of an antigen-specific CARs described herein is at least about or greater than 10%, 15%, 20%, 25%, 30%, 35%, 40%, 45%, 50%, 55%, 60%, 65%, 70%, 75%, 80%, 85%, 90%, or 95%. In one embodiment, a percentage reduction or lysis of antigen-expressing endogenous cells or cells of a cell line expressing an antigen by engineered immune cells expressing antigen-specific CARs is about 5% to about 95%, about 10% to about 95%, about 10% to about 90%, about 10% to about 80%, about 10% to about 70%, about 10% to about 60%, about 10% to about 50%, about 10% to about 40%, about 20% to about 90%, about 20% to about 80%, about 20% to about 70%, about 20% to about 60%, about 20% to about 50%, about 25% to about 75%, or about 25% to about 60%. In one embodiment, the endogenous antigen-expressing cells are endogenous antigen-expressing bone marrow cells.

In one embodiment, the percent reduction or lysis of target cells, e.g., a cell line expressing an antigen, by engineered immune cells expressing at their cell surface membrane an antigen-specific CAR of the disclosure can be measured using the assay disclosed herein.

The methods can further comprise administering one or more chemotherapeutic agent. In some embodiments, the chemotherapeutic agent is a lymphodepleting (preconditioning) chemotherapeutic. For example, methods of conditioning a patient in need of a T cell therapy comprising administering to the patient specified beneficial doses of cyclophosphamide (between 200 mg/m²/day and 2000 mg/m²/day, about 100 mg/m²/day and about 2000 mg/m²/day; e.g., about 100 mg/m²/day, about 200 mg/m²/day, about 300 mg/m²/day, about 400 mg/m²/day, about 500 mg/m²/day, about 600 mg/m²/day, about 700 mg/m²/day, about 800 mg/m²/day, about 900 mg/m²/day, about 1000 mg/m²/day, about 1500 mg/m²/day or about 2000 mg/m²/day) and specified doses of fludarabine (between 20 mg/m²/day and 900 mg/m²/day, between about 10 mg/m²/day and about 900 mg/m²/day; e.g., about 10 mg/m²/day, about 20 mg/m²/day, about 30 mg/m²/day, about 40 mg/m²/day, about 40 mg/m²/day, about 50 mg/m²/day, about 60 mg/m²/day, about 70 mg/m²/day, about 80 mg/m²/day, about 90 mg/m²/day, about 100 mg/m²/day, about 500 mg/m²/day or about 900 mg/m²/day). A preferred dose regimen involves treating a patient comprising administering daily to the patient about 300 mg/m²/day of cyclophosphamide and about 30 mg/m²/day of fludarabine for three days prior to administration of a therapeutically effective amount of engineered T cells to the patient.

In some embodiments, lymphodepletion further comprises administration of a CD52 antibody. In some embodiments, the CD52 antibody is alemtuzumab. In some embodiments, the CD52 antibody is administered at a dose of about 13 mg/day IV.

In other embodiments, the antigen binding domain, transduced (or otherwise engineered) cells and the chemotherapeutic agent are administered each in an amount effective to treat the disease or condition in the subject.

In some embodiments, compositions comprising CAR-expressing immune effector cells disclosed herein can be administered in conjunction with any number of chemotherapeutic agents, which can be administered in any order. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′, 2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL™, Bristol-Myers Squibb) and doxetaxel (TAXOTERE®, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RF S2000; difluoromethylomithine (DMFO); retinoic acid derivatives such as Targretin™ (bexarotene), Panretin™, (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4 (5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP, i.e., Cyclophosphamide (Cytoxan®), Doxorubicin (hydroxydoxorubicin), Vincristine (Oncovin®), and Prednisone.

In some embodiments, the chemotherapeutic agent is administered at the same time or within one week after the administration of the engineered cell, polypeptide, or nucleic acid. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the engineered cell, polypeptide, or nucleic acid. In other embodiments, the chemotherapeutic agent is administered at least 1 month before administering the cell, polypeptide, or nucleic acid. In some embodiments, the methods further comprise administering two or more chemotherapeutic agents.

A variety of additional therapeutic agents can be used in conjunction with the compositions described herein. For example, potentially useful additional therapeutic agents include PD-1 inhibitors such as nivolumab (Opdivo®), pembrolizumab (Keytruda®), pembrolizumab, pidilizumab, and atezolizumab (Tcentriq®).

Additional therapeutic agents suitable for use in combination with the disclosure include, but are not limited to, ibrutinib (Imbruvica®), ofatumumab (Arzerra®, rituximab (Rituxan®), bevacizumab (Avastin®), trastuzumab (Herceptin®), trastuzumab emtansine (KADCYLA®, imatinib (Gleevec®), cetuximab (Erbitux®, panitumumab) (Vectibix®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept, adipotide, denileukin diftitox, mTOR inhibitors such as Everolimus and Temsirolimus, hedgehog inhibitors such as sonidegib and vismodegib, CDK inhibitors such as CDK inhibitor (palbociclib).

In some embodiments, the composition comprising CAR-containing immune cells can be administered with a therapeutic regimen to prevent cytokine release syndrome (CRS) or neurotoxicity. The therapeutic regimen to prevent cytokine release syndrome (CRS) or neurotoxicity can include lenzilumab, tocilizumab, atrial natriuretic peptide (ANP), anakinra, iNOS inhibitors (e.g., L-NIL or 1400W). In additional embodiments, the composition comprising CAR-containing immune cells can be administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

In certain embodiments, the compositions described herein are administered in conjunction with a cytokine. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, IL-21 a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

### Kits

In an aspect, the present disclosure provides for a kit for assessing and/or detecting the presence of a genome abnormality or variant, e.g., an inversion, in a population of cells.

In one embodiment, the kit comprises a first primer molecule, a second primer molecule, and a probe molecule. In one embodiment, the first primer molecule comprises CCAGAGACAACGCCAAGAACTCA (SEQ ID NO: 1), the second primer molecule comprises CCCCGCTCCCATGGAAATATGA (SEQ ID NO: 2), and the probe molecule comprises CCACAGCCACCCCCATCCCT (SEQ ID NO: 3). In one embodiment, the first primer molecule, second primer molecule, and probe molecule can be used to detect a first breakpoint of an inversion. In another embodiment, the first and second primer molecule correspond to the first breakpoint. In one embodiment, the first breakpoint is a breakpoint of a telomeric inversion.

In another embodiment, the kit may further comprise or may comprise on its own the following: a third primer molecule, a fourth primer molecule, and a second probe molecule. In one embodiment, the third primer molecule comprises AGCAGCCAAATCCTTCAGTC (SEQ ID NO: 4), the fourth primer molecule comprises GCAGCAAGGTTTTTGTCTGG (SEQ ID NO: 5), and the second probe molecule comprises ACTCACAGCTGGGGGATGCC (SEQ ID NO: 6). In one embodiment, the third primer molecule, fourth primer molecule, and second probe molecule can be used to detect a second breakpoint of an inversion. In another embodiment, the third and fourth primer molecule correspond to the second breakpoint. In one embodiment, the second breakpoint is a breakpoint of a centromeric inversion. The probe molecules may comprise a detectable label or moiety. In one embodiment, the probe molecule comprises one or more probe sequences, which may comprise a detectable moiety such as a fluorophore or a fluorescent moiety.

In an aspect, the present disclosure provides for a kit for assessing and/or detecting the presence of a genome abnormality or variant, e.g., a translocation, in a population of cells. In another embodiment, the kit may comprise a fifth primer molecule, a sixth primer molecule, and a third probe molecule for detection of a first region of the translocation. In one embodiment, the fifth primer molecule comprises AGGCCTGGCCGTGAA (SEQ ID NO: 7), the sixth primer molecule comprises CCCAGCCAGGGCCTTA (SEQ ID NO: 8), and the third probe molecule comprises ACTTGCCAGCCCCACAGAGCCCC (SEQ ID NO: 9). In another embodiment, the kit may comprise a seventh primer molecule, an eighth primer molecule, and a fourth probe molecule for detection of a second region of the translocation. In one embodiment, the seventh primer molecule comprises AGCAGTGCAGCCCCA (SEQ ID NO: 10) the eighth primer molecule comprises CTCCAGGCCACAGCAC (SEQ ID NO: 11), and the fourth probe molecule comprises CCAGGCCCGCTGTGTCCCCAGTT (SEQ ID NO: 12).

The following examples are offered for illustrative purposes only. Various modifications of the invention in addition to those shown and described herein will become apparent to those skilled in the art from the foregoing description.

### EXAMPLES

### Example 1—Spontaneous Post Infusion Chromosome 14 Rearrangement in Allogeneic CAR T Cells

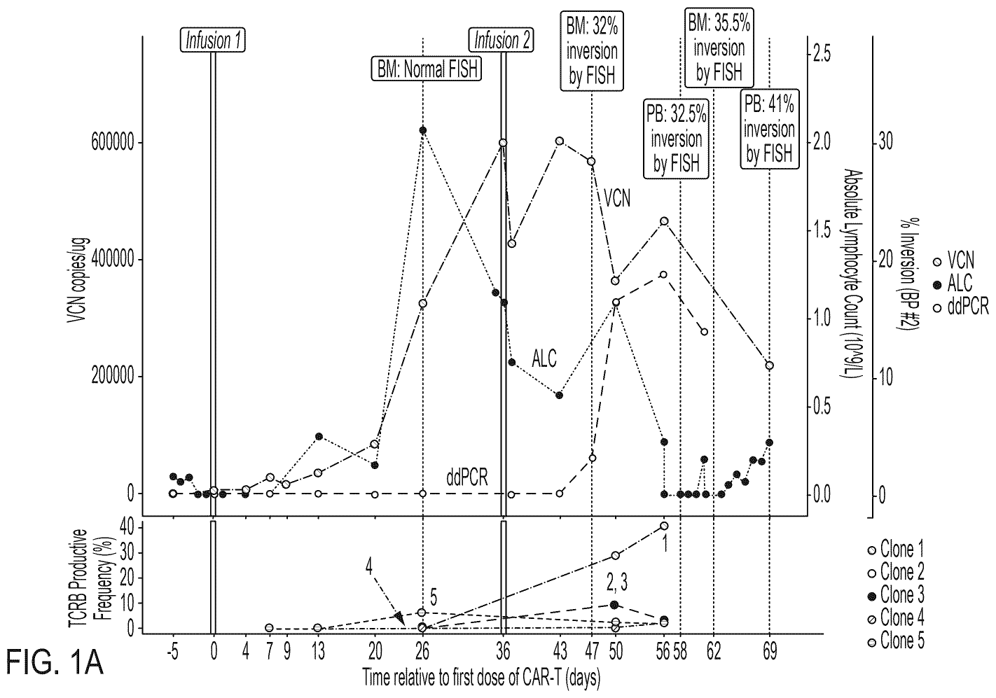

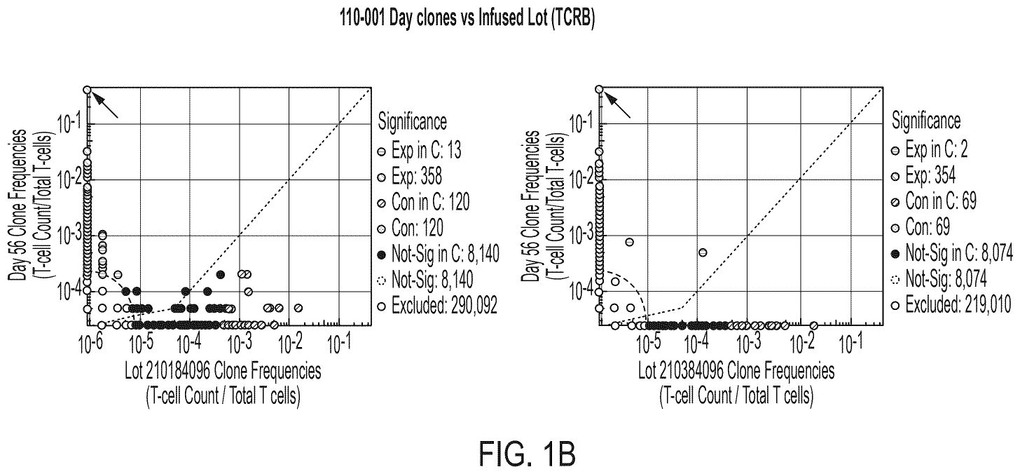

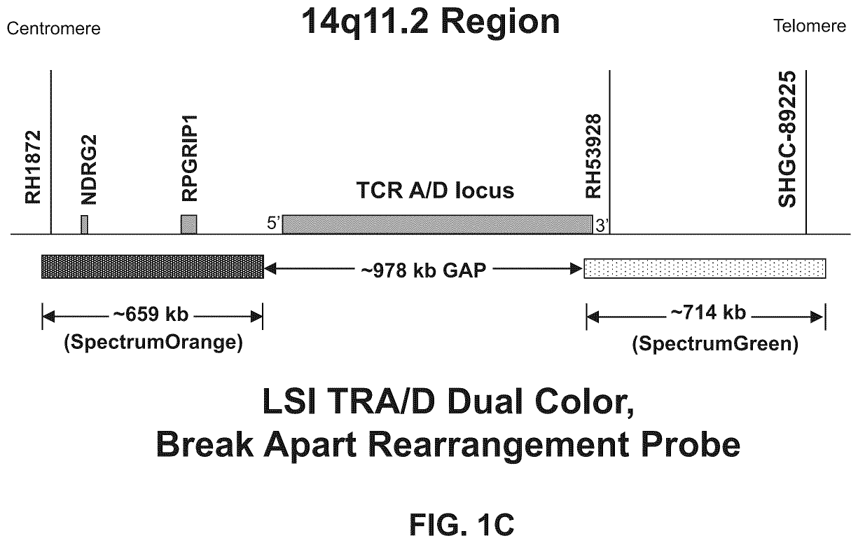

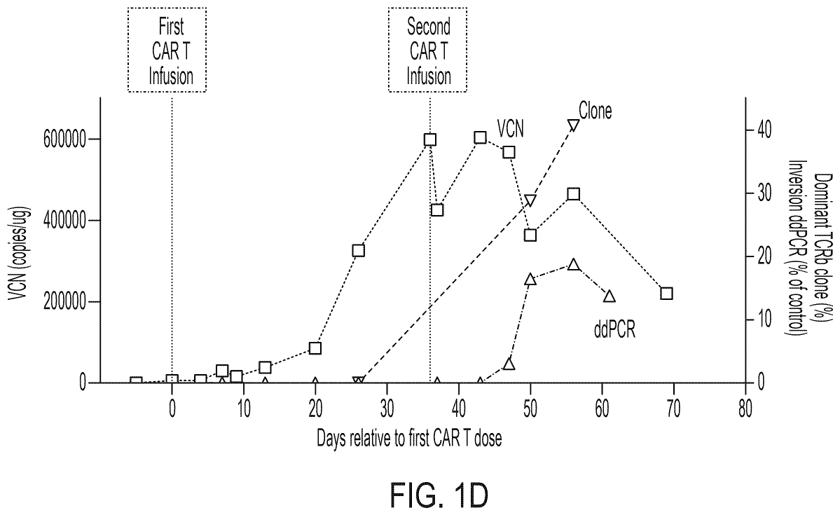

A chromosome 14 inversion was found in a patient who developed bone marrow aplasia following treatment with allogeneic chimeric antigen receptor (CAR) T cells containing gene edits made with transcription activator-like effector nucleases (TALENs). Sequencing of the patient sample revealed that the centromeric breakpoint did not involve the TALEN editing site at the T cell receptor α constant (TRAC) locus on chromosome 14 but instead involved a site approximately 10 kb distal at the T Cell Receptor Alpha Joining 7 (TRAJ7) locus, and the telomeric breakpoint was found at the Immunoglobulin Heavy Variable (IGHV) 3-69-1 locus. Recombination signal sequences (RSS) were found at the sites of inversion suggesting recombination activating gene (RAG)-mediated activity. While the chromosome 14 inversion represented a dominant clone, it was detected in the context of decreasing absolute CAR T and overall lymphocyte counts (FIG. 1D). The inversion was not detected in the drug product administered to this patient nor in any drug product used in this or related trials. The inversion was also not detected in this patient at earlier timepoints and it was not seen in any other patient, enrolled in this or related trials. There were no clinical consequences associated with the inversion. This case illustrates that spontaneous recombination events, likely mediated by RAG enzymes and unrelated to gene editing can occur in adoptive therapy studies.

A female patient with treatment-refractory transformed follicular lymphoma who failed production for an autologous CAR T cell product was enrolled in an allogeneic CD19 CAR T cell study (NCT04416984: ALPHA-2; safety and efficacy of ALLO-501A anti-CD19 allogeneic CAR T cells in adults with relapsed/refractory large B cell lymphoma). The CAR T cells were gene edited at the TRAC locus on chromosome 14 to prevent T cell receptor (TCR) expression and avoid graft versus host disease (GvHD), and at the (D52 locus on chromosome 1 to allow lymphodepletion with an anti-CD52 antibody. Following lymphodepletion with fludarabine, cyclophosphamide and an anti-CD52 antibody, the patient received one infusion of CAR T cells from a male donor on day 0 with a resulting partial response and another dose of CAR T cells on day 36 (planned as part of a consolidation regimen) from a second male donor without additional lymphodepletion. Prior to the second infusion the patient underwent bone marrow biopsy on day 26 due to cytopenia, a common side effect observed with CAR T therapy (Nahas, G. R. et al. Incidence and risk factors associated with a syndrome of persistent cytopenias after CAR-T cell therapy (PCTT). Leuk Lymphoma 61, 940-943, doi: 10.1080/10428194.2019.1697814 (2020); Rejeski, K. et al. CAR-HEMATOTOX: a model for CAR T-cell-related hematologic toxicity in relapsed/refractory large B-cell lymphoma. Blood 138, 2499-2513, doi: 10.1182/blood.2020010543 (2021)). Trilineage hematopoiesis with a cellularity of 30% was observed in the biopsy with some normal female lymphocytes in addition to male donor lymphocytes. CAR T expansion was observed following the first infusion, peaking at day 36, followed by contraction, then a subsequent and comparable peak following the second dose of CAR T cells, and subsequent secondary contraction (FIG. 1A). This profile is expected and typical of CAR T cell expansion/contraction observed in other patients treated with the same product.

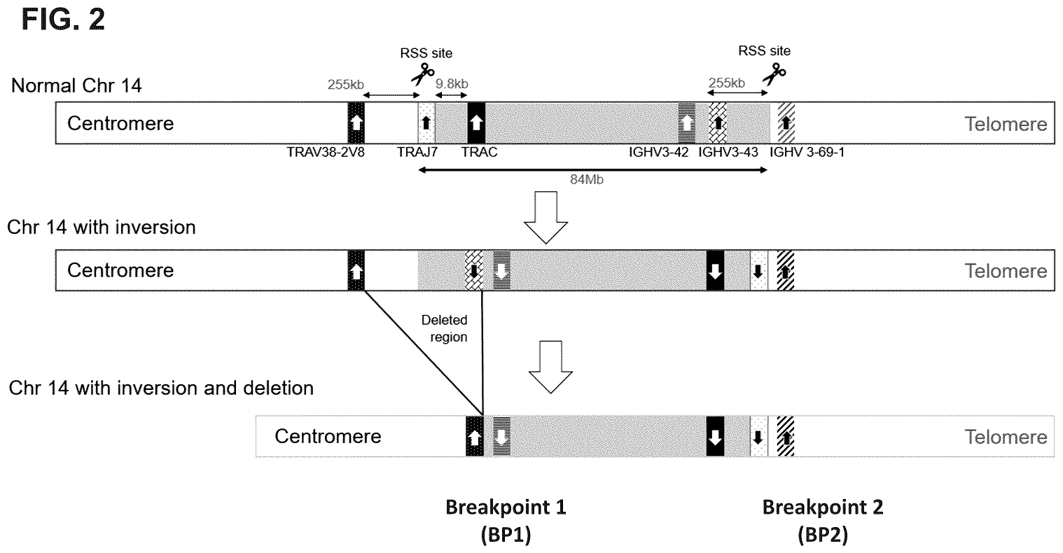

FIG. 1A shows longitudinal measurement of CAR T cell VCN, ALC and TCRβ clones in the patient. The primary y-axis (left) captures CAR T cells measured by VCN [teal line—labelled “VCN”], and the secondary y-axis (right) captures levels of ALC [purple line-labelled “ALC”]. Bottom panel shows the 5 most abundant TCRβ clones at Day 56. The VCN rose after the first infusion at Day 0, peaks first at Day 36 and then again at Day 43. The rearrangement was first detected at Day 47, in the context of falling VCN and ALC. The proportion of cells positive for the rearrangement by FISH rose through day 69 as the VCN continued to decline. The TCRβ clone detected at Days 50 and 56 with frequencies of 29% and 41%, respectively, is assumed to be the clone carrying the inversion given its similarity in abundance and trajectory to the event identified by the FISH assay. The inversion junction (for telomeric breakpoint or “Breakpoint 2”-see FIG. 2) was measured by ddPCR relative to a control gene to estimate relative abundance of the total lymphocyte population and is presented in red (red line-labeled “ddPCR”).

The patient developed HHV6 reactivation on day 30 and underwent anti-viral therapy, potentially contributing to the observed cytopenias. A repeat bone marrow evaluation at day 47 showed a chromosome (14) (q11.2q32) inversion in all donor-derived T cells (19 out of the total 20 cells assayed via karyotyping: data not shown). The cells carrying the chromosome 14 inversion in the peripheral blood were traced back to the first infused allogeneic drug product lot via human leukocyte antigen (HLA) and short tandem repeat (STR) multiplex genomic typing which uniquely identify recipient and donor alleles. This showed that 98% of the T cells were from the first donor infusion and the remaining 2% of the cells present were of host patient origin, without any cells from the second donor infusion. Repeat karyotyping was conducted on bone marrow from day 62 but failed due to inadequate mitotic activity (no metaphase cells to analyze). A peripheral blood analysis on day 69 yielded only 4 mitotic cells which showed the presence of the inversion. The difficulty in obtaining metaphase cells at both time points indicates that the remaining cells had a low proliferative index.

Results of a TCR A/D break-apart fluorescence in situ hybridization (FISH) assay (Vysis TRA/D Break Apart FISH Probe Kit-ACUTE LYMPHOCYTIC LEUKEMIA (ALL)—using a SpectrumOrange probe spanning approximately 659 kb centromeric of the T-cell receptor alpha/delta locus and a SpectrumGreen probe spanning approximately 714 kb telomeric of the T-cell receptor alpha/delta locus.—FIG. 1C), which does not require actively dividing cells, showed rearrangement at the TCR A/D locus in between 32 and 41% of 200 cells sampled at various time points (Table 1A).

TABLE 1A

| | | | | Chromo- | | |
|---|---|---|---|---|---|---|
| | | ddPCR | | some | | |
| | | % | % 14q11 | Inversions | | |
| | | Telo- | TCRA/D | in Male | VCN | |
| | Sample | meric | FISH- | Cells by | (copies/ | ALC |
| Visit¹ | Type | Inversion | Positive² | Karyology³ | μg) | (10⁹/L) |
| day -5 | Peripheral | 0 | NE | NE | 0 | 0.10 |
| | blood | | | | | |
| day 0 | Peripheral | 0 | NE | NE | 6386 | 0 |
| (post- | blood | | | | | |
| dose) | | | | | | |
| day 4 | Peripheral | Failed | NE | NE | 5900 | 0 |
| | blood | due to | | | | |
| | | technical | | | | |
| | | issues | | | | |
| day 7 | Peripheral | 0 | NE | NE | 30308 | 0 |
| | blood | | | | | |
| day 9 | Peripheral | Failed | NE | NE | 16159 | — |
| | blood | due to | | | | |
| | | technical | | | | |
| | | issues | | | | |
| day 13 | Peripheral | 0 | NE | NE | 38288 | 0.33 |
| | blood | | | | | |
| day 20 | Peripheral | 0 | NE | NE | 85408 | 0.17 |
| | blood | | | | | |
| day 26 | Bone | NE | Not | 0/5 | 360534 | — |
| | marrow | | detected | | | |
| day 26 | Peripheral | 0 | NE | NE | 325661 | 2.07 |
| | blood | | | | | |
| day 36 | Peripheral | NE | NE | NE | 598680 | 1.09 |
| | blood | | | | | |
| day 37 | Peripheral | 0 | NE | NE | 425444 | 0.75 |
| | blood | | | | | |
| day 43 | Peripheral | 0 | NE | NE | 604146 | 0.56 |
| | blood | | | | | |
| day 47 | Peripheral | 3 | NE | NE | 567900 | |
| | blood | | | | | |
| day 47 | Bone | NE | 32 | 19/19 | NE | — |
| | marrow | | | | | |
| day 50 | Peripheral | 16 | NE | NE | 363429 | 1.08 |
| | blood | | | | | |
| day 56 | Peripheral | 19 | NE | NE | 465473 | 0.30 |
| | blood | | | | | |
| day 58 | Peripheral | NE | 33 | NE | NE | 0 |
| | blood | | | | | |
| day 61 | Peripheral | 14 | NE | NE | NE | 0 |
| | blood | | | | | |
| day 62 | Bone | 25 | NE | NE | NE | NE |
| | marrow | | | | | |
| | DNA | | | | | |
| day 62 | Bone | NE | 36 | Failed | NE | NE |
| | marrow | | | due to | | |
| | | | | insufficient | | |
| | | | | mitotic | | |
| | | | | cells | | |
| | | | | (0/0⁴) | | |
| day 69 | Peripheral | NE | 41 | 4/4 | 220346 | 0.3 |
| | blood | | | | | |

Detection timepoints
ND = Not detected;
NE = ddPCR, FISH, or karyology were not evaluated.
¹Patient had a 6-day delay in 2nd infusion. Visit dates are calculated based on this 6-day delay.
²Fresh bone marrow and peripheral blood samples were analyzed using the 14q11 TCRA/D FISH assay at the clinical site (Medical College of Wisconsin).
³Karyotype = number of dividing cells, male with chromosome 14 inversion/male dividing cells out of 20.
⁴No metaphase cells were detected.

At all timepoints, karyotype and FISH analysis findings occurred in the context of declining CAR T cells, as measured by vector copy number (VCN) analysis (FIG. 1A), and low peripheral blood counts with absolute lymphocyte count (ALC) peaking at 2.07×10³ cells/ul on day 28, declining to 1.08×10³ cells/ul by day 44 and to 0.3×10³ cells/ul by day 51. Although chromosome (14) (q11.2q32) inversion is a feature of T cell prolymphocytic leukemia (T-PLL), diagnosis of T-PLL was ruled out due to absence of all other diagnostic criteria including the expression of other genetic abnormalities and the presence of a high T cell count, organ involvement, or expression of T cell leukemia/lymphoma protein (TCL1) (Staber, P. B. et al. Consensus criteria for diagnosis, staging, and treatment response assessment of T-cell prolymphocytic leukemia. Blood 134, 1132-1143, doi: 10.1182/blood.2019000402 (2019)). Additionally, the rare lymphocytes observed in the bone marrow were consistent with CAR T cells by morphology and by immunophenotype and did not have blast morphology.

Clonality analysis using TCRβ sequencing showed a clone with 29% and 41% abundance at days 50 and 56, respectively (FIG. 1A). This clone was not detected at the previous sampling point of day 26. A total of 8 clones with lesser abundances ranging from 1% to 3% were identified at day 56 and all other clones were present with an abundance of less than 1%. Given the similarity in abundance of the dominant TCRβ clone to that of the 14q11disruption by FISH, this clone was assumed to be the clone containing the inversion. This dominant TCRβ clone could not be detected in the drug product above the level of detection of the assay (FIG. 1B).

FIG. 1B demonstrates that the dominant clone from the patient was not detected in the ALLO-501A Drug Product. TCRβ sequencing results are shown from the patient's peripheral blood on day 56 (y-axis) and drug product (x-axis). When the points all align on the axes it indicates that the two repertoires are distinct, and when the points align on the diagonal it indicates that the repertoires are highly similar. Points that fall on the axes are not detected in the other sample, for example points along the y-axis in the patient were not detected in the infusion product. The dominant clone (41%) in the patient is observed as the top left dot on the y-axis, indicating that it was not detected in the infusion product. The shades of points represent the statistical significance of that clone's shift in frequency. Clones labeled as “Pt” have significantly higher frequency in the patient's peripheral blood sample compared to the GMP infusion product lot (significance assessed through a binomial distribution framework), while the clones labeled as “Lot” represent significantly higher frequency in the infusion product lot compared to the patient's sample. The two shades of grey indicate that there was either no statistical difference detected between the two frequencies (dark gray circles) or untested (or excluded, light gray circles within the dashed quarter circle). Lastly, the dark circle around the outside of the points represents that the clone was in the infusion product (all clones not along the y-axis are encircled). TCRβ sequencing was used to track the top clones from the patient's peripheral blood (PB) over time and infused drug product lots. The top 20 clones were analyzed to determine the proportion or frequency of each clone in the total repertoire of the following samples: patient PB at day 56 (left), drug product Lot 1 and drug product Lot 2 (data not shown). The dominant clone from the patient sample was observed at day 50 and day 56 but not in any other samples, including the two drug product lots (data not shown).

As shown in FIG. 1D, the detection of the inversion junction via ddPCR coincides with the emergence of the dominant TCRβ clone. The inversion was not detected by ddPCR in the patient before day 47, in any other patients on this or any other Allogene trial, in any other drug product, or in any donor source.

Short and long-read whole genome sequencing of a day 61 peripheral blood sample was conducted with next-generation sequencing (NGS) to examine the precise breakpoints of the inversion and determine whether the inversion could be a consequence of TALEN editing. Sequencing confirmed the presence of the chromosome 14 inversion and identified the inversion breakpoint sites (FIG. 2). The centromeric (14q11.2) inversion site occurred within the TCR A/D locus at the start of the T cell receptor joining gene TRAJ7 (chr14: 22537626; hg38 reference genome build). The telomeric (14q32) inversion site occurred at the IGHV3-69-1 pseudogene (chr14: 106728167; hg38 reference genome build) within the IGH variable region.

FIG. 2 shows a map of chromosome 14 inversion detected and characterized from peripheral blood sample of the patient at Day 61 by short and long-read whole genome sequencing. The two breakpoints were identified at TRAJ7 and IGHV3-69-1. The relative position and approximate distances from the TRAC TALEN cut site are shown. Also marked are predicted RAG cleavage sites, as inferred from RSS sequences, at each of the two breakpoints. Other genes are added as placemarkers to show the impact of inversion and deletion.

The TRAJ7 site of the inversion within the TCR A/D locus is approximately 10 kb from the TRA (locus where TALEN gene editing takes place, indicating that the inversion was not generated by on-target cleavage from the TALEN. Large deletions have been reported to occur during the gene editing process (Adikusuma et al. (2018). Large deletions induced by Cas9 cleavage. Nature 560: E8-E9; Kosicki et al. (2018). Repair of double-strand breaks induced by CRISPR-Cas9 leads to large deletions and complex rearrangements. Nat Biotechnol 36:765-771; Park et al. (2022). Comprehensive analysis and accurate quantification of unintended large gene modifications induced by CRISPR-Cas9 gene editing. Sci Adv 8: cabo7676) and may impact the formation of aberrant structural variants. Short and long read whole genome sequencing, however, only showed the expected 49 bp deletion at the TRAC TALEN on-target site and did not show evidence of large deletions at that locus. Furthermore, the presence of RSS sites at the inversion junction suggests that RAG-mediated recombination, and not large deletions at the TRAC TALEN site, is the likely cause of the inversion. The sequences within 500 bp of both breakpoints of the chromosome 14 inversion (q11.2q32) were analyzed to identify possible off-target TALEN binding sites that could have been involved. Results showed no potential TRAC or CD52 homo- or hetero-dimer TALEN binding sites indicating that this inversion is also not associated with TALEN off-target gene editing. No potential TALEN binding sites were found at levels less than or equal to 7 accumulated mismatches across both 15 bp monomer DNA binding sites. No lentiviral vector integration site was detected at or near the site of inversion.

Consensus RSSs were observed at both sites of inversion. RSSs are typically comprised of a conserved heptamer (consensus 5′-CACAGTG-3′) and nonamer (consensus 5′-ACAAAAACC-3′) sequence separated by either 12 (RSS-12) or 23 (RSS-23) nucleotides of variable sequence. The presence of RSS supports the hypothesis that the inversion was a consequence of RAG mediated V (D) J recombination between the 2 distal sites. That is, the centromeric TRAJ7 harbors an adjacent RSS-12 sequence (chr14: 22,537,594-22,537,621; hg38 reference genome build) and the telomeric IGHV3-69-1 contains an adjacent RSS-23 sequence (chr14: 106, 728, 124-106, 728, 162; hg38 reference genome build), both conforming to the 12/23 rule (van Gent et al. The RAG1 and RAG2 Proteins Establish the 12/23 Rule in V(D)J Recombination, Cell 85 (1), 107-113, 10.1016/s0092-8674 (00) 81086-7 (1996)). Deletions following the breaks and prior to ligation at TRAV38-2DV8 and TRAJ7, as well as at IGHV3-42 and IGHV3-69-1 were also observed. It has been observed that the insertion of the signal end fragment generated by the RAG proteins nearly always produces deletions or other rearrangements of target DNA (Chatterji, M., Tsai, C. L. & Schatz, D. G. Mobilization of RAG-generated signal ends by transposition and insertion in vivo. Mol Cell Biol 26, 1558-1568, doi: 10.1128/MCB.26.4.1558-1568.2006 (2006)). Short-read sequencing was used to identify the two breakpoints for the inversion and subsequent long-read sequencing was used to identify the deletion at each of the two breakpoints.

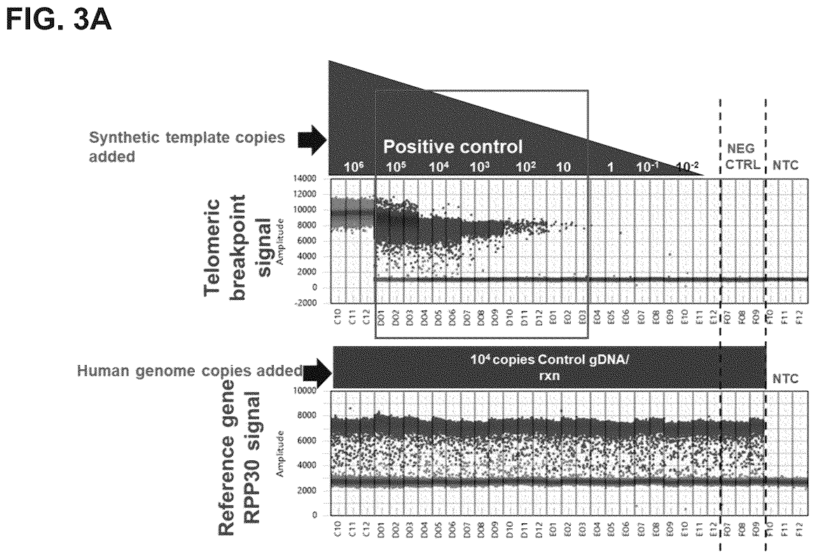

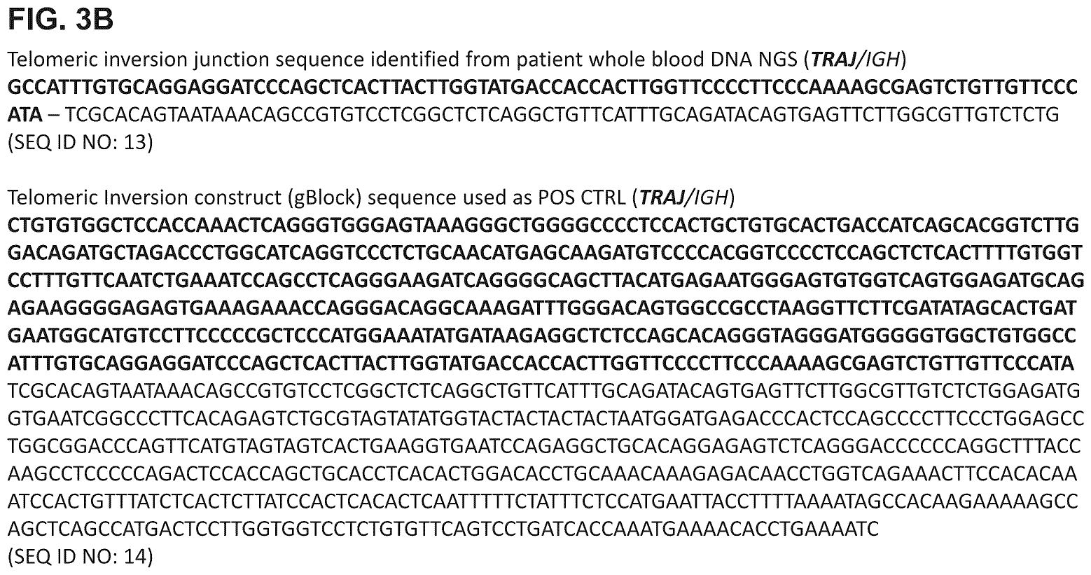

Sequence information from the short-read NGS analysis was used to design primer pairs to detect both ends of the inversion in order to quantitatively assess the level of inversion present in the patient sample, and in clinical lots and their corresponding PBMCs prior to TALEN editing (FIG. 3A). The assay had a lower limit of detection of 1 in a 1,000, which is consistent with reported estimates for digital droplet polymerase chain reaction (ddPCR) sensitivity (Dong, L., Wang, S., Fu, B. & Wang, J. Evaluation of droplet digital PCR and next generation sequencing for characterizing DNA reference material for KRAS mutation detection. Sci Rep 8, 9650, doi: 10.1038/s41598-018-27368-3 (2018)).

FIG. 3A-3B depicts the method of development of a telomeric inversion junction ddPCR assay. As part of the initial method development, primers and probes for the ddPCR assay were designed and optimized. Amplified copies of inversion junctions and RPP30 were measured by a ddPCR droplet reader to analyze percent inversion. Each ddPCR plate consisted of a negative control (NEG CTRL: TaqMan™ Control Genomic DNA), positive control (POS CTRL: Synthetic template was spiked into the TaqMan™ Control Genomic DNA), and a no template control (NTC: water). The frequency of inversion was measured as percentage of the total genome copies of the reference gene (RPP30). The synthetic template was titrated into 35 ng of human genomic DNA corresponding to ˜10,000 human genome copies and analyzed for specificity and sensitivity to the inversion sequence. Shown in FIG. 3A is the raw data from the ddPCR from the Quantasoft software indicating the droplets observed for the telomeric breakpoint (blue-top panel) and the reference gene (green-bottom panel). The ddPCR assay was found to detect the signal from the synthetic template down to a sensitivity level of approximately 0.1% as shown by quantification of the copy numbers reported (Table 1B) (Dong, L. et al. 2018).

TABLE 1B
Limit of detection for ddPCR using synthetic template for telomeric inversion
breakpoint.

| | | | | Serial | | | | | | | |
|---|---|---|---|---|---|---|---|---|---|---|---|
| | | | | Dilutions | | | | | | | |
| | | | | of | | | | | | | |
| Reference | | | | Telomeric | | | | | | | |
| genomic | | | | Inversion | | | | % Telomeric | | | |
| DNA* | | | | Template | Telomeric breakpoint | | | breakpoint relative to | | | |
| Expected | RPP30 (Copies/Reaction) | | | Expected | (Copies/Reaction) | | | RPP30 | | | Data |
| copies** | R1 | R2 | R3 | copies | R1 | R2 | R3 | R1 | R2 | R3 | reporting |
| 10000 | 8393.4 | 8564.9 | 8401.6 | 10000 | 7360.6 | 7503.4 | 7590.7 | 87.7 | 87.6 | 90.3 | Detected, |
| | | | | | | | | | | | report |
| | | | | | | | | | | | values |
| 10000 | 8624.2 | 8349.8 | 8463.0 | 1000 | 844.2 | 811.8 | 754.4 | 9.8 | 9.7 | 8.9 | Detected, |
| | | | | | | | | | | | report |
| | | | | | | | | | | | values |
| 10000 | 8329.2 | 8461.2 | 8450.1 | 100 | 84.0 | 69.1 | 67.0 | 1.0 | 0.8 | 0.8 | Detected, |
| | | | | | | | | | | | report |
| | | | | | | | | | | | values |
| 10000 | 8567.4 | 8825.2 | 8555.2 | 10 | 7.7 | 10.6 | 2.6 | 0.1 | 0.1 | 0.03 | Detected, |
| | | | | | | | | | | | report |
| | | | | | | | | | | | values (R1 |
| | | | | | | | | | | | and R2) |
| 10000 | 8496.0 | 8619.7 | 8255.0 | 1 | 0 | 0 | 2.5 | 0 | 0 | 0.03 | ND |

*35 ng of human genomic DNA used;
ND = Not detected;
% Inversion < LOD (0.1%);
R1, R2, and R3 refer to three replicates per sample;
**Expected copies of RPP30

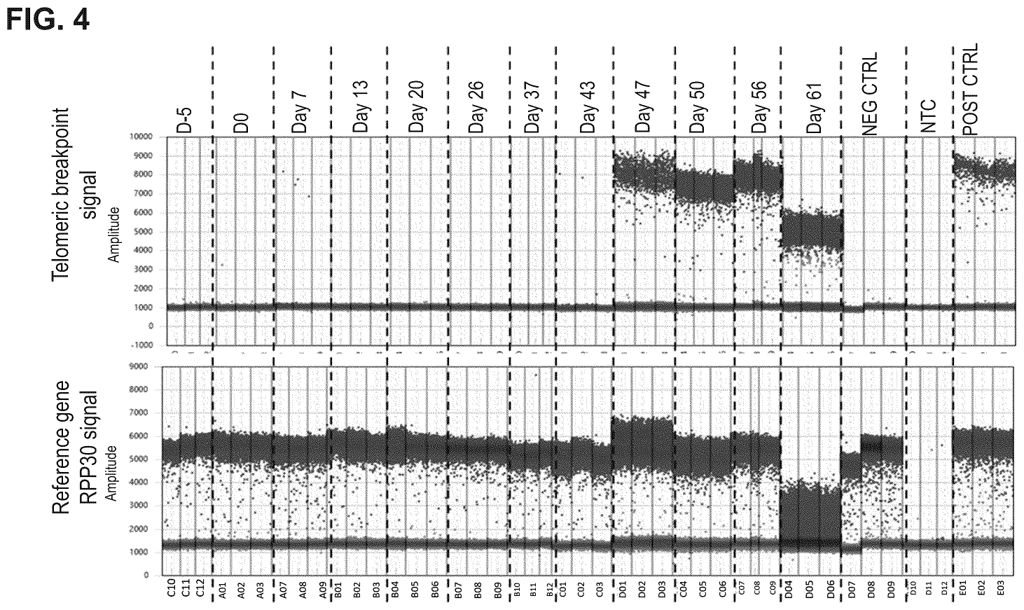

The available frozen peripheral blood samples from this patient, all other patients dosed with the drug product, and donor material were analyzed with the ddPCR 15 assay for the telomeric end of the inversion at timepoints spanning a period from prior to cell dosing up to day 61 (FIG. 4).

FIG. 4 shows longitudinal detection of the telomeric breakpoint junction by ddPCR in patient peripheral blood samples. The x-axis represents patient samples (in triplicate), and the y-axis denotes the amplitude of the signal. Each dot represents an independent droplet measurement event. The upper panel shows the detection of the telomeric breakpoint and the lower panel shows detection of the reference signal. The lower signal amplitude on day 61 may be due to the presence of lower quality DNA in this sample.

The earliest timepoint at which the inversion was detected was 47 days after CAR T cell infusion. The frequency of inversion was measured on a relative scale as percentage compared to the reference gene, Ribonuclease P/MRP Subunit P30 (RPP30), and ranged from 3% to 19% between day 47 and day 61 in the peripheral blood (FIG. 4 and Table 1A). The longitudinal patient samples were also assayed by ddPCR for the centromeric inversion junction with detection at the same time points (data not shown). The time-course of inversion detection is concordant with detection via karyology, FISH, and emergence of the implicated clone by TCRβ sequencing. It decreased in concordance with VCN and ALC by day 60 (FIG. 1 and Table 1A). Samples from other patients were tested at 2 timepoints spanning dosing and ranging from lymphodepletion day-7 through 9 months and the chromosome 14 inversion was below the limit of detection in all cases. No other drug products or healthy donor starting materials for drug products tested positive across all trials using the gene editing manufacturing platform.

The chromosome 14 inversion was initially described in T cell tumor lines (Baer et al. (1985). Fusion of an immunoglobulin variable gene and a T cell receptor constant gene in the chromosome 14 inversion associated with T cell tumors. Cell 43:705-713; Baer et al. (1987). The mechanism of chromosome 14 inversion in a human T cell lymphoma. Cell 50:97-105; Baer et al. (1987). The breakpoint of an inversion of chromosome 14 in a T-cell leukemia: sequences downstream of the immunoglobulin heavy chain locus are implicated in tumorigenesis. Proc Natl Acad Sci USA 84:9069-9073; Denny et al. (1986). A chromosome 14 inversion in a T-cell lymphoma is caused by site-specific recombination between immunoglobulin and T-cell receptor loci. Nature 320:549-551); however, it was later shown to be present in normal T cells from healthy individuals. Chromosome 14 inversions have been detected in post-thymic, mature T cells of normal individuals and is believed to be mediated by V (D) J recombination machinery (Callen, E. et al. Chimeric IgH-TCRalpha/delta translocations in T lymphocytes mediated by RAG. Cell Cycle 8, 2408-2412, doi: 10.4161/cc.8.15.9085 (2009); Machado, H. E. et al. Genome-wide mutational signatures of immunological diversification in normal lymphocytes. bioRxiv, 2021.2004.2029.441939, doi: 10.1101/2021.04.29.441939 (2021)). In one report, in the 3 healthy donors studied by karyotype analysis (>1,000 cells analyzed per donor) the inversion was present in all donors at an average of 0.15% of lymphocytes analyzed (Aurias, A. et al. Inversion (14) (q12qter) or (q11.2q32.3): the most frequently acquired rearrangement in lymphocytes. Hum Genet 71, 19-21, doi: 10.1007/BF00295660 (1985)). In another study of 2,595 healthy individuals (20-40 cells per individual analyzed), 49 chromosomal breakpoints were detected involving either 14q11 or 14q32. Six of these infrequent rearranged lymphocytes out of a total of 53,580 cells analyzed showed a chromosome 14q11: q32 inversion (Hecht, F. et al. Fragile sites limited to lymphocytes: molecular recombination and malignancy. Cancer Genet Cytogenet 26, 95-104, doi:10.1016/0165-4608 (87) 90137-3 (1987)). Studies examining the integrity of chromosomes in normal cultured lymphocytes have shown 4 frequent sites of chromosome breakage: 7p13 (near location of TCRγ chain), 7q35 (location of TCRβ chain), 14q11 (location of TCRα chain) and 14q32 (location of IGH chain) and suggest that these act as fragile sites specifically in normal lymphocytes as defined by the non-random frequency of chromosomal translocations or inversions associated with them (Hecht, F. (1987); Welch, J. P. et al. Non-random occurrence of 7-14 translocations in human lymphocyte cultures. Nature 255, 241-245, doi: 10.1038/255241a0 (1975)).

In summary, TALEN gene editing was not implicated in the chromosome inversion event since sequencing indicates that the inversion did not occur at TALEN sites. While the source of the chromosome structural variant was the infused donor cells, the variant was only detected after product infusion and the sites of inversion did not involve TALEN-mediated gene editing. Similarly, lentiviral vector integration did not contribute to the generation of the chromosome 14 inversion. The inversion was associated with RSS indicating that they were a consequence of V (D) J recombination. In addition to RAG mediated gene rearrangement during T cell development, reactivation of RAG post-thymically, a process termed TCR revision, has been demonstrated in preclinical studies. (Mostoslavsky, R. & Alt, F. W. Receptor revision in T cells: an open question? Trends Immunol 25, 276-279, doi: 10.1016/j.it.2004.04.001 (2004)). This has been speculated to increase T cell repertoire in the event of a dominant T cell response and may play a role in peripheral tolerance.

The clonal population containing the inversion arose in the context of an overall contraction of all donor cells in the patient, as seen in other CAR T cell trials (Nobles, C. L. et al. CD19-targeting CAR T cell immunotherapy outcomes correlate with genomic modification by vector integration. J Clin Invest 130, 673-685, doi: 10.1172/JCI130144 (2020); Sheih, A. et al. Clonal kinetics and single-cell transcriptional profiling of CAR-T cells in patients undergoing CD19 CAR-T immunotherapy. Nat Commun 11, 219, doi: 10.1038/s41467-019-13880-1 (2020)). The fact that none of the T cells detected were from the second infusion was unexpected, but could perhaps be explained by hostile marrow conditions which resulted in aplasia, with the first infusion product still declining and the second one not engrafting sufficiently. The inversion was not detected in donor or the clinical lots used to treat the patient, or from any other clinical batch or patient sample, leading to the conclusion that this event arose post infusion and is isolated to this particular patient. While the role of the inversion in clonal expansion is uncertain, there is no evidence that the expansion was the consequence of the inversion; similarly, since there was no expansion of CD3 positive grafted cells, a TCR-driven expansion is also not supported. Importantly, there was no evidence of clinically significant sequelae related to the inversion and specifically there was no evidence of malignant transformation. After the CAR T cell treatment, the patient underwent allogeneic transplant due to prolonged pancytopenia on day 76 and showed immune reconstitution before experiencing disease relapse and starting new therapy on day 134. However, it was not possible to monitor for any long-term consequences of the inversion due to the patient's death from disease progression.

With the advent of adoptive cell therapy, there has been a rapid expansion of industry and/or academic led clinical programs that employ gene editing or engineering cell products, both of which carry a risk of genetic alternation such as off-site gene editing, chromosomal structural changes (large deletions, inversions, or translocations) or insertional mutagenesis. This report illustrates that structural variants also occur naturally in human lymphocytes in vivo and can be expected to be observed in the clinic going forward. In a recent publication, 1,037 structural variants were observed across 635 normal human lymphocytes. Of these structural variants, 85% involved either the Ig or TCR regions. Furthermore, they go one to show that an estimated 12% of non-Ig-TCR and 84% of IG-TCR structural variants were RAG-mediated, due to the presence of an RSS motif or heptamer being proximal to the breakpoint (Machado et al. (2022). Diverse mutational landscapes in human lymphocytes. Nature 608:724-732).

### Methods

Patient treatment history. The patient was a 57-year-old woman with Stage IV transformed follicular lymphoma (tFL) with a t (8; 14) chromosomal translocation involving cMyc rearrangement. She was refractory to all previous therapies and had undergone radiation to mesenteric lymph nodes and was also unable to undergo autologous CAR T cell therapy due to non-expansion of autologous CAR T cells during manufacturing. She received cyclophosphamide 500 mg/m²/day, fludarabine 30 mg/m²/day×3 days on days −5, −4, and −3 and ALLO-647 (anti-CD52 antibody, Allogene Therapeutics, South San Francisco, CA) 20 mg/day×3 days on day −2, −1, and 0 and received an ALLO-501A dose of 120×10⁶ CAR cells on day 0. A second dose of ALLO-501A, termed consolidation, was administered on day 36 from an alternate batch without preconditioning due to recent grade 3 HHV-6 reactivation and subsequent pancytopenia. Both cell infusions were from separate male donor-derived T cells. Despite being refractory to all prior therapy, she achieved a partial response on day 28, which was confirmed on day 56. The patient underwent allogeneic transplant due to prolonged pancytopenia on day 76 and showed immune reconstitution before experiencing disease relapse and starting further therapy on day 134.

Karyotyping and FISH. Standard karyotyping was performed locally by the Medical College of Wisconsin (Milwaukee, WI) on tissue from fresh peripheral blood and bone marrow samples. The 14q11.2 region was further interrogated by fluorescence in situ hybridization (FISH) using TRA/D Dual Color Break Apart probes (FIG. 1C; Abbott Molecular, Abbot Park, IL), with Spectrum Orange Probe: chr14: 20895431-21554322 and Green Probe: chr14: 22531141-23244993 (hg38 reference genome build). The break-apart probe sets were applied to specimens, hybridized, and washed according to the standard Medical College of Wisconsin interphase FISH protocol. All cells present in the specimen were assayed, until a full 20-cell study (if able) was obtained; karyotyping (cut apart and pairing of the chromosomes) was conducted in ˜3 cells per cell line. A total of 200 interphase cells were scored to determine the TCRA/D rearrangement.

HLA and STR typing. Human leukocyte antigen (HLA) and chimerism testing were performed by Versiti (Milwaukee, WI) on ALLO 501A drug products (i.e., CAR T cells) and patient genomic DNA. Eleven HLA class I and II sequences were amplified and sequenced with high resolution next-generation sequencing (NGS). Additionally, multiplex PCR amplification was conducted on 8 total short tandem repeat (STR) loci. Amplification products from CAR T cells and patient baseline DNA were used to uniquely identify recipient and donor alleles, and fluorescence of recipient and donor alleles were used to calculate the percent chimerism. Using the most appropriate identified markers, the average percentages derived from the recipient and donor were reported.

TCRβ clonality data. Sequencing of the TCRβ CDR3 region in genomic DNA extracted from whole blood or ALLO-501A drug product was performed by Adaptive Biotechnologies (Seattle, WA). Clone frequencies for each sample were calculated as the number of reads representing a unique CDR3 sequence divided by the total reads for the sample. Scatterplots were used to compare clone frequencies between 2 samples. The sensitivity of the assay was <7 in 1 million clones, 95% CI (0, 6.7× 10⁻⁶) (FIG. 1B). Statistical significance of a clone's shift in frequency in one sample relative to the other was calculated using a binomial distribution framework.

VCN analysis. Vector copy number analysis was performed and analyzed by Navigate Biosciences (Carlsbad, CA) utilizing a validated qPCR assay. Genomic DNA was extracted from patient peripheral blood and bone marrow aspirate samples collected in K2EDTA tubes utilizing the QIAMP Blood DNA Midi Kit (QIAGEN, Germantown, MD) and quantified using Qubit Fluorometer. Patient DNA input ranged from 20-200 ng per reaction and was analyzed with a validated primer probe set specific for the ALLO-501A drug product, 2× TaqMan®Gene Expression Master Mix (Thermo Fisher Scientific, Waltham, MA) and Molecular-grade water, Ultrapure Distilled water, DNase and RNase-free water, (Thermo Fisher Scientific). Analysis was conducted on the Applied Bioscience ViiA7 qPCR system in conjunction with the ViiA7 Applied Biosystems software version 1.2.4 or newer (Thermo Fisher Scientific).

Off-target TALEN assessment. Flanking sequences (˜500 bp) on each side of the breakpoints were screened for potential TRAC and CD52 TALEN binding off-target sites (4 sequences, 2 from each pair) using publicly available tools (Juillerat, A. et al., et al. (2014). Comprehensive analysis of the specificity of transcription activator-like effector nucleases. Nucleic Acids Res. 42, 5390-5402). Sequence analysis was performed using 3 approaches: (1) NCBI BLASTn suite optimized for more dissimilar sequences, (2) Fuzzy Search DNA, which accepts a DNA sequence along with a query sequence and returns sites that are identical or similar to the query, and (3) TALEN offer that scans input sequences for off-targets of a given TALEN recognition sequence. No potential TALEN binding sites were found with the methods described above at levels less than or equal to 7 cumulated mismatches across both 15 bp monomer DNA-binding sites, which essentially ruled out the potential for an off-target editing event in the breakpoint regions.

Whole genome sequencing and alignment. Peripheral blood was drawn from the patient into K2EDTA tubes on day 61 after infusion. Genomic DNA was purified from the blood samples using QIAamp DNA Blood kits (QIAGEN, Germantown, MD). Genomic DNA of ALLO-501A drug product (i.e., CAR T cells) was purified using AllPrep DNA Kit (mini) (QIAGEN, Germantown, MD). Libraries were prepared with Illumina DNA Prep kits (Illumina, San Diego, CA). Individual DNA libraries were sequenced using an Illumina NovaSeq6000 platform (Illumina) at SP-500 cycle, 2×250 bp paired end reads (Tarasov, A. et al., Sambamba: fast processing of NGS alignment formats. Bioinformatics 31, 2032-2034, doi: 10.1093/bioinformatics/btv098 (2015)). Sequencing was performed at a depth of 100× to ensure that any variants at as low as 1% frequency would be detected. Paired end sequencing tagged and sequenced both ends of the DNA fragments in the libraries, thus providing the ability to identify discordant alignments of paired reads to the genome to identify possible structural variants.

The alignment pipeline consists of a series of steps that started by evaluating the FastQ files for read quality with the FastQC online tool (version 0.11.8; Andrews, S. FastQC: A Quality Control Tool for High Throughput Sequence Data (2010)). Next, the reads were trimmed using Trim Galore 0.6.0 software (Babraham Institute Bioinformatics-Trim Galore) to remove sequencing adaptors and low-quality sequences. Pair-end reads >80 bp after trimming were aligned using the BWA software package (version 0.7.17; Li, H. & Durbin, R. Fast and accurate short read alignment with Burrows-Wheeler transform. Bioinformatics 25, 1754-1760, doi: 10.1093/bioinformatics/btp324 (2009)) and the generated alignment file was then sorted and indexed using SAMtools (version 1.9; Danecek, P. et al. Twelve years of SAMtools and BCFtools. Gigascience 10, doi: 10.1093/gigascience/giab008 (2021)). Finally, alignment statistics were generated using Picard (version 2.20.3; Broad Insitute), and a final QC report was generated using the MultiQC v1.8 (Ewels, P. et al., MultiQC: summarize analysis results for multiple tools and samples in a single report. Bioinformatics 32, 3047-3048, doi: 10.1093/bioinformatics/btw354 (2016)).

Alignment files generated during the whole genome sequencing (WGS) process were used as input for the identification of genomic structural variations. Duplicate reads, split reads (terminal sequence with >20 bp that do not match the reference sequence, and discordant reads that did not produce the expected alignment using SAMBLASTER (version 0.1.25; Faust, G. G. & Hall, I. M. SAMBLASTER: fast duplicate marking and structural variant read extraction. Bioinformatics 30, 2503-2505, doi: 10.1093/bioinformatics/btu314 (2014)) and Sambamba (version 0.7.1; Tarasov, A., 2015) were removed. The resulting files were used by 2 structural variation algorithms, LUMPY (version 0.2.13; Layer, R. M. et al., LUMPY: a probabilistic framework for structural variant discovery. Genome Biol 15, R84, doi: 10.1186/gb-2014-15-6-r84 (2014)) and Manta (version 1.6.0; Chen, X. et al. Manta: rapid detection of structural variants and indels for germline and cancer sequencing applications. Bioinformatics 32, 1220-1222, doi: 10.1093/bioinformatics/btv710 (2016)) to identify potential structural variation events with a minimum of 5 supporting sequencing reads. Results from both algorithms were combined and filtered to identify events affecting chromosome 14 with breakpoints in the proximity of the 2 FISH probes that suggested the existence of an inversion. The Integrative Genomics Viewer (IGV version 2.6.1; The Broad Institute) was used to validate possible structural variants and inspect the locations of potential inversion-related breakpoints.

Verification of breakpoint sequences. High coverage, long-read nanopore sequencing was used to verify the breakpoint sites, flanking sequence and structure of the chromosome 14 Inversion. Structural Variants (SVs) relative to GRCh38 reference genome assembly were detected using NGMLR pipeline. Reads spanning chromosome 14 q11.2 and q32 region were found. Two breakpoints (BP) were identified at 22,537,626 and 106,728,167, separately, matching the two BPs identified in the previous WGS with Illumina short-read sequencing. In addition, while one of the inverted junctions was intact, 2 deletions of approximately 255 kb each were identified at the other inverted junction. Molecular sequence of junction regions matched with expected reference genomic regions, although there were some small deletions, insertions and SNPs which were mostly due to base-calling errors from nanopore sequencing. Finally, amplicons spanning the breakpoints were generated and sequenced, confirming the breakpoint and flanking sequences.

Lentiviral insertion site analysis. Integration site analysis (ISA) was conducted at the Viral/Molecular High Density Sequencing Core at the University of Pennsylvania (Philadelphia, PA). This analysis was performed using the INSPIRED pipeline (Berry, C. C. et al. INSPIIRED: Quantification and Visualization Tools for Analyzing Integration Site Distributions. Mol Ther Methods Clin Dev 4, 17-26, doi: 10.1016/j.omtm.2016.11.003 (2017); Sherman, E. et al. INSPIIRED: A Pipeline for Quantitative Analysis of Sites of New DNA Integration in Cellular Genomes. Mol Ther Methods Clin Dev 4, 39-49, doi: 10.1016/j.omtm.2016.11.002 (2017)). Integrated lentiviral sequences from patient and ALLO-501A DP genomic DNA were analyzed through nested PCR amplification and subsequent sequencing on Illumina MiSeq or HiSeq platforms. Novel blocking locked nucleic acid primers were utilized to reduce sequencing of undesirable internal long terminal repeat sequences and integration site frequency was determined with the SonicAbundance method.

PCR method development. A linear double stranded DNA template (gBlock™) was designed as the synthetic template and positive control for method development for both the centromeric and telomeric sites of inversion based on the NGS sequence information derived from the patient (see FIG. 3B).

A ddPCR assay was developed for the specific TRAJ7/IGHV3-69-1 junction identified at Breakpoint 2 (BP2) (see FIG. 2) in the patient. Similar considerations may be applied to the analysis of other types of inversions or other chromosomal abnormalities. Primers and probes capable of detecting the specific TRAJ7/IGHV3-69-1 junction at BP2 were designed and a ddPCR assay specific for this inversion was developed in order to quantitatively assess its level (1) in the patient samples and other patients treated with the implicated drug product (DP) lot, (2) in the clinical DP lots, and (3) in their corresponding PBMCs prior to TALEN editing. The primers were optimized using a synthetic template derived from the NGS sequence information, as further described herein.

The primers and probe for the telomeric BP2 are shown in Table 1C below. The expected PCR product size is 232 bp.

TABLE 1C

| Telomeric | | |
|---|---|---|
| inversion | | SEQ ID |
| Breakpoint 2 | DNA sequence | NO: |
| Forward Primer | CCAGAGACAACGCCAAGAACTCA | 1 |
| Reverse Primer | CCCCGCTCCCATGGAAATATGA | 2 |
| Probe | CCACAGCCACCCCCATCCCT* | 3 |

*5′ FAM, 3′ ZEN/Iowa Black FQ Quencher.

In addition, a ddPCR assay was developed for the specific junction within the TRCA/D locus at the start of the T cell receptor joining gene TRAJ7 identified as Breakpoint 1 (BP1) (see FIG. 2) in the patient. Primers and probes capable of detecting the specific junction at BP1 were designed and a ddPCR assay specific for this inversion was developed in order to quantitatively assess its level (1) in the patient samples and other patients treated with the implicated drug product (DP) lot, (2) in the clinical DP lots, and (3) in their corresponding PBMCs prior to TALEN editing. The primers were optimized using a synthetic template derived from the NGS sequence information, as further described herein. The primers and probe for centromeric BP1 are shown in Table 1D below. The expected PCR product size is 131 bp.

TABLE 1D

| Centromeric | | |
|---|---|---|
| inversion | | SEQ ID |
| Breakpoint 1 | DNA sequence | NO |
| Forward Primer | AGCAGCCAAATCCTTCAGTC | 4 |
| Reverse Primer | GCAGCAAGGTTTTTGTCTGG | 5 |
| Probe | ACTCACAGCTGGGGGATGCC* | 6 |

*5′ FAM, 3′ ZEN/Iowa Black FQ Quencher

As part of the initial method development, primers and probes for the ddPCR assay were designed and optimized. The synthetic template was titrated into human genomic DNA without the inversion and analyzed for specificity and sensitivity to the inversion sequence. The ddPCR assay was found to detect the signal from the synthetic template down to a sensitivity level of approximately 0.1% (Table 1B above; Dong et al. 2018). Increasing amounts of input DNA from drug product and associated peripheral blood mononuclear cells (PBMCs) were also tested for the inversion (Table 1E).

TABLE 1E
Telomeric inversion ddPCR analysis of Drug Product Lot and associated PBMC
material

| | | Expected | | | | Telomeric | | | % Telomeric | | | |
|---|---|---|---|---|---|---|---|---|---|---|---|---|
| | Amount | copies | | | | breakpoint | | | breakpoint relative | | | |
| | of | of | RPP30 Copies/Reaction | | | (Copies/Reaction) | | | to RPP30 | | | Data |
| Sample | gDNA | RPP30 | R1 | R2 | R3 | R1 | R2 | R3 | R1 | R2 | R3 | reporting |
| PBMC | 25 ng | 7600 | 8422.1 | 5768.1 | 5286.0 | 0.0 | 0.0 | 0.0 | 0.0 | 0.0 | 0.0 | ND |
| associated | 50 ng | 15200 | 13836.6 | 12652.9 | 12522.8 | 0.0 | 0.0 | 0.0 | 0.0 | 0.0 | 0.0 | ND |
| with | 100 ng | 30400 | 28817.9 | 24749.5 | 24668.8 | 0.0 | 0.0 | 0.0 | 0.0 | 0.0 | 0.0 | ND |
| Drug | 200 ng | 60800 | 54257.8 | 51094.5 | 48793.2 | 0.0 | 0.0 | 17.7 | 0.0 | 0.0 | 0.04 | ND |
| product | | | | | | | | | | | | |
| Drug | 25 ng | 7600 | 5826.2 | 5357.5 | 5498.1 | 0.0 | 0.0 | 3.97 | 0.0 | 0.0 | 0.07 | ND |
| product | 50 ng | 15200 | 11771.3 | 11403.3 | 11022.2 | 0.00 | 0.00 | 0.00 | 0.00 | 0.00 | 0.00 | ND |
| | 100 ng | 30400 | 22113.9 | 22014.9 | 22188.4 | 0.00 | 0.00 | 0.00 | 0.00 | 0.00 | 0.00 | ND |
| | 200 ng | 60800 | 49256.0 | 45389.2 | 46498.2 | 3.93 | 0.00 | 0.00 | 0.00 | 0.00 | 0.00 | ND |

ND = Not detected,
% Inversion < LOD (0.1%),
R1, R2, and R3 refers to three replicates per sample

Based on this data, 50 ng of human genomic DNA corresponding to ˜ 15,000 human genome copies were used as the input per reaction. Some patient samples yielded lower quality and/or concentration of DNA, and the input DNA amount was adjusted accordingly. Amplified copies of inversion junctions and RPP30 were measured by a ddPCR droplet reader. Each ddPCR plate consisted of a negative control (NEG CTRL: TaqMan™ Control Genomic DNA), positive control (POS CTRL: Synthetic template was spiked into the TaqMan™ Control Genomic DNA), and a no template control (NTC: water). The frequency of inversion was measured as percentage of the total genome copies of the reference gene (RPP30). ddPCR was performed using a Bio-Rad QX200 Droplet Digital PCR system (Bio-Rad Laboratories, Hercules, CA) according to manufacturer's instructions.

Inversion percentage=(BP copy number/RPP30 copy number)*100

### Example 2—Assay Development for Detecting and Quantifying Chromosomal Translocation

Because ALLO-501A was gene edited at both the TRAC locus (chromosome 14) and the CD52 locus (chromosome 1), chromosomal translocation t (1; 14) (p36; q11.2) may arise from cleavage at the known on-target TALEN CD52 and TRAC binding sites.

A sensitive ddPCR method was developed to quantify this expected though low incidence translocation that may occur as a result of TALEN mediated on-target gene editing of CD52 on chromosome 1 at p36 and TRAC on chromosome 14 at q11.2. When end-to-end joining occurs between edited sites, it can result in 2 predominant and balanced chromosome translocations termed Tl and T4 (see FIG. 5). T4 results from the translocation at the break in the CD52 gene edited site from chromosome 1 to chromosome 14, retaining the chromosome 1 centromere, and T1 is the reciprocal translocation of a break in the TRAC gene edited site retaining the chromosome 14 centromere. This balanced, reciprocal translocation was detected as a clonal chromosomal variant by G-banding karyotyping. The translocation was also detected as a chromosomal rearrangement of the TCRA/D TRAC locus (14q11.2) by FISH. Each GMP lot has also been tested in the development ddPCR assay. GMP drug product (DP) batches and matched starting material PBMCs were tested with the pre-qualified development assays. An RPP30 gene primer-probe set capable of detecting the endogenous human RPP30 gene sequence is also included in every T4 and T1 t (1; 14) (p36; q11.2) translocation ddPCR test sample as an internal reference to express translocation frequency as a percentage relative to the RPP30 gene copy number. The LOQ of each assay was established with a CD52 and TRAC TALEN edited control sample and determined to be 0.20% for T1 and 0.21% for T4 (or 2 in 1000 copies of RPP30).

In addition, each t (1; 14) (p36; q11.2) translocation ddPCR analysis also includes the following test controls to ensure proper performance and reliability of reported results:
Positive Control (PC): ALLO-501A development lot cells collected on day 8 (2 days after electroporation of the TALEN mRNA).
Negative Control (NC): ALLO-501A development lot cells collected on day 6 (before electroporation of the TALEN mRNA)
Non-template control

The primers and probe for T1 and T4 are shown below.

T1 Forward primer:
(SEQ ID NO: 7)
AGGCCTGGCCGTGAA
T1 Reverse primer:
(SEQ ID NO: 8)
CCCAGCCAGGGCCTTA
T1 Probe:
(SEQ ID NO: 9)
ACTTGCCAGCCCCACAGAGCCCC
5′ FAM, 3′ ZEN/Iowa Black FQ Quencher.
T4 Forward primer:
(SEQ ID NO: 10)
AGCAGTGCAGCCCCA
T4 Reverse primer:
(SEQ ID NO: 11)
CTCCAGGCCACAGCAC
T4 Probe:
(SEQ ID NO: 12)
CCAGGCCCGCTGTGTCCCCAGTT
5′ FAM, 3′ ZEN/Iowa Black FQ Quencher.

Genomic DNA was extracted from cells using a DNA extraction kit as described in Example 1. The results show that a low level of the expected reciprocal T4 and T1 translocations can be detected in each GMP lot.

In summary, the expected translocation t (1; 14) (p36; q11.2) is a result of CD52 and TRAC TALEN on-target gene editing. The implementation of the T4 and T1 t(1; 14)(p36; q11.2) translocation ddPCR assays may provide a more sensitive and precise estimate of this event than a G-banding karyotype assay and thus provides an alternative assessment of lot-to-lot consistency of the TALEN gene-editing process-step. Sensitive development ddPCR assays for T4 and T1 (pre-qualification LOQ of 0.21 and 0.20%, respectively) have been established.

### Example 3—Methods for Detecting Additional Properties of Engineered Cells

An IL-2 independent proliferation assay was designed to detect the aberrant proliferation of CAR T cells (sensitivity of 1 aberrant cell in 100,000 cells) that may be present in the drug product (DP) as a result of genetic manipulation, cell activation and/or propagation during the manufacturing process was used to evaluate test samples. Without target activation and the addition of IL-2, the CAR T cells do not proliferate (Table 3A).

A positive control was included in the assay to control cell culture conditions for the test samples and to establish validity of the test run. In this assay, 2×10⁶ ALLO-501A test sample cells were cultured in a flask in the absence of target cells and IL-2 for at least 18 days (up to 28 days). The number of viable cells at day 0, at day 18 or at day 28 were counted using the NC200 NucleoCounter, a compact instrument with dual fluorescence channels designed to detect signals from total and dead cells stained with Acridine orange and Diamidino-2-Phenylindole (DAPI), respectively. The total number of viable cells at different timepoints were compared to day 0, and the fold expansion was calculated. A fold expansion is reported as a number if the cell count is within the NC200 NucleoCounter approved counting assay range for CAR T cells (i.e., 1×105-2×106 cells/mL). When the cell count on day 18 is below the approved cell count assay range, the fold expansion is <0.5. The result is reported as “No aberrant proliferation detected at day 18” and meets product specification, when the proliferation fold is less than 0.5. The result is reported as “Aberrant proliferation detected at day 18” and fails the product specification if the proliferation fold is equal to or higher than 0.5. ALLO-501A CAR T cells were also cultured in the presence of CD19-positive target cells (Irradiated Daudi cells), and in the presence of IL-2 at a concentration of 20 ng/mL as a test sample positive control. This arm of the assay confirms that the DP, when stimulated by target cells and IL-2, expands as expected. Cell proliferation was monitored for at least 18 days (or up to 28 days). The total number of viable cells at different timepoints were compared to day 0, and the fold expansion was calculated. A test sample cultured in the presence of target cells and IL-2 must have a fold expansion>2.0-fold as a test sample suitability criterion.

System suitability of each assay run is evaluated by monitoring the proliferation of an approved control CAR T cells sample. Specifically, the positive control CAR T sample must have a fold expansion<0.5-fold in absence of target cells and IL-2 but a fold expansion>2.0-fold in the presence of target cells and IL-2.

Critical reagents utilized in this method including Fetal Bovine Serum (FBS), Irradiated Daudi Cells (target cells), ALLO-501A positive control batch, and IL-2 are all qualified for usage per critical reagents internal procedures. The method has been qualified as a limit test. A detection limit of 20 aberrant cells (CCRF-CEM cell line, Human T lymphoblast cells [ATCC CCL-119], referred to as CEM cells) in 2×10⁶ test sample cells has been evaluated and confirmed during qualification. The method was demonstrated to meet repeatability precision and intermediate precision acceptance criteria for the non-spiked test samples (proliferation fold<0.5) as well as for the test samples spiked with 20 aberrant cells (proliferation fold≥0.5). Specificity was confirmed from the non-spiked and spiked test samples with aberrant cells, demonstrating that proliferation is observed only in the test sample that is spiked. Based on the method qualification demonstrating a detection limit of 20 aberrant cells in 20×10⁶ test sample cells, the CEM spike control is not routinely included in the execution and the method confirmed to be suitable for its intended use.

An evaluation of test samples of ALLO-501A GMP lots (Lots 1 and 2) that were infused into the patient was performed over a longer duration (e.g., 28 days) of proliferation in the absence of IL-2. As a control, an 18-day arm was performed in accordance with the qualified release method. In addition, a spike of 20 aberrant cells (CEM cells) was included in the 18-day duration arm for each lot to serve as a positive control with a sensitivity of detection of 1 in 100,000 cells. For the 28-day assay, 5 and 10 CEM cells were spiked in for respective sensitivities of 1 in 400,000 and 1 in 200,000 cells. All samples, including test sample in the presence of irradiated Daudi target cells (TC) and IL-2, and test sample spiked with CEM cells, from each arm of the study and for each ALLO-501A lot in scope were collected on days 18 and 28 and the cells counted. A fold proliferation value was calculated based on the cell number on the respective end of the runs in comparison to the day 0 cell count.

As shown in Table 3A, the test sample in the presence of 20 CEM cells demonstrated detectable CEM proliferation on day 18 for both lots and in both runs, consistent with the qualification of the method, but no aberrant proliferation was detected in the test sample-only arm of the test, confirming the absence of detectable (less than 1 in 100,000) aberrantly-growing cells in the test sample from either DP lot.

For the day 28 analysis, both CEM spike conditions of 5 and 10 cells showed detectable proliferation, confirming the sensitivity of the assay as 1 in 400,000 and 1 in 200,000, respectively. Importantly, no aberrant cell growth in lots 1 and 2 was detected even under this extended culture duration. In all cases, the test samples, when stimulated with irradiated Daudi cells and IL-2, were able to proliferate as expected based on the established mechanism of action.

The research inversion ddPCR assay was used to test for the presence of the chromosome 14 inversion since it has been demonstrated to be sensitive, specific and requires smaller amounts of DNA than sequencing (which was not practical on these low cell density and viability samples). DNA isolated from samples from both DP lots at day 18 and 28, were tested in this assay and, importantly, chromosome 14 inversion (Table 3A) was undetectable even under the 28-day extended culture duration.

TABLE 3A
Fold Proliferation and ddPCR Chromosome 14 Inversion Results

| | | | ddPCR | | ddPCR |
|---|---|---|---|---|---|
| | | | % | | % |
| | | | Inversion | | Inversion |
| | | Fold | Relative | Fold | Relative |
| Run/Lot | | proliferation | to RPP30 | proliferation | to RPP30 |
| Number | Sample | at Day 18 | at Day 18 | at Day 28 | at Day 28 |
| Run 1/ | TS | <0.5 | Not | <0.5 | Not |
| Lot 1 | | | detected | | detected |
| | TS + TC | 118.1 | | 162.8 | |
| | and IL-2 | | | | |
| | TS + | 1.6 | | 116.1 | |
| | CEM (20 | | | | |
| | in 18-day | | | | |
| | run or 5 | | | | |
| | in 28-day | | | | |
| | run) | | | | |
| Run 2/ | TS | <0.5 | Not | <0.5 | Not |
| Lot 1 | | | detected | | detected |
| | TS + TC | 184.4 | | 289.2 | |
| | and IL-2 | | | | |
| | TS + | 0.8 | | 166.7 | |
| | CEM (20 | | | | |
| | in 18-day | | | | |
| | run or 10 | | | | |
| | in 28-day | | | | |
| | run) | | | | |
| Run 1/ | TS | <0.5 | Not | <0.5 | Not |
| Lot 2 | | | detected | | detected |
| | TS + TC | 127.9 | | 112.8 | |
| | and IL-2 | | | | |
| | TS + | 0.7 | | 41.8 | |
| | CEM (20 | | | | |
| | in 18-day | | | | |
| | run or 5 | | | | |
| | in 28-day | | | | |
| | run) | | | | |
| Run 2/ | TS | <0.5 | Not | <0.5 | Not |
| Lot 2 | | | detected | | detected |
| | TS + TC | 100.6 | | 66.6 | |
| | and IL-2 | | | | |
| | TS + | 1.9 | | 103.1 | |
| | CEM (20 | | | | |
| | in 18-day | | | | |
| | run or 10 | | | | |
| | in 28-day | | | | |
| | run) | | | | |

TS = test sample; TC = target cells (irradiated Daudi target cells)

1. A method for detecting a genome abnormality in an engineered cell population comprising:
a) providing genomic deoxyribonucleic acid (gDNA) molecules from an engineered cell population, wherein the engineered cell population (i) is suspected of comprising a chromosome 14 inversion characterized by a centromeric inversion site at the T cell receptor alpha/delta locus (TCR A/D) and a telomeric inversion site at the immunoglobulin heavy chain (IGH) variable region and (ii) comprises an edited genomic region;
b) subjecting a gDNA molecule to conditions sufficient to generate a nucleic acid extension product, wherein the nucleic acid extension product comprises a sequence corresponding to the chromosome 14 inversion or a complement thereof; and
c) detecting the nucleic acid extension product as an indicator of the presence of the chromosome 14 inversion in the engineered cell population.

2. The method of claim 1, wherein the centromeric inversion site is present at a first genomic region.

3. The method of claim 2, wherein the telomeric inversion site is present at a second genomic region.

4. The method of claim 3, wherein the edited genomic region is present at a third genomic region.

5. The method of claim 4, wherein the third genomic region is different from the first genomic region or the second genomic region.

6. The method of claim 4, wherein the third genomic region is different from the first genomic region and the second genomic region.

7. The method of claim 1, wherein the centromeric inversion site is at the T cell receptor joining gene TRAJ7.

8. The method of claim 1, wherein the TCR A/D locus is at 14q11.2.

9. The method of claim 1, wherein the telomeric inversion site is at the IGHV3-69-1 pseudogene.

10. The method of claim 1, wherein the IGH variable region is at 14q32.

11. The method of claim 1, wherein the chromosome 14 inversion is located between TRAJ7 and IGHV3-69-1.

12. The method of claim 1, wherein the chromosome 14 inversion is a 14q11 inversion.

13. The method of claim 1, wherein the chromosome 14 inversion occurred independent of a gene-editing process.

14. The method of claim 13, wherein the gene-editing process is an in vitro or an in vivo gene-editing process.

15. (canceled)

16. The method of claim 1, further comprising subjecting the gDNA to conditions sufficient to generate a control nucleic acid extension product.

17. The method of claim 16, further comprising detecting the control nucleic acid extension product as an indicator that the conditions were sufficient to generate the control nucleic acid extension product.

18. (canceled)

19. (canceled)

20. (canceled)

21. (canceled)

22. The method of claim 16, wherein the control nucleic acid extension product comprises a sequence corresponding to an endogenous gene sequence.

23. The method of claim 22, wherein the endogenous gene sequence is a human ribonuclease P protein subunit p30 (RPP30) gene sequence.

24. The method of claim 6, wherein the first genomic region and/or the second genomic region are separated from the third genomic region by at least about 5 kilobases (kb), about 6 kb, about 7 kb, about 8 kb, about 9 kb, about 10 kb, about 11 kb, about 12 kb, about 13 kb, about 14 kb, or about 15 kb.

25. The method of claim 1, further comprising extracting gDNA from the population of cells prior to step a).

26. The method of claim 25, further comprising fragmenting the gDNA to provide the gDNA molecules.

27. The method of claim 1, wherein the engineered cell population is an engineered immune cell population.

28. The method of claim 27, wherein the engineered immune cell population comprises a chimeric antigen receptor (CAR) nucleic acid sequence.

29. The method of claim 28, wherein the CAR nucleic acid sequence expresses a CAR that binds to BCMA, EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, FLT3, CD70, DLL3, CD52 or CD34.

30. A method for detecting a genome abnormality in an engineered cell population comprising:
a) providing gDNA molecules from an engineered cell population, wherein the engineered cell population (i) is suspected of comprising a chromosome 14 inversion characterized by a centromeric inversion site at the T cell receptor alpha/delta locus (TCR A/D) and a telomeric inversion site at the immunoglobulin heavy chain (IGH) variable region and (ii) comprises an edited genomic region;
b) generating a plurality of partitions, wherein a partition comprises a gDNA molecule and primer molecules that are complementary to a genomic region corresponding to the chromosome 14 inversion;
c) in the partition, generating amplicons from the gDNA molecule with the primer molecules, wherein the amplicons comprise sequences corresponding to the chromosome 14 inversion; and
d) detecting the amplicons in the partition.

31-59. (canceled)

60. An in vitro method of detecting a genome abnormality in a patient cell population comprising:
a) providing genomic deoxyribonucleic acid (gDNA) molecules from a patient cell population, wherein the patient cell population comprises an immune cell which (i) is suspected of comprising a chromosome 14 inversion characterized by a centromeric inversion site at the T cell receptor alpha/delta locus (TCR A/D) and a telomeric inversion site at the immunoglobulin heavy chain (IGH) variable region and (ii) comprises an edited genomic region;
b) subjecting a gDNA molecule to conditions sufficient to generate a nucleic acid extension product, wherein the nucleic acid extension product comprises a sequence corresponding to the chromosome 14 inversion or a complement thereof; and
c) detecting the nucleic acid extension product as an indicator of the presence of the chromosome 14 inversion in the immune cell.

61-79. (canceled)

80. A drug product release assay for an allogeneic immune cell drug product, wherein the release assay comprises:
a) providing an allogeneic immune cell drug product comprising engineered immune cells that comprise edited genomic regions;
b) extracting gDNA molecules from the engineered immune cells;
c) subjecting a gDNA molecule to conditions sufficient to generate a nucleic acid extension product that comprises a sequence corresponding to a chromosome 14 inversion characterized by a centromeric inversion site at the T cell receptor alpha/delta locus (TCR A/D) and a telomeric inversion site at the immunoglobulin heavy chain (IGH) variable region, or a complement thereof; and
d) detecting the nucleic acid extension product as an indicator of the presence of the chromosome 14 inversion in the engineered immune cells.

81-104. (canceled)
